(19) United States
(12) Patent Application Publication
(10) Pub. No.: US 2022/0288235 A1
(43) Pub. Date: Sep. 15, 2022

(54) SCHIZOPHRENIA-RELATED MICRODELETION GENE 2510002D24Rik IS ESSENTIAL FOR SOCIAL MEMORY

(71) Applicant: St. Jude Children's Research Hospital, Inc., Memphis, TN (US)

(72) Inventors: Stanislav S. ZAKHARENKO, Collierville, TN (US); Prakash DEVARAJU, Memphis, TN (US)

(73) Assignee: St. Jude Children's Research Hospital, Inc., Memphis, TN (US)

(21) Appl. No.: 17/636,152

(22) PCT Filed: Aug. 19, 2020
(86) PCT No.: PCT/US20/46976

§ 371 Date: Feb. 17, 2022

Related U.S. Application Data
(60) Provisional application No. 62/889,111, filed on Aug. 20, 2019.

(51) Int. Cl. A61K 48/00; C07K 14/47; A61P 25/18

(52) U.S. Cl. CPC A61K 48/0058; C07K 14/47; A61P 25/18; C07K 2319/42; C12N 2750/14143

(57) ABSTRACT

Described is a method for treating a social memory deficit in a subject with a neuropsychiatric disease is treated by administering a peptide encoded by 2510002D24Rik or Atp23 genes, a vector expressing such peptide, or an agent capable of increasing the level or activity of such peptide. The method may be used to treat schizophrenia or autism. The peptide, vector or agent can be administered directly to the hippocampus, such as by transcranial surgical injection.

### CROSS-REFERENCE TO RELATED APPLICATIONS

This application claims priority to U.S. Provisional Application No. 62/889,111, filed Aug. 20, 2019, the disclosure of which is herein incorporated by reference in its entirety.

### STATEMENT AS TO FEDERALLY SPONSORED RESEARCH

This invention was made with government support under MH097742 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jul. 6, 2020, is named 243734_000139_SL.txt and is 17,315 bytes in size.

### FIELD

This application relates to compositions for use in treating schizophrenia spectrum disorders, autism spectrum disorder, and any other disorder or condition arising from a social memory deficit. The application further relates to methods of treating schizophrenia spectrum disorders, autism spectrum disorder, and any other disorder or condition arising from a social memory deficit.

### BACKGROUND

The 22q11.2 deletion syndrome (22q11DS) is associated with high risk of developing schizophrenia symptoms, including psychosis, later in life. 22q11DS is a leading genetic cause of schizophrenia and instigated by the hemizygous deletion of multiple genes (1.5-3 Mb) of the q (long) arm of chromosome 22 in humans. In particular, 22q11DS carries a 25- to 30-fold risk of schizophrenia.⁹⁻¹¹

Schizophrenia may cause specific changes in area CA2, a long over-looked region of the hippocampus recently found to be critical for social memory formation. Silencing or lesioning the CA2 area of the murine hippocampus is known to cause a specific deficit in social-recognition memory, with no change in sociability or spatial and contextual memories typically associated with the hippocampus.^{1,5}

### SUMMARY OF THE INVENTION

There is a great need in the art to develop effective treatments for 22q11DS, including treatment of positive symptoms of mental disorders and schizophrenia, such as, e.g., hallucinations, delusions, disorganized thought, and psychosis. The present invention addresses this and other needs.

In one aspect is provided a method for treating a social memory deficit in a subject with a neuropsychiatric disease, the method comprising administering to the subject a therapeutically effective amount of (i) a protein encoded by a 2510002D24Rik gene or a functional derivative or fragment thereof, or (ii) a vector expressing said protein encoded by said 2510002D24Rik gene or a functional derivative or fragment thereof.

In another aspect is provided a method for treating a social memory deficit in a subject with a neuropsychiatric disease, said method comprising administering to the subject a therapeutically effective amount of (i) a protein encoded by an Atp23 gene or a functional derivative or fragment thereof, or (ii) a vector expressing said protein encoded by said Atp23 gene or a functional derivative or fragment thereof.

In some embodiments of the above aspects, the administration results in replenishing Atp23 level in CA2 interneurons of the subject. In a specific embodiment, the CA2 interneurons are parvalbumin (PV)-positive interneurons.

In various embodiments, the administration results in replenishing Atp23 level in CA2 area of the hippocampus of the subject. In some embodiments, replenishing the Atp23 level is to the level found in healthy subjects.

In some embodiments, the neuropsychiatric disease is selected from schizophrenia spectrum disorders, 22q11 deletion syndrome, and autism spectrum disorders. In some embodiments, the vector is selected from adeno-associated virus (AAV) vectors, retrovirus vectors, adenovirus vectors, Sindbis virus vectors, vaccinia virus vectors, and herpes virus vectors. In a specific embodiment, the vector is an AAV vector. In some embodiments, the AAV vector has a capsid from a serotype selected from AAV1, AAV2, AAV5, AAV8, and AAV9. In certain embodiments, the retrovirus vector is a lentivirus vector.

In various embodiments, the expression of the protein or functional derivative or fragment thereof in the vector is controlled by a promoter selected from the group consisting of fsst promoter, hDlx promoter, mDlx promoter, Synapsin promoter, CMV promoter, β-actin promoter, and CamKIIa promoter. In some embodiments, the expression of the protein or functional derivative or fragment thereof in the vector is controlled by a pan-GABAergic interneuron promoter.

In some embodiments, the administration is via injection into the CA2 area of the hippocampus of the subject. In some embodiments, the administration is via a transcranial surgical injection. In specific embodiments, the administration is systemic. In specific embodiments, the administration is intranasal.

In some embodiments, the protein encoded by 2510002D24Rik gene comprises the amino acid sequence which has at least 80% sequence identity to SEQ ID NO: 1. In some embodiments, the protein encoded by 2510002D24Rik gene comprises the amino acid sequence which has at least 90% sequence identity to SEQ ID NO: 1. In some embodiments, the protein encoded by 2510002D24Rik gene comprises the amino acid sequence SEQ ID NO: 1. In some embodiments, the protein encoded by 2510002D24Rik gene consists of the amino acid sequence SEQ ID NO: 1.

In some embodiments, the protein encoded by Atp23 gene comprises the amino acid sequence which has at least 80% sequence identity to SEQ ID NO: 2. In some embodiments, the protein encoded by Atp23 gene comprises the amino acid sequence which has at least 90% sequence identity to SEQ ID NO: 2. In some embodiments, the protein encoded by Atp23 gene comprises the amino acid sequence SEQ ID NO: 2. In some embodiments, the protein encoded by Atp23 gene consists of the amino acid sequence SEQ ID NO: 2.

In various embodiments, the subject is human.

In another aspect is provided a pharmaceutical composition comprising a protein encoded by 2510002D24Rik gene or a functional derivative or fragment thereof and a pharmaceutically acceptable carrier or excipient.

In another aspect is provided a pharmaceutical composition comprising a vector encoding a protein encoded by 2510002D24Rik gene or a functional derivative or fragment thereof and a pharmaceutically acceptable carrier or excipient. In some embodiments, the vector is selected from adeno-associated virus (AAV) vectors, retrovirus vectors, adenovirus vectors, Sindbis virus vectors, vaccinia virus vectors, and herpes virus vectors. In certain embodiments, the vector is an AAV vector. In some embodiments, the AAV vector has a capsid from a serotype selected from AAV1, AAV2, AAV5, AAV8, and AAV9. In certain embodiments, the retrovirus vector is a lentivirus vector.

In some embodiments, in the vector the sequence encoding the protein encoded by 2510002D24Rik gene or functional derivative or fragment thereof is operably linked to a promoter selected from the group consisting of fsst promoter, hDlx promoter, mDlx promoter, Synapsin promoter, CMV promoter, β-actin promoter, and CamKIIa promoter. In some embodiments, in the vector the sequence encoding the protein encoded by 2510002D24Rik gene or functional derivative or fragment thereof is operably linked to a pan-GABAergic interneuron promoter. In some embodiments, the protein encoded by 2510002D24Rik gene comprises the amino acid sequence which has at least 90% sequence identity to SEQ ID NO: 1. In some embodiments, the protein encoded by 2510002D24Rik gene comprises the amino acid sequence SEQ ID NO: 1. In some embodiments, the protein encoded by 2510002D24Rik gene consists of the amino acid sequence SEQ ID NO: 1.

In another aspect is provided a pharmaceutical composition comprising a protein encoded by Atp23 gene or a functional derivative or fragment thereof and a pharmaceutically acceptable carrier or excipient.

In another aspect is provided a pharmaceutical composition comprising a vector encoding a protein encoded by Atp23 gene or a functional derivative or fragment thereof and a pharmaceutically acceptable carrier or excipient. In some embodiments, the vector is selected from adeno-associated virus (AAV) vectors, retrovirus vectors, adenovirus vectors, Sindbis virus vectors, vaccinia virus vectors, and herpes virus vectors. In certain embodiments, the vector is an AAV vector. In some embodiments, the AAV vector has a capsid from a serotype selected from AAV1, AAV2, AAV5, AAV8, and AAV9. In certain embodiments, the retrovirus vector is a lentivirus vector.

In various embodiments, in the vector the sequence encoding the protein encoded by Atp23 gene or functional derivative or fragment thereof is operably linked to a promoter selected from the group consisting of fsst promoter, hDlx promoter, mDlx promoter, Synapsin promoter, CMV promoter, β-actin promoter, and CamKIIa promoter. In some embodiments, in the vector the sequence encoding the protein encoded by Atp23 gene or functional derivative or fragment thereof is operably linked to a pan-GABAergic interneuron promoter. In some embodiments, the protein encoded by Atp23 gene comprises the amino acid sequence which has at least 90% sequence identity to SEQ ID NO: 2.

In some embodiments, the protein encoded by Atp23 gene comprises the amino acid sequence SEQ ID NO: 2. In some embodiments, the protein encoded by Atp23 gene consists of the amino acid sequence SEQ ID NO: 2.

In various embodiments, the pharmaceutical composition is formulated for injection into hippocampus. In various embodiments, the pharmaceutical composition is formulated for transcranial surgical injection.

### BRIEF DESCRIPTION OF THE DRAWINGS

FIGS. 1A-1H show the results of experiments on deficits in social memory, CA2 fast-spiking interneuron firing, long-term depression at CA3-CA2 inhibitory synapses, and disinhibitory plasticity in CA2 pyramidal neurons of 2510002D24Rik-deficient mice. In FIGS. 1A and 1B, the left panel shows direct interaction one-chamber test using the same (FIG. 1A) or a different (FIG. 1B) stimulus animal in the 2 trials. In FIG. 1A, the right panel shows that only WT mice displayed decreased investigation during trial 2 of the mouse encountered in trial 1 (WT, n=13, *P=002; Rik^{+/-}, n=19, P=0.331; Rik^{-/-}, n=17, P=0.113). Two-way repeated measures (RM) analysis of variance (ANOVA): F(1,46)=12.133, *P=0.001; trial 1: all pair-wise comparisons P>0.05; trial 2: WT vs. Rik^{+/-}, *P=0.032, WT vs. Rik^{-/-}, *P=0.026. In FIG. 1B, mutant and WT mice explored the 2 different stimulus animals similarly (two-way RM ANOVA: F(1,46)=2.893, P=0.096). FIGS. 1C and 1D show input-output curves of the compound EPSP/IPSP (PSP) amplitudes in control conditions (FIG. 1C) and after blocking inhibition with SR95531 (1 μm) and CGP55845 (1 μm) (FIG. 1D) in response to Schaffer collateral stimulation in WT (c: n=16 neurons in 6 mice; d: 12 neurons, 6 mice), Rik^{+/-} (c: 16 neurons, 6 mice; d: 9 neurons, 4 mice), and Rik^{-/-} (c: 20 neurons, 5 mice; d: 13 neurons, 6 mice) mice. In FIG. 1C, the parameters for RM ANOVA are: F(2;49)=5.998, genotype *P=0.005. In FIG. 1D, the parameters for two-way RM ANOVA are: F(2;31)=0.563, genotype P=0.574). Insets show representative compound PSP traces. FIG. 1E shows mean IPSCs recorded from CA2 pyramidal neurons in response to stimulation of Schaffer collaterals in WT (13 neurons, 3 mice), Rik^{+/-} (9 neurons, 3 mice), and Rik^{-/-} (10 neurons; 2 mice) animals. In FIG. 1E, the parameters for two-way RM ANOVA are: F(2,29)=6.755, genotype *P=0.004. In FIG. 1F, examples (left) and mean numbers (right) of APs fired in response to 150-pA (1 s) depolarization of CA2 interneurons of WT (10 neurons, 4 mice), Rik^{+/-} (12 neurons, 4 mice), and Rik^{-/-} (10 neurons; 4 mice) animals. The parameters for Kruskal-Wallis one-way ANOVA on ranks are: H=9.643; genotype *P=0.008 (WT vs Rik^{+/-} and WT vs Rik^{-/-}: *P<0.05). FIG. 1G shows mean normalized IPSCs recorded in CA2 pyramidal neurons in the voltage-clamp configuration, before and after tetanic stimulation of Schaffer collaterals (100 pulses at 100 Hz twice, arrows) in WT (8 neurons, 5 mice), Rik^{+/-} (12 neurons, 7 mice), and Rik^{-/-} (15 neurons; 7 mice) animals. The parameters for one-way ANOVA are: F(2,32)=6.044, genotype P=0.006 (WT vs. Rik^{+/-}, *P=0.006; WT vs. Rik^{-/-}, *P=0.018). FIG. 1H shows mean normalized PSP recorded in CA2 pyramidal neurons in the current-clamp configuration, before and after tetanic stimulation of Schaffer collaterals (arrows) in WT (7 neurons, 4 mice), Rik^{+/-} (6 neurons, 3 mice), and Rik^{-/-} (6 neurons; 3 mice) animals. The parameters for one-way ANOVA are: F(2,16)=5.662, genotype P=0.014 (WT vs. Rik^{+/-}, *P=0.026; WT vs. Rik^{-/-}, *P=0.027). In FIGS. 1G and 1H, the insets show representative traces of IPSPs (FIG. 1G) and PSPs (FIG. 1H) before (dark traces) and after (light traces) tetanic stimulation.

FIGS. 2A-2H show the results of experiments demonstrating that deficiency of the 2510002D24Rik-encoded product, which interacts with a mitochondrial protein Atp23, reduces ATP-ADP interconversion in CA2 interneurons. FIGS. 2A and 2B show the results of a tandem mass tag (TMT) mass spectrometry-based proteomics analysis in whole-hippocampal tissue (FIG. 2A) and hippocampal synaptosomes (FIG. 2B) from WT and Rik^{-/-} mice revealed 3 hits encoded by 2510002D24Rik and Atp23 genes (red) that are substantially reduced in mutants. In FIG. 2C, Western blotting confirmed the reduction of Atp23 in the whole-hippocampal lysate of Rik^{-/-} mice. FIG. 2D shows that Atp23 co-precipitates with 2510002D24Rik^{HA} fusion protein (HA) in the hippocampal lysates from Rik^{HA} mice. The Atp23 signal was stronger in the HA-pulled immunoprecipitate (IP) than in the input lysate or the flow-through (FT) fraction that remained after immunoprecipitation. FIG. 2E shows enrichment of 2510002D24Rik^{HA} fusion protein (HA) and Atp23 in mitochondrial fractions (M) compared to synaptosomal fractions (S) prepared from the hippocampus of Rik^{HA} mice. FIG. 2F shows examples (top) and averages (bottom) of PercevalHR fluorescence excited at 840 nm (ADP, upward sweeps) or 940 nm (ATP, downward sweeps) before, during, and after 300-pA depolarization measured through line scans in cell bodies of CA1 interneurons of WT and 2510002D24Rik-deficient mice. In FIGS. 2G and 2H, the mean area under curves (AUC) for 840-nm-excited (FIG. 2G) and 940-nm-excited (FIG. 2H) PercevalHR fluorescence in WT (n=7); Rik^{/-} (n=7), and Rik^{-/-} (n=7) neurons. In FIG. 2G, the parameters for one-way ANOVA are: F(2;18)=4.251, genotype *P=0.031 (WT vs. Rik^{+/-}, *P=0.033, WT vs. Rik^{-/-}, *P=0.027). In FIG. 2H, the parameters for one-way ANOVA are: one-way ANOVA: F(2,18)=7.9, genotype *P=0.003 (WT vs. Rik^{-/-}, *P=0.003, WT vs. Rik^{-/-}, *P=0.007).

FIGS. 3A-3H show that Atp23 deletion or 2510002D24Rik conditional deletion causes deficits in firing and synaptic plasticity in CA2 interneurons and social memory. FIG. 3A shows examples of firing (left) and mean numbers of APs (right) induced by 150-pA (1 s) depolarization in CA2 interneurons of WT (14 neurons, 3 mice) and Atp23^{+/-} (17 neurons, 3 mice) animals. Mann-Whitney Rank-Sum test: U=56.5, *P=0.014. FIG. 3B shows average normalized IPSCs measured before and after tetanic stimulation (arrows) of Schaffer collaterals in WT (12 neurons, 7 mice) and Atp23^{-/-} (7 neurons, 4 mice) CA2 pyramidal neurons. Two-tailed t-test t=−2.428, *P=0.028. FIGS. 3C and 3D show the results of a direct interaction one-chamber test using the same (FIG. 3C) or different (FIG. 3D) stimulus animal in the 2 trials in WT (n=12) and Atp23^{+/-} (n=11) mice. In FIG. 3C, there was a significant difference between the groups. Two-way RM ANOVA: trial F(1,21)=11.621, *P=0.003. WT (*P<0.001) but not Atp23^{+/-} (*P=0.374) mice showed less investigation during trial 2 than in trial 1. There was no difference between genotypes in trials 1 (P=0.062) or 2 (P=0.816). FIG. 3D shows the results of an experiment in which WT and Atp23^{+/-} mice explored the 2 different stimulus animals similarly. Two-way RM ANOVA: F(1,21)=2.757, P=0.112. FIG. 3E shows examples of firing (left) and mean numbers of APs (right) induced by 150-pA (1 s) depolarization in labelled CA2 interneurons of WT (11 neurons, 3 mice) and Rik^{f/f} mice injected with AAV-hDlx-cre-GFP. Rik^{f/f};CA2^{AVV-hDlx-cre} (n=10 neurons, 3 mice) neurons fired significantly fewer APs than did WT;CA2^{AVV-hDlx-cre} neurons (n=11 neurons, 3 mice). Mann-Whitney Rank-Sum test U=19.5, *P=0.014. In FIG. 3F, the average normalized IPSCs were measured before and after tetanic stimulation (arrows) of Schaffer collaterals in WT;CA2^{AVV-hDlx-cre} (6 neurons, 3 mice) and Rik^{f/f}; CA2^{AVV-hDlx-cre} (6 neurons, 4 mice) CA2 pyramidal neurons. Two-tailed t-test: t=−2.804, *P=0.019. FIG. 3G shows the results of a direct interaction one-chamber test using the same stimulus animal in the 2 trials revealed that WT;CA2^{AVV-hDlx-cre} (n=5, *P=0.027) mice but not Rik^{f/f}; CA2^{AVV-hDlx-cre} (n=6, P=0.616) mice showed less investigation between the trials. Two-way RM ANOVA: F(1,9)=6.01, *P=0.037. In FIG. 3H, a direct interaction one-chamber test using different stimulus animals in the 2 trials revealed no difference between WT;CA2^{AVV-hDlx-cre} (n=4) and Rik;CA2^{AVV-hDlx-cre} (n=5) mice. Two-way RM ANOVA: F(1,7)=1.342, P=0.361. Insets (FIGS. 38 and 3F) show representative traces of IPSCs before (solid lines) and after (dash lines) tetanic stimulation.

FIGS. 4A-4F show that replenishing Atp23 in CA2 interneurons rescues deficiencies in interneuron firing, CA3-CA2 synaptic plasticity, and social memory in 2510002D24Rik-deficient mice. FIG. 4A is an image of the hippocampus infected with AAV-fsst-Atp23-GFP (green) in the CA2 area, which is marked by RGS14 (red). The scale bar represents 200 μm. FIG. 4B shows examples and mean numbers of APs in labelled CA2 interneurons infected with AAV-fsst-RFP [CA2^{AVV-fsst-RFP} (RFP)] or AAV-fsst-Atp23-GFP [CA2^{AVV-fsst-Atp23} (Atp23-GFP)] in WT (11 neurons, 6 neurons), Rik^{+/-} (9 neurons, 10 neurons), and Rik^{-/-} (8 neurons, 11 neurons) mice. RFP: one-way ANOVA, F(2,24)=4.737, *P=0.018. WT vs. Rik^{+/-} (*P=0.035), WT vs Rik^{-/-} (*P=0.037). Atp23-GFP: one-way ANOVA, F(2,24)=0.215, P=0.808. FIG. 4C shows average normalized IPSCs measured before and after tetanic stimulation (arrows) of Schaffer collaterals in CA2 pyramidal neurons of WT (n=10, n=6), Rik^{+/-} (8 neurons, 6 neurons) Rik^{-/-} mice (11 neurons, 7 neurons) injected with AAV-fsst-RFP (RFP, left) or AAV-fsst-Atp23-GFP (Atp23-GFP, right). RFP: one-way ANOVA, F(2,25)=7.792, *P=0.002. WT vs Rik^{+/-}, *P=0.003, WT vs. Rik^{-/-}, *P=0.037. Atp23-GFP: one-way ANOVA, F(2,16)=2.313, P=0.131. FIG. 4D shows mean normalized PSP recorded in CA2 pyramidal neurons in the current-clamp configuration, before and after delivery of tetanic stimulation of Schaffer collaterals (arrows) in WT (9 neurons, 8 neurons), Rik^{+/-} (9 neurons, 7 neurons), and Rik^{-/-} (7 neurons, 6 neurons) animals injected with AAV-fsst-RFP (RFP, left) or AAV-fsst-Atp23-GFP (Atp23-GFP, right). RFP: Kruskal-Wallis one-way ANOVA on ranks, H=9.373, *P=0.009. WT vs. Rik^{-/-}, *P<0.05; WT vs. Rik^{+/-}, *P<0.05. Atp23-GFP-Kruskal-Wallis one-way ANOVA on ranks, H=0.09, P=0.956. In FIGS. 4C and 4D, insets show representative traces of IPSCs (FIG. 4C) and PSPs (FIG. 4D) before (dark traces) and after (light traces) tetanic stimulation. FIGS. 4E and 4F show direct interaction one-chamber test using the same (FIG. 4E) or a different (FIG. 4F) stimulus animal in the 2 trials.

FIGS. 5A-5F show mice carrying a deletion of the 22q11 deletion syndrome (22q11DS) gene 2510002D24Rik are deficient in the social novelty test but not in sociability. FIG. 5A is a diagram depicting the location of C22orf39 gene in human chromosome 22 and the mouse orthologue 2510002D24Rik in the syntenic region of mouse chromosome 16. The genes deleted in the Df(16)A^{+/-} mouse model are indicated by the solid black line at the bottom. In FIG. 5B the 2510002D24Rik mRNA levels are lower in Rik^{+/-} (n=12) and Rik^{-/-} (n=10) mice than in WT mice (n=10). Kruskal-Wallis one-way ANOVA on ranks: H=29.289, *P<0.001. All pairwise comparisons, *P<0.05. In FIG. 5C, the left panel shows a 3-chamber social novelty test. In FIG. 5C, the right panel shows that WT mice (n=14, *P=0.009) but not Rik^{+/-} (n=30, P=0.99) or Rik^{-/-} (n=19, P=0.855) mice preferred the novel mouse over a familiar mouse. Kruskal-Wallis one-way ANOVA on ranks, H=23.084, *P<0.001. FIG. 5D shows the results of the 3-chamber social preference test. WT (n=11), Rik^{+/-} (n=23), and Rik^{-/-} (n=9) mice explored a novel mouse more compared to an empty chamber. Two-way RM ANOVA: F(1, 41)=18.05, *P<0.001. No difference was detected among genotypes exploring a novel mouse or an empty chamber (Holm-Sidak pairwise comparison method, P>0.05). FIG. 5E shows that mice carrying a deletion of the 22q11DS gene 2510002D24Rik are deficient in the social novelty test but not in sociability. (a) Left: 3-chamber social novelty test. Center: WT mice (n=12, *P=0.002) but not Rik+/− (n=21, P=0.441) or Rik−/− (n=15, P=0.777) mice preferred the novel stimulus mouse over a familiar stimulus mouse. Two-way RM ANOVA: F(1,49)=7.486, *P=0.009). Right: Difference score is reduced in 2510002D24Rik-deficient mice in the 3-chamber social novelty test. One-way ANOVA: F(2, 46)=3.239, *P=0.048. Pair-wise comparisons, WT vs. Rik+/−, *P=0.032, WT vs. Rik−/−, *P=0.049. In the left panel of FIG. 5F, the 3-chamber social preference test. Center: WT (n=12), Rik+/− (n=23), and Rik−/− (n=9). WT and Rik+/− mice similarly explored a novel mouse longer than an empty chamber. Two-way RM ANOVA: F(1,41)=18.049, *P<0.001). In the right panel of FIG. 5F, the difference scores were similar between WT and 2510002D24Rik-deficient mice in the 3-chamber social preference test. One-way ANOVA: F(2, 41)=1.541, P=0.226.

FIGS. 6A-6F shows that the 2510002D24Rik-deficient mice have no deficits in olfactory investigation, motor function, compulsiveness, or anxiety. FIG. 6A shows that there was no difference among WT (n=8), Rik^{+/-} (n=8), and Rik^{-/-} (n=8) mice in performance of the olfactory habituation/dishabituation task. Two-way RM ANOVA: trial F(14,21)=30.392, P<0.001; genotype F(2,21)=0.0368, P=0.964. FIGS. 6B-6E show that WT (n=9) Rik^{+/-} (n=9) and Rik^{-/-} (n=10) mice showed no difference in the open-field exploration task (FIGS. 6B-6C) [time in center vs. time in corners: two-way RM ANOVA, F(2,25)=0.03, P=0.97. In FIGS. 6D and 6E, the number of bouts center vs corners: two-way RM ANOVA, F(2,25)=0.07, P=0.926], in the rotarod task (FIG. 6D) [Left: Kruskal-Wallis one-way ANOVA on ranks, H=2.145, P=0.342. Right one-way ANOVA: F(2,25)=1.281, P=0.295], or grooming time (FIG. 6E) [Left one-way ANOVA, F(2,25)=0.0970, P=0.908. Right: one-way ANOVA, F(2,25)=0.453, P=0.64)]. FIG. 6F shows that no difference was detected in WT (n=9), Rik^{+/-} (n=13), and Rik^{-/-} (n=11) mouse performance in the elevated plus maze task. Two-way RM ANOVA: location F(2,30)=370.5, P<0.001, genotype F(2,30)=0.942, P=0.401.

FIGS. 7A-7G show that the 2510002D24Rik-deficient mice have no deficit in spatial, contextual, or fear memories. FIGS. 7A-7D show that WT (n=20-22), Rik^{+/-} (n=20-22), and Rik^{-/-} (n=21) mice performed equally well in the spatial memory and visual tasks in the Morris water maze. The parameters are as follows. Training (FIG. 7A): two-way RM ANOVA, genotype F(2,62)=0.521, P=0.596. Visible platform task (FIG. 7B): one-way ANOVA, genotype F(2,58)=2.727, P=0.074. Memory one hour probe (FIG. 7C): two-way RM ANOVA, genotype F(2,58)=0.009, P=0.991. Memory 24 hour probe (FIG. 7D): Two-way RM ANOVA, genotype F(2,58)=0.297, P=0.744. FIG. 7E shows that no difference was detected in performance between WT (n=7) and Rik^{-/-} (n=9) mice in the novel object recognition task. Mann-Whitney Rank Sum: U=24, P=0.439. FIGS. 7F and 7G show that WT (n=7), Rik^{+/-} (n=10), and Rik^{-/-} (n=9) mice performed at similar levels in the contextual and cued versions of the fear-conditioning memory tasks. The parameters are as follows. Contextual (FIG. 7F): Kruskal-Wallis one-way ANOVA on ranks, genotype H=0.662, P=0.718. Cued (FIG. 7G): two-way RM ANOVA, treatment F(1, 23)=139.59, P<0.001, genotype F(2,23)=0.237, P=0.791.

FIGS. 8A-8F show normal resting membrane potential and excitability in 2510002D24Rik-deficient mice. Mean resting membrane potential (FIGS. 8A and 8D), input resistance (FIGS. 88 and 8E), and rheobase (FIGS. 8C and 8F) in CA2 pyramidal neurons (FIGS. 8A-8C) and CA2 interneurons (FIGS. 8D-8F). The parameters are as follows. FIG. 8A: Kruskal-Wallis one-way ANOVA on ranks (WT, n=17; Rik^{+/-}, n=16; Rik^{-/-}, n=21), H=2.691, P=0.26. FIG. 8B: One-way ANOVA (WT, n=17; Rik^{+/-}, n=17; Rik^{-/-}, n=21), F(2,52)=1.354, P=0.267. FIG. 8C: Kruskal-Wallis one-way ANOVA on ranks (WT, n=16; Rik^{+/-}, n=17; Rik^{-/-}, n=21), H=4.78, P=0.092. FIG. 8D: One-way ANOVA (WT, n=7; Rik^{+/-}, n=9; Rik^{-/-}, n=8), F(2,21)=0.562, P=0.578. FIG. 8E: One-way ANOVA (WT, n=10; Rik^{+/-}, n=10; Rik^{-/-}, n=12), F(2,29)=0.55, P=0.583. FIG. 8F: Kruskal-Wallis one-way ANOVA on ranks (WT, n=10; Rik^{+/-}, n=10; Rik^{-/-}, n=12), H=0.814, P=0.666.

FIGS. 9A-9B show distal inputs from the entorhinal cortex onto CA2 are not affected in 2510002D24Rik-deficient mice. FIGS. 9A and 9B show averages of input-output curves of the PSP amplitude in control condition (FIG. 9A) and after blocking inhibition with SR95531 and CGP55845 (FIG. 9B) in response to distal stimulation in WT (FIG. 9A: 17 neurons, 6 mice; FIG. 9B: 13 neurons, 6 mice), Rik^{+/-} (FIG. 9A: 16 neurons, 5 mice; FIG. 9B: 9 neurons, 4 mice), and Rik^{-/-} (FIG. 9A: 20 neurons; 5 mice; FIG. 9B: 9 neurons, 4 mice) animals. Two-way RM ANOVA (FIG. 9A): genotype F(2;50)=2.401, P=0.0.101; two-way RM ANOVA (FIG. 9B): F(2;28)=0.212, genotype P=0.811). Insets show representative compound EPSP/IPSP (PSP) traces.

FIGS. 10A-10D show the number of interneurons in the CA2 area of the hippocampus is not altered in 2510002D24Rik-deficient mice. FIG. 10A shows fluorescent images of the hippocampus stained with antibodies against parvalbumin (left), RGS14, a label of the CA2 area (middle), and merged image (right) in WT and Rik^{-/-} mice. FIG. 10B is a high-magnification of parvalbumin staining in the CA2 area (denoted by lines in a and b and identified by RGS14 staining). FIGS. 10C and 10D show mean numbers of PV⁺ interneurons in the CA2 area (FIG. 10C) and the average size of the CA2 area in WT and Rik^{-/-} mice (FIG. 10D). The parameters are as follows. In FIG. 10C, WT, n=4; Rik^{-/-}, n=4, t-test, t=0.182, P=0.862. In FIG. 10D, WT, n=2; Rik^{-/-}, n=2, Mann-Whitney rank-sum test, U=2, P=1.

FIG. 11 shows validation of synaptosomal- and mitochondrial-fractionation procedures. The synaptic marker PSD95 and the mitochondrial marker cytochrome C are expressed in whole (crude) lysate from the hippocampus. Cytochrome C is enriched in the mitochondrial fraction, compared to the synaptosomal fractions (note: synaptosomes also contain mitochondria). PSD95 is enriched in the synaptosomal fractions. Experiments were done in duplicate.

FIGS. 12A, 12B, and 12C show design and validation of the Rik^{HA} knock-in mice. In FIG. 12A, the Rik^{HA} knock-in mouse was produced by inserting the human influenza hemagglutinin (HA) tag upstream of exon 3 of the 2510002D24Rik gene by using the CRISPR/Cas9 approach. FIG. 12B is an immunoblot showing presence of HA in Rik^{HA} mice (n=2) but not in WT animals (n=2). FIG. 12C shows fluorescent images of the sagittal sections of the hippocampus in Rik^{HA} mice stained with anti-HA and anti-RGS14 antibodies. The CA2 area of the hippocampus is denoted by RGS14 staining.

FIGS. 13A-13C show activity-dependent changes in ATP and ADP are not significantly different in CA2 pyramidal neurons. In FIG. 13A, the examples (top) and averages (bottom) of PercevalHR fluorescence excited at 840 nm (ADP) or 940 nm (ATP) before, during, and after 300-pA depolarization measured through line scans in cell bodies of CA1 pyramidal neurons of WT and 2510002D24Rik-deficient mice. FIGS. 13B and 13C show mean area under curves (AUC) of PercevalHR fluorescence. The parameters are as follows. In FIG. 13B, 840 nm-excitation: WT (n=11); Rik^{+/-} (n=8), and Rik^{-/-} (n=11) neurons. Kruskal-Wallis one-way ANOVA on ranks: H=3.809, P=0.149. In FIG. 13C, 940 nm-excitation: WT (n=10); Rik^{+/-} (n=8), and Rik^{-/-} (n=11) neurons. one-way ANOVA: F(2,26)=0.122, P=0.122.

FIG. 14A shows that the 2510002D24Rik protein is localized to mitochondria. Immunostaining for hemagglutinin (HA) and cytochrome C in the CA2 area from Rik^{HA} sagittal hippocampal sections is shown. FIG. 14B shows the number of mitochondria in CA2 inhibitory synapses is unchanged in 2510002D24Rik-deficient mice. Average percent of mitochondrial-containing excitatory (asymmetric) and inhibitory (symmetric) synapses measured in the CA2 area of the hippocampus of WT (665 and 94 synapses, 2 mice) and Rik^{-/-} (547 and 110 synapses, 2 mice) mice. Two-way ANOVA: synapse type, F(1,1)=259.79, P<0.001; genotype, F(1,1)=0.029, P=0.873.

FIGS. 15A and 158 show the design and validation of Atp23 knock-out mice. In FIG. 15A, the Atp23^{+/-} knock-out mouse was produced by deleting exons 2 and 3 from Atp23 by using the CRISPR/Cas9 approach. In FIG. 15B, average normalized levels of Atp23 mRNA was measured by RT-qPCR in the hippocampus of WT (n=8) and Atp23^{+/-} littermates (n=8). t-test: t=10.229, *P<0.001.

### DETAILED DESCRIPTION

The present invention is based on an unexpected discovery that social memory deficit can be ameliorated in mice with 22q11DS (particularly mice deficient in 2510002D24Rik-gene product), by ectopic expression of Atp23 in a CA2 interneuron. Such expression of Atp23 can rescue deficiencies in interneuron firing, CA3-CA2 synaptic plasticity, and social memory in mice deficient in 2510002D24Rik.

### Definitions

As used herein, the term “social memory” means the ability of a mammal, including a human, to recognize a subject encountered previously. Recognition may be measured by established tests in the case of non-human mammals, or in the case of human mammals, by facial recognition, behavior, or speech or specific tests measuring differences in interacting time between novel and familiar animals. The terms “social memory” and “recognition memory” are used interchangeably herein.

As used herein, the term “schizophrenia” includes a condition generally described as schizophrenia or a condition having symptoms related thereto. Schizophrenia can be considered a disease having a spectrum of manifestations with various threshold levels. Symptoms of schizophrenia may appear in a range of related disorders including classical schizophrenia as well as dementia, bipolar disorder, obsessive compulsive disorder (OCD), panic disorder, phobias, acute stress disorder, adjustment disorder, agoraphobia without history of panic disorder, alcohol dependence (alcoholism), amphetamine dependence, brief psychotic disorder, cannabis dependence, cocaine dependence, cyclothymic disorder, delirium, delusional disorder, dysthymic disorder, generalized anxiety disorder, hallucinogen dependence, major depressive disorder, nicotine dependence, opioid dependence, paranoid personality disorder, Parkinson's disease, schizoaffective disorder, schizoid personality disorder, schizophreniform disorder, schizotypal personality disorder, sedative dependence, shared psychotic disorder, smoking dependence and social phobia.

The terms “vector”, “expression vector”, and “expression construct” are used interchangeably to refer to a composition of matter which can be used to deliver a nucleic acid of interest to the interior of a cell and mediate its expression within the cell. Most commonly used examples of vectors are autonomously replicating plasmids and viruses (such as, e.g., adenoviral vectors, adeno-associated virus vectors (AAV), adenoviral vectors, retroviral vectors (e.g., lentiviral vectors), Sindbis virus vectors, vaccinia virus vectors, herpes virus vectors, etc.). An expression construct can be replicated in a living cell, or it can be made synthetically. In one embodiment, an expression vector comprises a promoter operably linked to a polynucleotide which promoter controls the initiation of transcription by RNA polymerase and expression of the polynucleotide. Typical promoters for mammalian cell expression include, e.g., SV40 early promoter, CMV immediate early promoter (see, e.g., U.S. Pat. Nos. 5,168,062 and 5,385,839), mouse mammary tumor virus LTR promoter, adenovirus major late promoter (Ad MLP), herpes simplex virus promoter, murine metallothionein gene promoter, and U6 or H1 RNA pol III promoter. Non-limiting examples of promoters useful for the methods of the present invention include, e.g., Synapsin promoter (neuron specific), CamKIIa promoter (specific for excitatory neurons), CMV promoter, and β-actin promoter. These and other promoters can be obtained from commercially available plasmids, using techniques well known in the art. See, e.g., Sambrook et al., supra. Enhancer elements may be used in association with promoters to increase expression levels of the vectors. Examples include the SV40 early gene enhancer, as described in Dijkema et al., EMBO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence.

Typically, transcription terminator/polyadenylation signals will also be present in the expression vector. Examples of such sequences include, but are not limited to, those derived from SV40, as described in Sambrook et al., supra, as well as a bovine growth hormone terminator sequence (see, e.g., U.S. Pat. No. 5,122,458). Additionally, 5′-UTR sequences can be placed adjacent to the coding sequence in order to enhance expression of the same. Such sequences include UTRs which include, e.g., an Internal Ribosome Entry Site (IRES) present in the leader sequences of picomaviruses such as the encephalomyocarditis virus (EMCV) UTR (Jang et al. J. Virol. (1989) 63:1651-1660. Other useful picornavirus UTR sequences include, e.g., the polio leader sequence, hepatitis A virus leader and the hepatitis C IRES.

In certain embodiments of the invention, the cells containing nucleic acid constructs of the present invention may be identified in vitro or in vivo by including a marker in the expression vector. Such markers would confer an identifiable change to the cell permitting easy identification of cells containing the expression vector. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Fluorescent markers (e.g., green fluorescent protein (GFP), EGFP, or Dronpa), or immunologic markers can also be employed. Further examples of selectable markers are well known to one of skill in the art.

In the context of the present invention insofar as it relates to any of the disease conditions recited herein, the terms “treat”, “treatment”, and the like mean to relieve or alleviate at least one symptom associated with such condition, or to slow or reverse the progression of such condition, or to arrest, delay the onset (i.e., the period prior to clinical manifestation of a disease) and/or reduce the risk of developing or worsening a disease. Within the meaning of the present invention, the term “treat” also encompasses preventing and/or reducing a positive symptom associated with schizophrenia or 22q11 DS, such as, e.g., hallucinations, delusions, disorganized thought, or psychosis.

As used herein the term “therapeutically effective” applied to dose or amount refers to that quantity of a compound or pharmaceutical composition that is sufficient to result in a desired activity (e.g., decrease in positive symptoms associated with schizophrenia and/or 22q11DS) upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like. An appropriate “effective” amount in any individual case may be determined by one of ordinary skill in the art using routine experimentation, based upon the information provided herein.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

As used herein, the term “combination” of a composition of the invention and at least a second pharmaceutically active ingredient means at least two, but any desired combination of compounds can be delivered simultaneously or sequentially (e.g., within a 24 hour period). It is contemplated that when used to treat various diseases, the compositions and methods of the present invention can be utilized with other therapeutic methods/agents suitable for the same or similar diseases. Such other therapeutic methods/agents can be co-administered (simultaneously or sequentially) to generate additive or synergistic effects. Suitable therapeutically effective dosages for each agent may be lowered due to the additive action or synergy.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

An “individual” or “subject” or “animal”, as used herein, refers to humans, veterinary animals (e.g., cats, dogs, cows, horses, sheep, pigs, etc.) and experimental animal models of schizophrenia or 22q11 DS. In a preferred embodiment, the subject is a human.

The term “associated with” is used to encompass any correlation, co-occurrence and any cause-and-effect relationship.

The term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean within an order of magnitude, preferably within 50%, more preferably within 20%, still more preferably within 10%, even more preferably within 5%, and most preferably within 1% of a given value or range. Alternatively, particularly with respect to biological systems or processes, the term can mean within an order of magnitude, preferably within two-fold, of a value. Where particular values are described in the application and claims, unless otherwise stated, the term “about” is implicit and in this context means within an acceptable error range for the particular value.

It must be noted that, as used in this specification and the appended claims, the singular forms “a,” “an” and “the” include plural referents unless the content clearly dictates otherwise.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition. Cold Spring Harbor, N.Y.: Cold Spring Harbor Laboratory Press, 1989 (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); Ausubel, F. M. et al. (eds.). Current Protocols in Molecular Biology. John Wiley & Sons, Inc., 1994. These techniques include site directed mutagenesis as described in Kunkel, Proc. Natl. Acad. Sci. USA 82: 488-492 (1985), U.S. Pat. No. 5,071,743, Fukuoka et al., Biochem. Biophys. Res. Commun. 263: 357-360 (1999); Kim and Maas, BioTech. 28: 196-198 (2000); Parikh and Guengerich, BioTech. 24: 4 28-431 (1998); Ray and Nickoloff, BioTech. 13: 342-346 (1992); Wang et al., BioTech. 19: 556-559 (1995); Wang and Malcolm, BioTech. 26: 680-682 (1999); Xu and Gong, BioTech. 26: 639-641 (1999), U.S. Pat. Nos. 5,789,166 and 5,932,419, Hogrefe, Strategies 14. 3: 74-75 (2001), U.S. Pat. Nos. 5,702,931, 5,780,270, and 6,242,222, Angag and Schutz, Biotech. 30: 486-488 (2001), Wang and Wilkinson, Biotech. 29: 976-978 (2000), Kang et al., Biotech. 20: 44-46 (1996), Ogel and McPherson, Protein Engineer. 5: 467-468 (1992), Kirsch and Joly, Nucl. Acids. Res. 26: 1848-1850 (1998), Rhem and Hancock, J. Bacteriol. 178: 3346-3349 (1996), Boles and Miogsa, Curr. Genet. 28: 197-198 (1995), Barrenttino et al., Nuc. Acids. Res. 22: 541-542 (1993), Tessier and Thomas, Meths. Molec. Biol. 57: 229-237, and Pons et al., Meth. Molec. Biol. 67: 209-218.

The technology illustratively described herein suitably may be practiced in the absence of any element(s) not specifically disclosed herein.

The terms and expressions which have been employed are used as terms of description and not of limitation, and use of such terms and expressions do not exclude any equivalents of the features shown and described or portions thereof, and various modifications are possible within the scope of the technology claimed.

In one aspect is provided a method for treating or preventing a social memory deficit in a subject with a neuropsychiatric disease. One or more of a therapeutically effective amount of the following is administered to the subject: (i) a polypeptide encoded by 2510002D24Rik gene or a functional derivative or fragment thereof, (ii) a vector expressing the polypeptide encoded by 2510002D24Rik gene or functional derivative or fragment thereof, or (iii) an agent capable of increasing the level or activity of the polypeptide encoded by 2510002D24Rik gene. In a related aspect is provided a method for treating or preventing a social memory deficit in a subject with a neuropsychiatric disease, the method comprising administering to the subject a therapeutically effective amount of (i) a protein encoded by 2510002D24Rik gene or a functional derivative or fragment thereof, or (ii) a vector expressing said protein encoded by 2510002D24Rik gene or a functional derivative or fragment thereof.

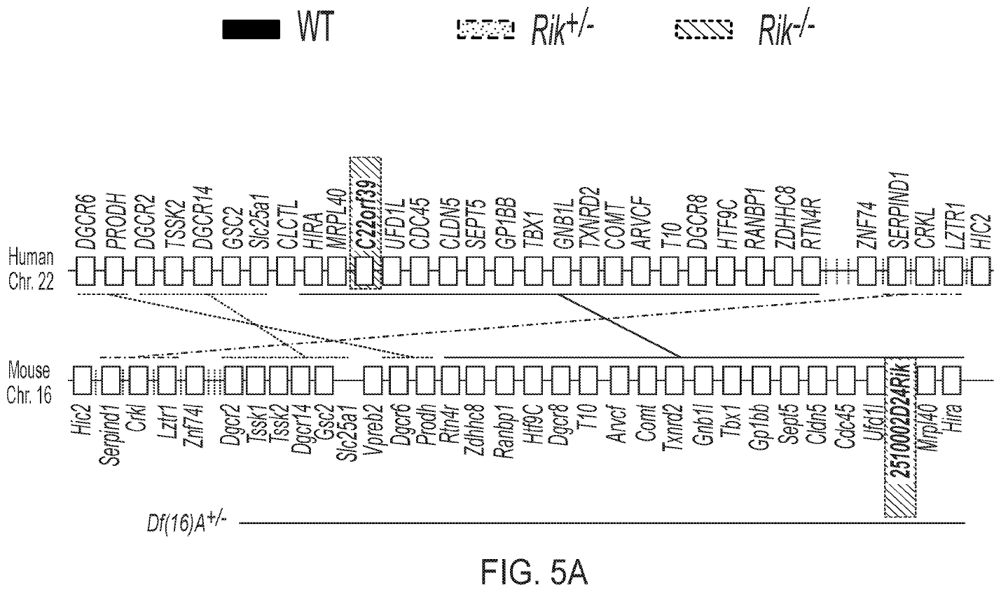

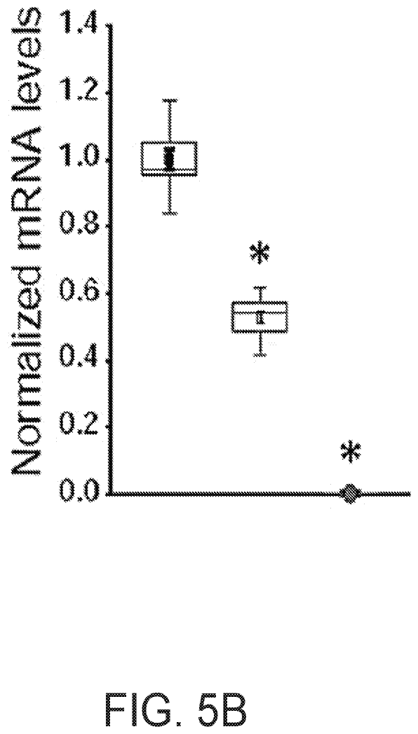

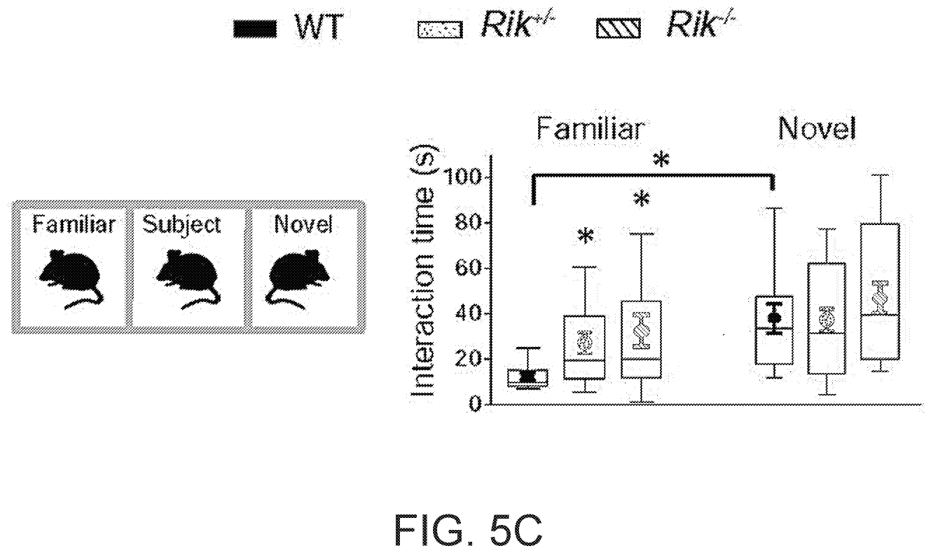

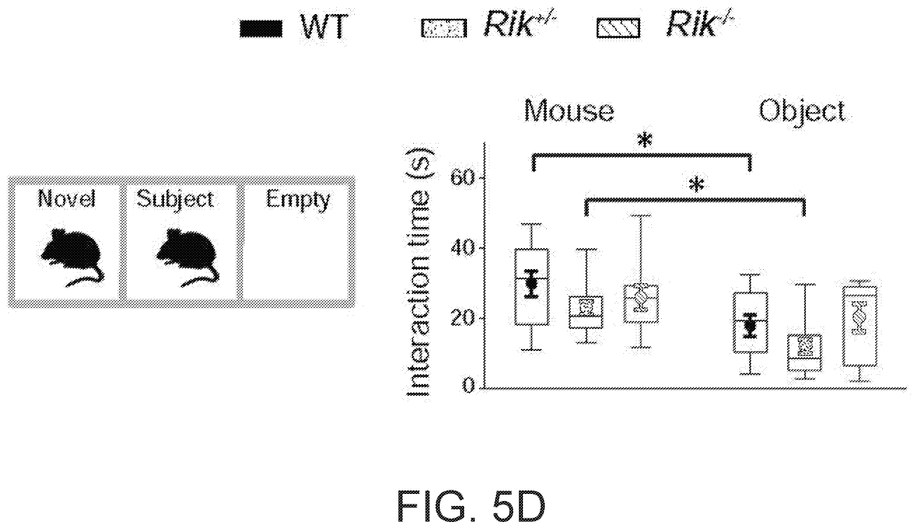

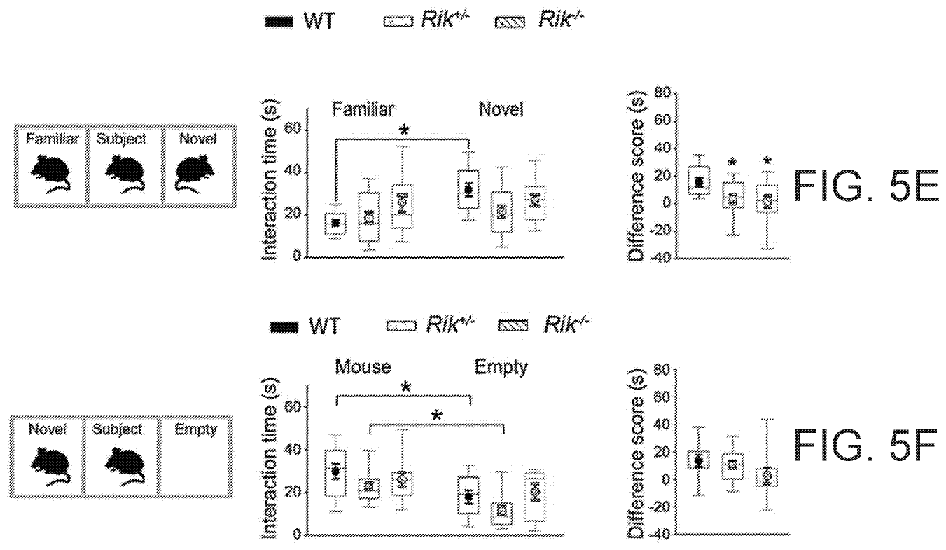

The human ortholog of 2510002D24Rik gene encodes a protein of unknown function and is mapped within the 22q11.2 genomic region (see FIG. 5A). The expression of 2510002D24Rik is strongest in the CA3 and CA2 areas of the hippocampus.¹² This application describes how 2510002D24Rik, a gene of previously unknown function, maintains metabolic stability in CA2 interneurons that are essential for social memory. Described herein are behavioral phenotypes present in mice carrying a deletion of the 2510002D24Rik gene, with heterozygous and homozygous mice referred to throughout the application as Rik^{+/-} and Rik^{-/-}, respectively). Adult but not juvenile mouse models of 22q11DS encoding a 27-gene microdeletion that encompasses the 2510002D24Rik gene have a social memory deficit associated with neuronal and synaptic dysfunctions in the CA2 area.⁸

In another aspect is provided a method for treating or preventing a social memory deficit in a subject with a neuropsychiatric disease. One or more of a therapeutically effective amount of the following is administered to the subject: (i) a polypeptide encoded by Atp23 gene or a functional derivative or fragment thereof, or (ii) a vector expressing the polypeptide encoded by Atp23 gene or functional derivative or fragment thereof, or (iii) an agent capable of increasing the level or activity of the polypeptide encoded by Atp23 gene. In a related aspect is provided a method for treating or preventing a social memory deficit in a subject with a neuropsychiatric disease, said method comprising administering to the subject a therapeutically effective amount of (i) a protein encoded by Atp23 gene or a functional derivative or fragment thereof, or (ii) a vector expressing said protein encoded by Atp23 gene or a functional derivative or fragment thereof.

In one embodiment, the present invention provides a method for treatment and/or prevention of schizophrenia in a subject in need thereof, the method comprising administering to the subject a therapeutically effective amount of (i) the polypeptide encoded by 2510002D24Rik gene or a functional derivative (including functional fragments) thereof, or (ii) a vector expressing the polypeptide encoded by 2510002D24Rik gene or functional derivative thereof, or (iii) an agent capable of increasing the level or activity of the polypeptide encoded by 2510002D24Rik gene.

In another embodiment, the present invention provides a method for treatment and/or prevention of schizophrenia in a subject in need thereof, the method comprising administering to the subject a therapeutically effective amount of (i) the polypeptide encoded by Atp23 gene or a functional derivative (including functional fragments) thereof, or (ii) a vector expressing the polypeptide encoded by Atp23 gene or functional derivative thereof, or (iii) an agent capable of increasing the level or activity of the polypeptide encoded by Atp23 gene.

Atp23 is a mitochondrial intermembrane space protein and is required for the maturation of the mitochondrially encoded F₀-subunit Atp6 and its assembly into the F₁F₀-ATP synthase complex.²⁰⁻²² Atp23 is also known as Xrcc6bp1, Atp23 metallopeptidase, and ATP synthase assembly factor homolog. A mouse Atp23 protein is comprised of the sequence: MAGAPGGGELGPAAGEPLLQRPDSGQGSPEPPAHGKPQQGFLSSLFTRDQSCPLMLQKTLD TNPYVKLLLDAMKHSGCAVNRGRHFSCEVCDGNVSGGFDASTSQIVLCENNIRNQAHMGRW THELIHAFDHCRAHVHWFTNIRHLACSEIRAASLSGDCSLVNELFRLRFGLKQHHQTCVRDRAV LSILAVRNVSREEAQKAVDEVFQTCFNDREPFGRIPHNQTYARYAHRDFQNRDRYYSNI (SEQ ID NO: 4). Another mouse Atp23 protein is comprised of the sequence:

(SEQ ID NO: 5)
MAGAPGGGELGPAAGEPLLQRPDSGQGSPEPPAHGKPQQGFLSSLFTRDQ
SCPLMLQKTLDTNPYVKLLLDAMKHSGCAVNRGRHFSCEVCDGNVSGGFD
ASTSQIVLCENNIRNQAHMGRVVTHELIHAFDHCRAHVHWFTNIRHLACS
EIRAASLSGDCSLVNELFRLRFGLKQHHQIETSCVSRPAMNSQSCLGLVS
A.
The human Atp23 protein is comprised of the
sequence:
(SEQ ID NO: 2)
MAGAPDERRRGPAAGEQLQQQHVSCQVFPERLAQGNPQQGFFSSFFTSNQ
KCQLRLLKTLETNPYVKLLLDAMKHSGCAVNKDRHFSCEDCNGNVSGGFD
ASTSQIVLCQNNIHNQAHMNRVVTHELIHAFDHCRAHVDWFTNIRHLACS
EVRAANLSGDCSLVNEIFRLHFGLKQHHQTCVRDRATLSILAVRNISKEV
AKKAVDEVFESCFNDHEPFGRIPHNKTYARYAHRDFENRDRYYSNI

In another embodiment, the invention provides a method for treatment of 22q11 deletion syndrome in a subject in need thereof, the method comprising administering to the subject a therapeutically effective amount of (i) the polypeptide encoded by 2510002D24Rik gene or a functional derivative (including functional fragments) thereof, or (ii) a vector expressing the polypeptide encoded by 2510002D24Rik gene or functional derivative thereof, or (iii) an agent capable of increasing the level or activity of the polypeptide encoded by 2510002D24Rik gene.

In another embodiment, the invention provides a method for treatment of 22q11 deletion syndrome in a subject in need thereof, the method comprising administering to the subject a therapeutically effective amount of (i) the polypeptide encoded by Atp23 gene or a functional derivative (including functional fragments) thereof, or (ii) a vector expressing the polypeptide encoded by Atp23 gene or functional derivative thereof, or (iii) an agent capable of increasing the level or activity of the polypeptide encoded by Atp23 gene.

In yet another embodiment, the invention provides a method for treatment of a positive symptom of schizophrenia in a subject in need thereof, the method comprising administering to the subject a therapeutically effective amount of (i) the polypeptide encoded by 2510002D24Rik gene or a functional derivative (including functional fragments) thereof, or (ii) a vector expressing the polypeptide encoded by 2510002D24Rik gene or functional derivative thereof, or (iii) an agent capable of increasing the level or activity of the polypeptide encoded by 2510002D24Rik gene.

In yet another embodiment, the invention provides a method for treatment of a positive symptom of schizophrenia in a subject in need thereof, the method comprising administering to the subject a therapeutically effective amount of (i) the polypeptide encoded by Atp23 gene or a functional derivative (including functional fragments) thereof, or (ii) a vector expressing the polypeptide encoded by Atp23 gene or functional derivative thereof, or (iii) an agent capable of increasing the level or activity of the polypeptide encoded by Atp23 gene.

In yet another embodiment, the invention provides a method for treatment of a mental disorder caused by, or aggravated by, a social memory deficit. The method comprises administering to the subject a therapeutically effective amount of (i) the polypeptide encoded by 2510002D24Rik gene or a functional derivative (including functional fragments) thereof, or (ii) a vector expressing the polypeptide encoded by 2510002D24Rik gene or functional derivative thereof, or (iii) an agent capable of increasing the level or activity of the polypeptide encoded by 2510002D24Rik gene.

In yet another embodiment, the invention provides a method for treatment of a mental disorder caused by, or aggravated by, a social memory deficit. The method comprises administering to the subject a therapeutically effective amount of (i) the polypeptide encoded by Atp23 gene or a functional derivative (including functional fragments) thereof, or (ii) a vector expressing the polypeptide encoded by Atp23 gene or functional derivative thereof, or (iii) an agent capable of increasing the level or activity of the polypeptide encoded by Atp23 gene.

In various embodiments of the above aspects and embodiments, the administration results in replenishing Atp23 in CA2 interneurons of the subject. In some embodiments, the Atp23 in the CA2 interneurons is replenished to a level of Atp23 found in the CA2 interneurons of a healthy subject without any deficiency in social memory. In some embodiments, the Atp23 in the CA2 interneurons is replenished to a level of Atp23 found in the CA2 interneurons of a normal subject. In some embodiments, the Atp23 in the CA2 interneurons is replenished to a level sufficient to overcome a social recognition deficit. In various embodiments, the CA2 interneurons are parvalbumin (PV)-positive interneurons.

In various embodiments of the above aspects and embodiments, the administration results in replenishing Atp23 in CA2 area of the hippocampus of the subject. In some embodiments, the Atp23 in the CA2 area of the hippocampus is replenished to a level of Atp23 found in the CA2 area of the hippocampus of a healthy subject without any deficiency in social memory. In some embodiments, the Atp23 in the CA2 area of the hippocampus is replenished to a level of Atp23 found in the CA2 area of the hippocampus of a normal subject. In some embodiments, the Atp23 in the CA2 area of the hippocampus is replenished to a level sufficient to overcome a social recognition deficit.

In various embodiments of the above aspects and embodiments, the neuropsychiatric disease is a schizophrenia spectrum disorder. In various embodiments of the above aspects and embodiments, the neuropsychiatric disease is an autism spectrum disorder. In some embodiments, the neuropsychiatric disease is selected from schizophrenia, 22q11 deletion syndrome, attention-deficit hyperactivity disorder, generalized anxiety disorder, obsessive-compulsive disorder and autism spectrum disorders (ASD).

In any of the above aspects and embodiments, any viral vector can be used that is capable of accepting the coding sequences for 2510002D24Rik and a polypeptide or protein encoded by Atp23. For example, vectors derived from adenovirus (AV), adeno-associated virus (AAV), retroviruses (e.g., lentiviruses (LV), Rhabdoviruses, murine leukemia virus), Sindbis virus, herpes virus, and the like. The tropism of the viral vectors can be modified by pseudotyping the vectors with envelope proteins or other surface antigens from other viruses, or by substituting different viral capsid proteins, as appropriate. For example, lentiviral vectors of the invention can be pseudotyped with surface proteins from vesicular stomatitis virus (VSV), rabies, Ebola, Mokola, and the like. AAV vectors of the invention can be made to specifically target certain cells or tissues by engineering the vectors to express certain capsid protein serotypes. Currently, there are several AAV serotypes available that can be used for tissue enrichment based on natural tropism toward specific cell types and interaction between different cellular receptors and serotypes. For example, an AAV vector expressing a serotype 2 capsid on a serotype 2 genome is called AAV 2/2. This serotype 2 capsid gene in the AAV 2/2 vector can be replaced by a serotype 5 capsid gene to produce an AAV 2/5 vector. Techniques for constructing AAV vectors which express different capsid protein serotypes are within the skill in the art; see, e.g., Rabinowitz J. E. et al. (2002), J Virol 76:791801. A method for constructing the recombinant AV vector, and a method for delivering the vector into target cells, are also described in Xia et al. (2002), Nat. Biotech. 20:1006-1010.

In some embodiments, the vector is selected from the group consisting of adeno-associated virus (AAV) vectors, adenovirus vectors, retrovirus vectors (e.g., lentivirus vectors), Sindbis virus vectors, vaccinia virus vectors, and herpes virus vectors. In some embodiments, the vector is an AAV vector. The AAV vector may have a capsid from a serotype selected from AAV1, AAV2, AAV5, AAV8, or AAV9.

Alternatively, the 2510002D24Rik and the polypeptide encoded by Atp23 can be expressed from recombinant circular or linear DNA plasmids using any suitable promoter, including inducible/regulatable promoters. In some embodiments, 2510002D24Rik is expressed from a DNA plasmid. In some embodiments, one or more peptides encoded by Atp23 is expressed from a DNA plasmid. In some embodiments, both (i) 2510002D24Rik and (ii) one or more peptides encoded by Atp23 are expressed from the same DNA plasmid. An IRES sequence may be placed upstream of one of the (i) 2510002D24Rik and (ii) one or more peptides encoded by Atp23.

In various embodiments, the expression of the peptide or functional derivative or fragment thereof in the vector is controlled by an fsst promoter. In various embodiments, the expression of the peptide or functional derivative or fragment thereof in the vector is controlled by a Synapsin promoter. In various embodiments, the expression of the peptide or functional derivative or fragment thereof in the vector is controlled by a CMV promoter. In various embodiments, the expression of the peptide or functional derivative or fragment thereof in the vector is controlled by a β-actin promoter. In various embodiments, the expression of the peptide or functional derivative or fragment thereof in the vector is controlled by an hDlx promoter. In various embodiments, the expression of the peptide or functional derivative or fragment thereof in the vector is controlled by an mDlx promoter. In various embodiments, the expression of the peptide or functional derivative or fragment thereof in the vector is controlled by a CamKIIa promoter.

The expression of the protein or functional derivative or fragment thereof in the vector can be controlled by a pan-GABAergic interneuron promoter. Exemplary pan-GABAergic interneuron promoters include, but are not limited to, Dlx, the promoter of GABAergic marker GAD67, the promoter of VGAT (SLC32A1), the promoter of GAD1, and the promoter of SLC6A1.

Various modes of administration may be undertaken. In some embodiments, the administration is via injection into the CA2 area of the hippocampus of the subject. Alternatively, on some embodiments administration is via a transcranial surgical injection. Injection may be performed using various syringes. Exemplary syringes that may be used include, but are not limited to, Hamilton syringes. Ultrathin needles designed for injecting materials into the brain may also be used. Various micropump devices may also be used with syringes and needles. Various other apparatus for intracerebral drug administration can be used, such as those described in International Patent Publication Nos. WO2019/088690 and WO2009/144287, both of which are incorporated by reference herein in their entireties.

In some embodiments, administration is systemic. Systemic administration can result in delivery to essentially the entire body of an individual. Routes of administration suitable for or treating disorders disclosed herein also include both central and peripheral administration. Central administration may result in delivery of a combination to essentially the central nervous system of the individual and includes, e.g., nasal administration, intrathecal administration, epidural administration as well as a cranial injection or implant. Peripheral administration can result in delivery of a compound or a combination to essentially any area of an individual outside of the central nervous system and encompasses any route of administration other than direct administration to the spine or brain. The actual route of administration of a compound or a combination disclosed herein used can be determined by a person of ordinary skill in the art by taking into account factors, including, without limitation, the type of disorder, the cause of the nervous system disorder, the severity of the nervous system disorder, the duration of treatment desired, the degree of relief desired, the duration of relief desired, the particular compound used, the rate of excretion of the compound used, the pharmacodynamics of the compound or combination used, the nature of the other compounds to be included in the combination, the particular route of administration, the particular characteristics, history and risk factors of the individual, such as, e.g., age, weight, general health and the like, the response of the individual to the treatment, or any combination thereof. An effective dosage amount of a compound disclosed herein can thus readily be determined by the person of ordinary skill in the art considering all criteria and utilizing his best judgment on the individual's behalf.

In various embodiments, the mode of administration is intranasal.

Liquid formulations suitable for injection or for nasal sprays may comprise physiologically acceptable sterile aqueous or nonaqueous solutions, dispersions, suspensions or emulsions and sterile powders for reconstitution into sterile injectable solutions or dispersions. Formulations suitable for nasal administration may comprise physiologically acceptable sterile aqueous or nonaqueous solutions, dispersions, suspensions or emulsions. Examples of suitable aqueous and nonaqueous carriers, diluents, solvents or vehicles include water, ethanol, polyols (propylene glycol, polyethyleneglycol (PEG), glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

Pharmaceutical formulations suitable for administration by inhalation include fine particle dusts or mists, which may be generated by means of various types of metered, dose pressurized aerosols, nebulizers, or insufflators.

A therapeutically effective amount of the compound may be administered in a dosing regimen that comprises a single unit dose or multiple unit doses. For any particular therapeutic peptide or vector, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including, but not limited to, the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific fusion protein employed; and the duration of the treatment.

In some embodiments, the therapeutically effective dose ranges from about 0.005 mg/kg brain weight to 500 mg/kg brain weight, e.g., from about 0.005 mg/kg brain weight to 400 mg/kg brain weight, from about 0.005 mg/kg brain weight to 300 mg/kg brain weight, from about 0.005 mg/kg brain weight to 200 mg/kg brain weight, from about 0.005 mg/kg brain weight to 100 mg/kg brain weight, from about 0.005 mg/kg brain weight to 90 mg/kg brain weight, from about 0.005 mg/kg brain weight to 80 mg/kg brain weight, from about 0.005 mg/kg brain weight to 70 mg/kg brain weight, from about 0.005 mg/kg brain weight to 60 mg/kg brain weight, from about 0.005 mg/kg brain weight to 50 mg/kg brain weight, from about 0.005 mg/kg brain weight to 40 mg/kg brain weight, from about 0.005 mg/kg brain weight to 30 mg/kg brain weight, from about 0.005 mg/kg brain weight to 25 mg/kg brain weight, from about 0.005 mg/kg brain weight to 20 mg/kg brain weight, from about 0.005 mg/kg brain weight to 15 mg/kg brain weight, from about 0.005 mg/kg brain weight to 10 mg/kg brain weight.

In some embodiments, the therapeutically effective dose is greater than about 0.1 mg/kg brain weight, greater than about 0.5 mg/kg brain weight, greater than about 1.0 mg/kg brain weight, greater than about 3 mg/kg brain weight, greater than about 5 mg/kg brain weight, greater than about 10 mg/kg brain weight, greater than about 15 mg/kg brain weight, greater than about 20 mg/kg brain weight, greater than about 30 mg/kg brain weight, greater than about 40 mg/kg brain weight, greater than about 50 mg/kg brain weight, greater than about 60 mg/kg brain weight, greater than about 70 mg/kg brain weight, greater than about 80 mg/kg brain weight, greater than about 90 mg/kg brain weight, greater than about 100 mg/kg brain weight, greater than about 150 mg/kg brain weight, greater than about 200 mg/kg brain weight, greater than about 250 mg/kg brain weight, greater than about 300 mg/kg brain weight, greater than about 350 mg/kg brain weight, greater than about 400 mg/kg brain weight, greater than about 450 mg/kg brain weight, greater than about 500 mg/kg brain weight.

In some embodiments, the therapeutically effective dose may also be defined by mg/kg body weight. As one skilled in the art would appreciate, the brain weights and body weights can be correlated. Dekaban A S. “Changes in brain weights during the span of human life: relation of brain weights to body heights and body weights,” Ann Neurol 1978; 4:345-56.

Also provided is a container comprising a single dosage form of a stable formulation in various embodiments described herein. In some embodiments, the container is selected from an ampule, a vial, a bottle, a cartridge, a reservoir, a lyo-ject, or a pre-filled syringe. In some embodiments, the container is a prefilled syringe. In some embodiments, the pre-filled syringe is selected from borosilicate glass syringes with baked silicone coating, borosilicate glass syringes with sprayed silicone, or plastic resin syringes without silicone. In some embodiments, the stable formulation is present in a volume of less than about 50 mL (e.g., less than about 45 mL, 40 mL, 35 mL, 30 mL, 25 mL, 20 mL, 15 mL, 10 mL, 5 mL, 4 mL, 3 mL, 2.5 mL, 2.0 mL, 1.5 mL, 1.0 mL, or 0.5 mL). In some embodiments, the stable formulation is present in a volume of less than about 3.0 mL.

Also provided are kits or other articles of manufacture which contains the formulation of the present invention and provides instructions for its reconstitution (if lyophilized) and/or use. Kits or other articles of manufacture may include a container, an IDDD, a catheter and any other articles, devices or equipment useful in intrathecal administration and associated surgery. Suitable containers include, for example, bottles, vials, syringes (e.g., pre-filled syringes), ampules, cartridges, reservoirs, or lyo-jects. The container may be formed from a variety of materials such as glass or plastic. In some embodiments, a container is a pre-filled syringe. Suitable pre-filled syringes include, but are not limited to, borosilicate glass syringes with baked silicone coating, borosilicate glass syringes with sprayed silicone, or plastic resin syringes without silicone.

The container may hold formulations and further comprise a label on, or associated with, the container that may indicate directions for reconstitution and/or use. For example, the label may indicate that the formulation is reconstituted to protein concentrations as described above. The label may further indicate that the formulation is useful or intended for, for example, intrathecal administration. In some embodiments, a container may contain a single dose of a stable formulation containing a therapeutic agent (e.g., a replacement enzyme). In various embodiments, a single dose of the stable formulation is present in a volume of less than about 15 ml, 10 ml, 5.0 ml, 4.0 ml, 3.5 ml, 3.0 ml, 2.5 ml, 2.0 ml, 1.5 ml, 1.0 ml, or 0.5 ml. Alternatively, a container holding the formulation may be a multi-use vial, which allows for repeat administrations (e.g., from 2-6 administrations) of the formulation. Kits or other articles of manufacture may further include a second container comprising a suitable diluent (e.g., BWFI, saline, buffered saline). Upon mixing of the diluent and the formulation, the final protein concentration in the reconstituted formulation will generally be at least 1 mg/ml (e.g., at least 5 mg/ml, at least 10 mg/ml, at least 25 mg/ml, at least 50 mg/ml, at least 75 mg/ml, at least 100 mg/ml). Kits or other articles of manufacture may further include other materials desirable from a commercial and user standpoint, including other buffers, diluents, filters, needles, IDDDs, catheters, syringes, and package inserts with instructions for use.

In various embodiments, the protein encoded by 2510002D24Rik gene comprises the amino acid sequence which has at least 80% sequence identity to SEQ ID NO: 1. In various embodiments, the protein encoded by 2510002D24Rik gene comprises the amino acid sequence which has at least 83% sequence identity to SEQ ID NO: 1. In various embodiments, the protein encoded by 2510002D24Rik gene comprises the amino acid sequence which has at least 85% sequence identity to SEQ ID NO: 1. In various embodiments, the protein encoded by 2510002D24Rik gene comprises the amino acid sequence which has at least 87% sequence identity to SEQ ID NO: 1. In various embodiments, the protein encoded by 2510002D24Rik gene comprises the amino acid sequence which has at least 90% sequence identity to SEQ ID NO: 1. In various embodiments, the protein encoded by 2510002D24Rik gene comprises the amino acid sequence which has at least 91% sequence identity to SEQ ID NO: 1. In various embodiments, the protein encoded by 2510002D24Rik gene comprises the amino acid sequence which has at least 92% sequence identity to SEQ ID NO: 1. In various embodiments, the protein encoded by 2510002D24Rik gene comprises the amino acid sequence which has at least 93% sequence identity to SEQ ID NO: 1. In various embodiments, the protein encoded by 2510002D24Rik gene comprises the amino acid sequence which has at least 94% sequence identity to SEQ ID NO: 1. In various embodiments, the protein encoded by 2510002D24Rik gene comprises the amino acid sequence which has at least 95% sequence identity to SEQ ID NO: 1. In various embodiments, the protein encoded by 2510002D24Rik gene comprises the amino acid sequence which has at least 96% sequence identity to SEQ ID NO: 1. In various embodiments, the protein encoded by 2510002D24Rik gene comprises the amino acid sequence which has at least 97% sequence identity to SEQ ID NO: 1. In various embodiments, the protein encoded by 2510002D24Rik gene comprises the amino acid sequence which has at least 98% sequence identity to SEQ ID NO: 1. In various embodiments, the protein encoded by 2510002D24Rik gene comprises the amino acid sequence which has at least 99% sequence identity to SEQ ID NO: 1. In some embodiments, the protein encoded by 2510002D24Rik gene comprises the amino acid sequence SEQ ID NO: 1. In some embodiments, the protein encoded by 2510002D24Rik gene consists of the amino acid sequence SEQ ID NO: 1.

In various embodiments, the protein encoded by 2510002D24Rik gene comprises the amino acid sequence which has at least 80% sequence identity to SEQ ID NO: 3. In various embodiments, the protein encoded by 2510002D24Rik gene comprises the amino acid sequence which has at least 83% sequence identity to SEQ ID NO: 3. In various embodiments, the protein encoded by 2510002D24Rik gene comprises the amino acid sequence which has at least 85% sequence identity to SEQ ID NO: 3. In various embodiments, the protein encoded by 2510002D24Rik gene comprises the amino acid sequence which has at least 87% sequence identity to SEQ ID NO: 3. In various embodiments, the protein encoded by 2510002D24Rik gene comprises the amino acid sequence which has at least 90% sequence identity to SEQ ID NO: 3. In various embodiments, the protein encoded by 2510002D24Rik gene comprises the amino acid sequence which has at least 91% sequence identity to SEQ ID NO: 3. In various embodiments, the protein encoded by 2510002D24Rik gene comprises the amino acid sequence which has at least 92% sequence identity to SEQ ID NO: 3. In various embodiments, the protein encoded by 2510002D24Rik gene comprises the amino acid sequence which has at least 93% sequence identity to SEQ ID NO: 3. In various embodiments, the protein encoded by 2510002D24Rik gene comprises the amino acid sequence which has at least 94% sequence identity to SEQ ID NO: 3. In various embodiments, the protein encoded by 2510002D24Rik gene comprises the amino acid sequence which has at least 95% sequence identity to SEQ ID NO: 3. In various embodiments, the protein encoded by 2510002D24Rik gene comprises the amino acid sequence which has at least 96% sequence identity to SEQ ID NO: 3. In various embodiments, the protein encoded by 2510002D24Rik gene comprises the amino acid sequence which has at least 97% sequence identity to SEQ ID NO: 3. In various embodiments, the protein encoded by 2510002D24Rik gene comprises the amino acid sequence which has at least 98% sequence identity to SEQ ID NO: 3. In various embodiments, the protein encoded by 2510002D24Rik gene comprises the amino acid sequence which has at least 99% sequence identity to SEQ ID NO: 3. In some embodiments, the protein encoded by 2510002D24Rik gene comprises the amino acid sequence SEQ ID NO: 3. In some embodiments, the protein encoded by 2510002D24Rik gene consists of the amino acid sequence SEQ ID NO: 3.

In various embodiments, the protein encoded by Atp23 gene comprises the amino acid sequence which has at least 80% sequence identity to SEQ ID NO: 2. In various embodiments, the protein encoded by Atp23 gene comprises the amino acid sequence which has at least 83% sequence identity to SEQ ID NO: 2. In various embodiments, the protein encoded by Atp23 gene comprises the amino acid sequence which has at least 85% sequence identity to SEQ ID NO: 2. In various embodiments, the protein encoded by Atp23 gene comprises the amino acid sequence which has at least 87% sequence identity to SEQ ID NO: 2. In various embodiments, the protein encoded by Atp23 gene comprises the amino acid sequence which has at least 90% sequence identity to SEQ ID NO: 2. In various embodiments, the protein encoded by Atp23 gene comprises the amino acid sequence which has at least 91% sequence identity to SEQ ID NO: 2. In various embodiments, the protein encoded by Atp23 gene comprises the amino acid sequence which has at least 92% sequence identity to SEQ ID NO: 2. In various embodiments, the protein encoded by Atp23 gene comprises the amino acid sequence which has at least 93% sequence identity to SEQ ID NO: 2. In various embodiments, the protein encoded by Atp23 gene comprises the amino acid sequence which has at least 94% sequence identity to SEQ ID NO: 2. In various embodiments, the protein encoded by Atp23 gene comprises the amino acid sequence which has at least 95% sequence identity to SEQ ID NO: 2. In various embodiments, the protein encoded by Atp23 gene comprises the amino acid sequence which has at least 96% sequence identity to SEQ ID NO: 2. In various embodiments, the protein encoded by Atp23 gene comprises the amino acid sequence which has at least 97% sequence identity to SEQ ID NO: 2. In various embodiments, the protein encoded by Atp23 gene comprises the amino acid sequence which has at least 98% sequence identity to SEQ ID NO: 2. In various embodiments, the protein encoded by Atp23 gene comprises the amino acid sequence which has at least 99% sequence identity to SEQ ID NO: 2. In some embodiments, the protein encoded by Atp23 gene comprises the amino acid sequence SEQ ID NO: 2. In some embodiments, the protein encoded by Atp23 gene consists of the amino acid sequence SEQ ID NO: 2.

In various embodiments, the protein encoded by Atp23 gene comprises the amino acid sequence which has at least 80% sequence identity to SEQ ID NO: 4. In various embodiments, the protein encoded by Atp23 gene comprises the amino acid sequence which has at least 83% sequence identity to SEQ ID NO: 4. In various embodiments, the protein encoded by Atp23 gene comprises the amino acid sequence which has at least 85% sequence identity to SEQ ID NO: 4. In various embodiments, the protein encoded by Atp23 gene comprises the amino acid sequence which has at least 87% sequence identity to SEQ ID NO: 4. In various embodiments, the protein encoded by Atp23 gene comprises the amino acid sequence which has at least 90% sequence identity to SEQ ID NO: 4. In various embodiments, the protein encoded by Atp23 gene comprises the amino acid sequence which has at least 91% sequence identity to SEQ ID NO: 4. In various embodiments, the protein encoded by Atp23 gene comprises the amino acid sequence which has at least 92% sequence identity to SEQ ID NO: 4. In various embodiments, the protein encoded by Atp23 gene comprises the amino acid sequence which has at least 93% sequence identity to SEQ ID NO: 4. In various embodiments, the protein encoded by Atp23 gene comprises the amino acid sequence which has at least 94% sequence identity to SEQ ID NO: 4. In various embodiments, the protein encoded by Atp23 gene comprises the amino acid sequence which has at least 95% sequence identity to SEQ ID NO: 4. In various embodiments, the protein encoded by Atp23 gene comprises the amino acid sequence which has at least 96% sequence identity to SEQ ID NO: 4. In various embodiments, the protein encoded by Atp23 gene comprises the amino acid sequence which has at least 97% sequence identity to SEQ ID NO: 4. In various embodiments, the protein encoded by Atp23 gene comprises the amino acid sequence which has at least 98% sequence identity to SEQ ID NO: 4. In various embodiments, the protein encoded by Atp23 gene comprises the amino acid sequence which has at least 99% sequence identity to SEQ ID NO: 4. In some embodiments, the protein encoded by Atp23 gene comprises the amino acid sequence SEQ ID NO: 4. In some embodiments, the protein encoded by Atp23 gene consists of the amino acid sequence SEQ ID NO: 4.

In various embodiments, the protein encoded by Atp23 gene comprises the amino acid sequence which has at least 80% sequence identity to SEQ ID NO: 5. In various embodiments, the protein encoded by Atp23 gene comprises the amino acid sequence which has at least 83% sequence identity to SEQ ID NO: 5. In various embodiments, the protein encoded by Atp23 gene comprises the amino acid sequence which has at least 85% sequence identity to SEQ ID NO: 5. In various embodiments, the protein encoded by Atp23 gene comprises the amino acid sequence which has at least 87% sequence identity to SEQ ID NO: 5. In various embodiments, the protein encoded by Atp23 gene comprises the amino acid sequence which has at least 90% sequence identity to SEQ ID NO: 5. In various embodiments, the protein encoded by Atp23 gene comprises the amino acid sequence which has at least 91% sequence identity to SEQ ID NO: 5. In various embodiments, the protein encoded by Atp23 gene comprises the amino acid sequence which has at least 92% sequence identity to SEQ ID NO: 5. In various embodiments, the protein encoded by Atp23 gene comprises the amino acid sequence which has at least 93% sequence identity to SEQ ID NO: 5. In various embodiments, the protein encoded by Atp23 gene comprises the amino acid sequence which has at least 94% sequence identity to SEQ ID NO: 5. In various embodiments, the protein encoded by Atp23 gene comprises the amino acid sequence which has at least 95% sequence identity to SEQ ID NO: 5. In various embodiments, the protein encoded by Atp23 gene comprises the amino acid sequence which has at least 96% sequence identity to SEQ ID NO: 5. In various embodiments, the protein encoded by Atp23 gene comprises the amino acid sequence which has at least 97% sequence identity to SEQ ID NO: 5. In various embodiments, the protein encoded by Atp23 gene comprises the amino acid sequence which has at least 98% sequence identity to SEQ ID NO: 5. In various embodiments, the protein encoded by Atp23 gene comprises the amino acid sequence which has at least 99% sequence identity to SEQ ID NO: 5. In some embodiments, the protein encoded by Atp23 gene comprises the amino acid sequence SEQ ID NO: 5. In some embodiments, the protein encoded by Atp23 gene consists of the amino acid sequence SEQ ID NO: 5.

In various embodiments of the above, the subject is human. In certain embodiments, the subject is an adult.

In another aspect is provided a pharmaceutical composition comprising a protein encoded by 2510002D24Rik gene or a functional derivative or fragment thereof and a pharmaceutically acceptable carrier or excipient. In a related aspect is provided a pharmaceutical composition comprising a vector encoding a protein encoded by 2510002D24Rik gene or a functional derivative or fragment thereof and a pharmaceutically acceptable carrier or excipient. In yet another aspect is provided a pharmaceutical composition comprising a protein encoded by Atp23 gene or a functional derivative or fragment thereof and a pharmaceutically acceptable carrier or excipient. In a related aspect is provided a pharmaceutical composition comprising a vector encoding a protein encoded by Atp23 gene or a functional derivative or fragment thereof and a pharmaceutically acceptable carrier or excipient.

Various vectors may be used. Exemplary vectors include, but are not limited to, adeno-associated virus (AAV) vectors, retrovirus vectors (e.g., lentivirus vectors), adenovirus vectors, Sindbis virus vectors, vaccinia virus vectors, and herpes virus vectors. Lentivirus and adeno-associated virus (AAV) vectors can provide for efficiency and stability when used for gene therapy. The vector may be an AAV vector. Recombinant adeno-associated virus (AAV) vectors are a promising alternative gene delivery system based on the defective and nonpathogenic parvovirus adeno-associated type 2 virus. The AAV vectors may be derived from a plasmid that retains only the AAV 145 bp inverted terminal repeats flanking the transgene expression cassette. Efficient gene transfer and stable transgene delivery due to integration into the genomes of the transduced cell may be provided by this vector system. (Wagner, et al. (1998) Lancet 351(9117):1702-1703; Keams, et al. (1996) Gene Ther. 9:748-55). Other AAV serotypes, including AAV1, AAV3, AAV4, AAV5, AAV6, AAV8AAV 8.2, AAV9, and AAV rh10 and pseudotyped AAV such as AAV2/8, AAV2/5 and AAV2/6 can also be used. In certain embodiments, the AAV vector has a capsid from a serotype selected from AAV1, AAV2, AAV5, AAV8, and AAV9.

Various retroviral vectors may also be used. In certain embodiments, the retrovirus vector is a lentivirus vector. Recombinant lentiviral vectors may advantageously deliver and express peptides in both dividing and non-dividing mammalian cells.

In various embodiments, in the vector the sequence encoding the protein encoded by 2510002D24Rik gene or functional derivative or fragment thereof is operably linked to a promoter selected from the group consisting of fsst promoter, hDlx promoter, mDlx promoter, Synapsin promoter, CMV promoter, β-actin promoter, and CamKIIa promoter. In related embodiments, in the vector the sequence encoding the protein encoded by Atp23 gene or functional derivative or fragment thereof is operably linked to a promoter selected from the group consisting of fsst promoter, hDlx promoter, mDlx promoter, Synapsin promoter, CMV promoter, β-actin promoter, and CamKIIa promoter.

In the vector, the sequence encoding the protein encoded by 2510002D24Rik gene or functional derivative or fragment thereof may be operably linked to a pan-GABAergic interneuron promoter. In the vector, the sequence encoding the protein encoded by Atp23 gene or functional derivative or fragment thereof may be operably linked to a pan-GABAergic interneuron promoter.

In the pharmaceutical composition, the protein encoded by 2510002D24Rik gene may comprises the amino acid sequence which has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 1. In some embodiments, the protein encoded by 2510002D24Rik gene comprises the amino acid sequence having 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 1. In certain embodiments, the protein encoded by 2510002D24Rik gene comprises the amino acid sequence SEQ ID NO: 1. In certain embodiments, the protein encoded by 2510002D24Rik gene consists of the amino acid sequence SEQ ID NO: 1.

In the pharmaceutical composition, the protein encoded by 2510002D24Rik gene may comprises the amino acid sequence which has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 3. In some embodiments, the protein encoded by 2510002D24Rik gene comprises the amino acid sequence having 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 3. In certain embodiments, the protein encoded by 2510002D24Rik gene comprises the amino acid sequence SEQ ID NO: 3. In certain embodiments, the protein encoded by 2510002D24Rik gene consists of the amino acid sequence SEQ ID NO: 3.

In the pharmaceutical composition, the protein encoded by Atp23 gene may comprises the amino acid sequence which has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 2. In some embodiments, the protein encoded by Atp23 gene comprises the amino acid sequence having 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 2. In certain embodiments, the protein encoded by Atp23 gene comprises the amino acid sequence SEQ ID NO: 2. In certain embodiments, the protein encoded by Atp23 gene consists of the amino acid sequence SEQ ID NO: 2.

In the pharmaceutical composition, the protein encoded by Atp23 gene may comprises the amino acid sequence which has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 4. In some embodiments, the protein encoded by Atp23 gene comprises the amino acid sequence having 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 4. In certain embodiments, the protein encoded by Atp23 gene comprises the amino acid sequence SEQ ID NO: 4. In certain embodiments, the protein encoded by Atp23 gene consists of the amino acid sequence SEQ ID NO: 4.

In the pharmaceutical composition, the protein encoded by Atp23 gene may comprises the amino acid sequence which has at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 5. In some embodiments, the protein encoded by Atp23 gene comprises the amino acid sequence having 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 5. In certain embodiments, the protein encoded by Atp23 gene comprises the amino acid sequence SEQ ID NO: 5. In certain embodiments, the protein encoded by Atp23 gene consists of the amino acid sequence SEQ ID NO: 5.

In various embodiments, the pharmaceutical composition is formulated for injection into hippocampus. In various embodiments, the pharmaceutical composition is formulated for transcranial surgical injection.

### Examples

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Materials and Methods

### Animals

Mature (16-28 weeks) mice were used for the experiments. All experiments were carried out using age- and sex-matched mice, except for social behavior experiments, where only age-matched male mice were used. All mouse strains used herein were back-crossed onto the C57BL/6J genetic background for at least 5 generations. The care and use of animals were reviewed and approved by the Institutional Animal Care and Use Committee at St. Jude Children's Research Hospital.

### Generation of 2510002D24Rik Knock-Out Mice

The 2510002D24Rik knock-out (KO) (Rik^{+/-} and Rik^{-/-}) mice were produced from the embryonic stem (ES) cell clone (12035A-B12, 12035A-E11), which was produced by Regeneron Pharmaceuticals, Inc. (Tarrytown, N.Y.) and generated into live mice by KOMP (www.komp.org) and the Mouse Biology Program (www.mousebiology.org) at the University of California, Davis. Rik^{+/-} mice were generated using a targeting vector to disrupt the gene downstream of the start codon. Mice were then crossed with Ella-cre mice (Jackson Labs, Cat No. 003724) expressing a germline-specific cre recombinase to delete the neomycin cassette. Mice were genotyped, as previously described¹², using PCR and the following primers: WT Forward Primer: ACC ATG TGA ATC TAC TGC CTG AGG G (SEQ ID NO: 6), WT Reverse Primer: TAT GTG GGT GAA TGC CTG TAG TCC C (SEQ ID NO: 7), and Rik Forward Primer: GCT CAC CTA CAC TCT GTG TAT G (SEQ ID NO: 8) and LacZ Reverse Primer: GTG TAG ATG GGC GCA TCG TAA C (SEQ ID NO: 9). Alternatively, the genotyping services of Transnetyx (Cordova, Tenn.) were used. Both Rik^{+/-} and Rik^{-/-} mice reach adulthood without gross morphologic abnormalities.

### Generation of 2510002D24Rik-HA Knock-In Mice.

The following oligonucleotides were used for cloning sgRNA T7 expression vector (System Biosciences, Palo Alto, Calif.): 5′-AGG GCC TCC CTC TAC CAC AGG AGA-3′ (SEQ ID NO: 10) and 5′-AAA CTC TCC TGT GGT AGA GGG AGG-3′ (SEQ ID NO: 11). In vitro transcription was performed using the MEGAshortscript T7 Kit (Thermo Fisher, Waltham, Mass.), and transcribed RNA was purified using MEGAclear RNA Kit (Thermo Fisher). The following single-stranded oligonucleotide was ordered as an Ultramer DNA oligonucleotide from Integrated DNA Technologies (Coralville, Iowa) and used as the DNA donor 5′-TCC AGG CTG CTC AGA AGC ACA CCT TGG TAT GGG CCT TGA GGC AAA GGC CCC CTA CGG ACT GGA ACC TCC CTC TAC CAC AGG AGA AAG ACA AGT ACC CAT ACG ATG TTC CAG ATT ACG CTT GAT AAG CAA CTG CAA CCC TAC CCT TGA CCT AGA CTT TGA CTG GCT CTT ACT TGA CCT GGA ACA CAG AGC AAA GCA TTT CC-3′ (SEQ ID NO: 12).

A mixture of 50 ng/μL sgRNA, 100 ng/μL hCas9 (System Biosciences), and 100 ng/μL single-stranded DNA donor was microinjected into the cytoplasm of C57BL6/J zygotes. The injected zygotes were transferred into pseudo-pregnant CD-1 female mice. The F₀ mice were genotyped by PCR using primers Rik-HA F: 5′-CTC TGT GAG AGC GAG CAG GTT CG-3′ (SEQ ID NO: 13) and Rik-HA R: 5′-TCC AGT GGG CAG CAG GCA AT-3′ (SEQ ID NO: 14). PCR products were purified, restriction digested with Hpy188III, and then analyzed by agarose gel electrophoresis. The sequences of PCR products, with correct sizes of bands, were further confirmed by subcloning and Sanger sequencing. F₀ mice with the correct mutation site were crossed with C57BL/6J mice. The genotype of F₁ mice was confirmed by PCR-restriction-digestion analysis and Sanger sequencing.

### Generation of Atp23 Knock-Out Mice

The following oligonucleotides were used for cloning sgRNA1 and sgRNA2 T7 expression vectors (System Biosciences):

sgRNA1F-
(SEQ ID NO: 15)
5′-AGG GCC AGT AAG AGA GAA GAA CGG-3′
sgRNA1R
(SEQ ID NO: 16)
5′-AAA CCC GTT CTT CTC TCT TAC TGG-3′;
and
sgRNA2F-
(SEQ ID NO: 17)
5′-AGG GTC ATA G CG GAT GTG GGA CAG-3′,
sgRNA2R-
(SEQ ID NO: 18)
5′-AAA OCT GTC CCA CAT CCG CTA TGA-3′.

In vitro transcription was performed using the MEGAshortscript T7 Kit and transcribed RNA was purified using MEGAclear RNA Kit. The following single-stranded oligonucleotide was ordered as an Ultramer DNA oligonucleotide from Integrated DNA Technologies and used as the DNA donor 5′-CCA TCT TTT TAG CTC CCT TCC TCC CTC CCT CCT TTC CTC TCC CTC CTC AGA GGT GAG AAC TGA ACC TAA GAC CTT GTG CTT ACC AGG CAA GGC CCT ACC ATG GCT GGT GAC GCT GTT CTG CGG TTT CAT GCA CGT TCT CCC GGC TGC TGC TTT GCT GAT GTT CTC TTC TGC CCT CAA GCG TTG AAG TCC CAC AGA CCT TT-3′ (SEQ ID NO: 19).

A mixture of 50 ng/μL sgRNAs, 100 ng/μL hCas9 (System Biosciences) and 100 ng/μL single-stranded DNA donors was microinjected into the cytoplasm of C57BL6/J zygotes. The injected zygotes were transferred into pseudo-pregnant CD-1 female mice. The F₀ mice were genotyped by PCR using primers:

Atp23 KO-Forward:
(SEQ ID NO: 20)
5′-TCCTCCCTCCCTCCTTTCCTCTC-3′;
Atp23 KO-Reverse:
(SEQ ID NO: 21)
5′-CCTCGGCAGAACACAGAAGAAGAAA-3′;
and
Atp23 wt-Reverse:
(SEQ ID NO: 22)
5′-AATGAGGGTTAGCAAGCAGAAATGTGT-3′.

PCR products were analyzed by agarose gel electrophoresis. The sequences of PCR products with correct sizes of bands were further confirmed by subcloning and Sanger sequencing. F₀ mice with the correct mutation site were crossed with C57BL6/J mice. The genotype of F₁ mice was confirmed by PCR analysis and Sanger sequencing as described above.

Generation of Mice with Conditional Deletion of 2510002D24Rik

Mice with the floxed allele of 2510002D24Rik (Rik^{+/f} mice) were designed by Ingenious Targeting Laboratory (Ronkonkoma, N.Y.). A 14-kb region used to construct the targeting vector was first subcloned from a positively identified C57BL/6 BAC clone (RP23-127H5) by using a homologous recombination-based approach. The region was designed such that the homologous long arm (LA) extends about 7.2 kb 5′ to the distal LoxP site. The LoxP cassette was inserted 623 bp upstream of exon 1. A LoxP-FRT-flanked Neo cassette was positioned 214 bp downstream of exon 3. The targeted region was 4.42 kb, including exons 1-3. The homologous short arm (SA) extended ˜2.4 kb downstream of the Neo cassette.

The final targeting vector was constructed using conventional cloning and recombineering methods. The targeting vector was confirmed by restriction analysis after each modification step and by sequencing using primers designed to read from the Neo-selection cassette into the 3′ end of the middle arm of the floxed/targeted region (iNeoN2) and from Neo to 5′ of the SA (iNeoN3). The single LoxP site was confirmed by sequencing with primer LOX1. Primers T73 and P6 anneal to the vector sequence and read into the 3′ and 5′ ends, respectively, of the homology arms.

The targeting vector (10 μg) was linearized and then transfected by electroporation of HF4 (129/SvEv×C57Bl/6J) (FLP Hybrid) ES cells. After selection with G418 antibiotic, surviving clones were expanded for PCR analysis to identify recombinant ES clones. The Neo cassette in the targeting vector was removed during ES clone expansion. Screening primer A2 was designed downstream of the SA, outside the 3′ region used to generate the targeting construct. PCR reactions using A2 with FRTN1C primer amplify the 2.69-kb fragment. Five clones were identified as positive, expanded, and reconfirmed for SA integration. PCR was performed on the 5 clones to detect the presence of the distal LoxP site by using the LOX2 and SDL3 primers. This reaction amplifies a 214-bp WT product. The presence of a second PCR product (58 bp larger than the WT product) indicates a positive LoxP PCR. Sequencing was performed on purified PCR DNA by using the LOX2 primer. The presence of the LoxP site was confirmed by DNA sequencing.

Secondary confirmation of positive clones identified by PCR was performed by Southern blot analysis. To confirm 5′ arm integration, DNA was digested with SpeI and electrophoretically separated on a 0.8% agarose gel. After transfer to a nylon membrane, the digested DNA was hybridized with an MPB3/4 probe targeted against the target region in the middle arm. DNA from HF4 mouse ES cells was used as a WT control. Positive clones were analyzed by Southern blot analysis for 3′-arm integration. DNA was digested with NcoI and electrophoretically separated on a 0.8% agarose gel. After transfer to a nylon membrane, the digested DNA was hybridized with probe MPB3/4. Two ES clones were confirmed by Southern blotting and were injected into 71 C57BL/6 blastocyst stage embryos with 8-12 cells for each clone. Embryos were transferred to uterus horns of pseudo pregnant CD-1 foster mice mated with vasectomized B6CBAF1/J studs and developed to term. Of the 19 pups born, 14 developed to mostly high male chimeras which were bred to germ line transmission.

Genotyping was performed either by PCR using LOX2 and SDL3 primers (WT allele, 214 bp; LoxP allele, 272 bp) or using Transnetyx genotyping services.

### Primers Used for Sequencing:

Primer P6:
(SEQ ID NO: 23)
5′-GAG TGC ACC ATA TGG ACA TAT TGT C-3′
Primer T73:
(SEQ ID NO: 24)
5′-TAA TGC AGG TTA ACC TGG CTT ATC G-3′
Primer LOX1:
(SEQ ID NO. 25)
5′-CTT GGT CAG GCT GGA AAG AG-3′
Primer iNeoN2:
(SEQ ID NO. 26)
5′-AGT ATG GCT TTC CTT CCC GAT GG-3′
Primer iNeoN3:
(SEQ ID NO: 27)
5′-TCT AAG GCC GAG TCT TAT GAG CAG-3′

### Primers for PCR Screening:

A2:
(SEQ ID NO: 28)
5′-TCC TAG CCA AAT GGA TGG AC-3′
FRTN1C:
(SEQ ID NO: 29)
5′-TCG TTC GAA CAT AAC TTC GTA TAG C-3′
LOX2:
(SEQ ID NO: 30)
5′-CCA GAT GAT CTA AGT ATA TGT GTT GCA C-3′
SDL3:
(SEQ ID NO: 31)
5′-CCT AAC TGG AGA TCA TAA GGT GAG ATG-3′

### MPB3/4 Probe Primers:

MPB3:
(SEQ ID NO: 32)
5′-CTT CTT TCA CCC TTA GTC ATC CT-3′
MPB4:
(SEQ ID NO: 33)
5′-GCA GTT TGG TAC TCA GGA GAG A-3′

### Mouse Behavior

Adult (16-26 weeks) male mice were used for all behavioral experiments. The experimenter was blind to genotypes of the mice. For the one-chamber social behavior tests, the interaction time was scored by an observer blind to genotypes for all experiments, except the one involving AAV-hDlx-Cre-GFP-injected mice, which was scored by Cleversys SocialScan software (Restin, Va.). The three-chamber social behavior test was also scored by Cleversys software. All other behavior tests were scored by Cleversys Topscan software (Restin, Va.).

One-chamber direct interaction test for social memory. This method was adapted.¹ Mice were housed in a holding room in 12 hour 12 hour light-dark cycle. Mice were single-housed for one week before habituating to a test arena (i.e., a mouse was allowed to navigate an empty arena for 10 minutes and then was returned to the home cage). One day later, the mouse was returned for the sociability test (trial 1). During the sociability test, the mouse was allowed to interact with a novel juvenile (3-4 weeks) male C57Bl/6J mouse for 5 minutes. The duration of active social interaction between the adult subject mouse and the juvenile stimulus mouse (anogenital and nose-to-nose sniffing, following, and allogrooming initiated by the test subject) was scored by an experimenter blind to the genotypes. After a one hour interval, the test was run again (trial 2) with either the previously encountered mouse or a novel juvenile (3-4 weeks) male C57Bl/6J mouse. A decrease in social interaction time spent in trial 2 with the previously encountered stimulus mouse compared to that in trial 1 was used as a measure of social (recognition) memory. Time spent interacting with the novel stimulus mouse during trial 2 was used as a measure of sociability. The duration of interaction was monitored via CleverSys video-acquisition software. The interaction time during 3-5 minutes of the trial was recorded. Mice that interacted for less than 30 seconds during familiarization (first novel mouse) were excluded from analysis.

A three-chamber social sociability and social novelty test was performed as follows. This method was adapted.³⁷ Experiments were performed in a three-chambered apparatus with three evenly spaced compartments (23.6″×15.5″×9.1). During the sociability test, mice were given a choice between investigating an empty holding chamber or a holding chamber that contains a novel con-specific adult mouse for 10 minutes. A stimulus mouse was placed in the left or right compartments (systematically alternated). The subject mouse was placed in the center compartment. After a one hour interval, the subject mouse was tested in a social novelty task for 10 minutes. In the social novelty task, the mice were given a choice between investigating a new, unfamiliar adult mouse or the previously investigated (familiar) mouse. The durations of interaction between the conspecific or unfamiliar mouse were monitored via CleverSys video-acquisition software.

An open-field test was performed as follows. Mice were allowed to navigate an open field (16″×16″) arena for one hour. The time spent in the enclosed corners, along the sides, and the center of the arena were assessed. Locomotor activity was monitored via CleverSys video-acquisition software and reported as the number of bouts, duration, and percentage of total time spent in the center and enclosed corners.

In testing grooming, the number of grooming bouts and duration and percentage of total time spent grooming were recorded in the open-field arena and analyzed using CleverSys software.

A rotarod test was performed as follows. Mice were placed on a rotating rod accelerating from 4 to 40 rpm in 4 minutes (Manufacturer). The time for the animal to fall off the rod (latency) and the distance covered before falling were reported.

A Morris water maze test was performed as follows. One hour prior to testing, animals were brought into the testing room and allowed to habituate. Testing was performed during the animal's inactive phase under dim-light conditions. Mice were allowed to navigate in the maze, with swimming patterns recorded using a video camera tracking system (HVS Image, Co., Buckingham, UK) mounted above the pool. Animals learned to find a hidden, clear platform by using the standard spatial version of the Morris water maze task for 4 successive days. Each day, animals were given four 1-minute trials from each starting position with an intertrial latency of one minute. The order of the starting locations was counterbalanced each day by using a Latin-square design. A spatial learning (probe) trial was administered one hour after the completion of spatial training. A spatial memory (probe) trial was administered 48 hours after completion of the spatial learning.

During both probe trials, the platform was removed, and the mice received a single 1-minute trial in which the animal tried to find the escape platform. These trials originated from the starting location that was the farthest from the platform's location throughout training. Mice also completed a nonspatial learning task at least seven days after completion of the spatial protocol. In that task, mice were trained to find a black visible platform for two successive days. During Day 1, the escape platform was located in the same position used during spatial training. The next day, the escape platform was moved to a new quadrant. Each day, the mouse was given four 1-minute trials in the same manner that occurred during spatial training. To avoid hypothermia, animals were dried with paper towels and placed in warmed holding cages immediately after each round of training and testing trials.

The novel object recognition test was performed as follows. The testing arena consisted of a vinyl, opaque cylinder approximately 40 cm in diameter with walls 40 cm tall. Before recognition memory was determined, the mouse was first habituated to an empty testing arena for 3 to 10 minutes. On the day of testing, the mice were habituated to the testing arena for 3 to 10 minutes, and then returned to their home cages for 5 to 10 minutes. Next the mice were placed back in the testing arena for 3 to 15 minutes with two to four identical objects (T1). Objects used are custom-fabricated plastic with an overall size of approximately 4 cm in height×4 cm in diameter. The mice were then placed back into their home cages. One to 48 hours later, each mouse was returned to the testing arena for 3 to 10 minutes. The mice were then exposed to one to three identical objects and one novel object (T2). The activity of the mice was video recorded and scored using visual-tracking software (CleverSys Inc.) The amount of time the animals explored the novel object relative to the familiar object was reported.

Fear conditioning testing was performed as follows. Mice were individually placed in a conditioning chamber with the room light on and allowed to explore the testing chamber for 2 minutes before a discrete conditioning stimulus (CS) was delivered in the form of a 30 second tone [10 kHz, 75-dB sound pressure level (SPL)]. Within the last 2 seconds of the tone, an unconditioned stimulus (US) was delivered in the form of a mild foot shock (0.5 mA, 2 s). Mice were allowed to recover for one minute, and then three more CS-US pairs were delivered. After the last CS-US pairing, mice remained in the conditioning chamber for one minute and were then returned to the home cage.

Approximately 24 hours later, mice were placed in a new environment with the light off and allowed to explore for two minutes, followed by exposure to only the CS tone for 30 seconds. After a recovery period of 30 seconds, the tone exposure and recovery period steps were repeated three times. The percentage of freezing times during the training period and during the pre-CS and post-CS periods on the test day were compared across groups using Video Freeze software (Med Associates). For contextual-fear conditioning, mice were placed in the same environment as the conditioning day with lights on. The percentage of freezing relative to the total five minute recording was reported.

An Elevated plus maze test was performed as follows. Mice were tested individually in the elevated plus maze. The testing apparatus stood 50 cm above the floor with two open arms and two closed arms of equal dimensions (50 cm×12 cm) extending away from a square central platform. Plexiglas lateral walls that are perpendicular to the two open arms enclosed the two closed arms. A plexiglass rim (1-cm high) designed to reduce the possibility of falls surrounded the open arms. Mice were released facing an open arm and tested for 5 minutes. The amount of time spent in each of the center, open, and closed arms was monitored via CleverSys video-acquisition software.

Olfactory habituation testing was performed as previously described.³⁸ To begin the experiment, mice were individually placed in a clean mouse cage and allowed to habituate for one hour. Next, the mouse was habituated to the testing chamber (clear plexiglass chamber measuring 40 cm×40 cm×35 cm) for 10 minutes. During the habituation period, a clean cotton swab was presented in 1 of the corners of the testing arena. For the test phase, five odors (water, 2 foreign neutral odors (almond, mint) and 2 social scents (male non-con-specifics and female non-con-specifics)) were presented three times each for two minutes. The order in which these odors were presented is as follows: 3× water, 3× almond, 3× mint, 3× male, 3× female. After each two minute presentation, the mouse was returned to a clean holding cage for one minute. To prepare test sessions, a 1:100 dilution of the pure almond and mint extracts (McCormick) solutions in H₂O were prepared each day. Cotton applicators for social scents were made by swapping the bottom of dirty mouse cages containing either male or female mice. The sniffing duration of each mouse at the cotton swab was analyzed with CleverSys TopScan Software.

### Whole-Cell Electrophysiology

Mouse brains were quickly removed and placed in cold (4° C.) dissecting solution containing 125 mM choline-CI, 2.5 mM KCl, 0.4 mM CaCl₂, 6 mM MgCl₂, 1.25 mM NaH₂PO₄, 26 mM NaHCO₃, and 20 mM glucose (295-300 mOsm), under 95% O₂/5% CO₂. Acute transverse hippocampal slices (400-μm thick) were prepared. After dissection, slices were incubated for 45 minutes in artificial cerebrospinal fluid (aCSF) containing 124 mM NaCl, 2.5 mM KCl, 2 mM CaCl₂, 2 mM MgCl₂, 1.25 mM NaH₂PO₄, 26 mM NaHCO₃, and 10 mM glucose (285-295 mOsm), under 95% O₂/5% CO₂ at 32° C. to 34° C. and then transferred into the submerged recording chamber and superfused (1.5-2 mL/min) with aCSF. Electrophysiology and two-photon imaging experiments were conducted in a perfusion chamber maintained at 32-33° C. Electrophysiological data were acquired using a Multicamp 700B amplifier, filtered at 2 kHz and digitized at 10/20 kHz using a Digidata 1440 digitizer controlled by Clampex acquisition software.

All offline analyses of electrophysiological data was performed in Clampfit. Input-output relations of evoked compounds (EPSP/IPSP) in CA2 neurons were measured by stimulating the Schaffer collaterals or the entorhinal cortex inputs with a concentric bipolar stimulating electrode (125-μm outer diameter; 12.5-μm inner diameter) connected to an Iso-Flex stimulus isolator with 100-μs pulses. Stimulating and recording electrodes were separated by at least 350 to 400 μm. The recording electrodes were borosilicate glass capillaries pulled in a Sutter P-1000 puller. The recording electrodes had a resistance of approximately 3.5-5 mΩ. The CA2 pyramidal neurons were recorded in whole-cell current-clamp mode using electrodes filled with 130 mM K gluconate, 10 mM KCl, 0.1 mM EGTA, 2 mM MgCl₂, 5 mM NaCl, 2 mM ATP-Na₂, 0.4 mM GTP-Na, 10 mM HEPES, and 10-25 μM Alexa 594 (pH 7.35, ˜290 mOsm). EPSPs were recorded by holding the cells at −80 mV (accounting for a liquid junction potential of ˜14 mV). AP firing was recorded directly from fast-spiking interneurons, which were identified based on their smaller size compared to pyramidal neurons and electrophysiological properties, such as higher membrane resistance (>150 mΩ) and lower capacitance (<100 pF).

IPSCs were measured in whole-cell voltage-clamp mode in the cells at 0 mV (accounting for a liquid junction potential of ˜11 mV) using a Cesium-based internal solution of 125 mM CsMeSO₃, 10 HEPES mM, 0.1 mM EGTA, 2 mM CsCl, 5 mM QX314, 5 mM TEA-CI, 10 mM Na₂ creatine phosphate, 4 mM MgATP, 0.3 mM NaGTP. Access resistance was monitored using a −5 mV step after each recording (range, 25-50 mΩ), with cells that displayed unstable access resistance (variations >20%) excluded from the analyses. GABA_{A}R antagonist SR95531 (1 μM) and GABA_{B}R antagonist CGP55845 (1 μM) were added to the perfusion aCSF in some experiments, as indicated in the figures, to block inhibition. D-AP5 (100 μM) and DNQX (10 μM) were added to the perfusion aCSF to measure iLTD. Synaptic plasticity (iLTD and CA2 disinhibition) experiments were performed as previously described.⁸ High-frequency stimulation (100 pulses at 100 Hz repeated twice) was applied after baseline recordings. The magnitude of plasticity was measured by comparing averaged responses at 30 to 40 minutes for iLTD and 35 to 40 minutes for CA2 disinhibition after induction, with baseline-averaged responses 10 minutes before induction.

### Two-Photon Imaging of PercevalHR

These experiments were performed in acute hippocampal slices (see above). Two-photon laser-scanning microscopy was performed using an Ultima imaging system (Prairie Technologies, Middleton, Wis.), a Ti:sapphire Chameleon Ultra femtosecond-pulsed laser (Coherent Inc., Santa Clara, Calif.), and 60× (0.9 NA) water-immersion IR objectives (Olympus, Center Valley, Pa.). Changes in fluorescence of PercevalHR were measured by 12-second line scans spanning a segment of cell body and a proximal part of a dendrite. A 300-pA current step was applied for 2 seconds to evoke a train of APs. PercevalHR was excited at 940 nm and 840 nm for measuring changes in ATP and ADP, respectively. Fluorescence data from 1-3 scans repeated at 2- to 3-min intervals were averaged. Data were imported into Clampfit for further processing and corrected for bleaching using the difference between the beginning and the end of the 12-s scan. The line-scan traces were filtered at 23 kHz and box-car averaged. ΔF/F₀ was calculated using a 400-ms baseline (F₀) measured before the onset of the current step.

### Subcellular Fractionation of Synaptosomes and Nonsynaptic Mitochondria

Mitochondrial and synaptosomal fractions were prepared using a Percoll gradient centrifugation method adapted from previous protocols.^{39,40} Mice were decapitated, and brains were quickly removed and placed in cold (4° C.) dissection buffer containing 125 mM choline-CI, 2.5 mM KCl, 0.4 mM CaCl₂, 6 mM MgCl₂, 1.25 mM NaH₂PO₄, 26 mM NaHCO₃, and 20 mM glucose (295-300 mOsm), under 95% O₂/5% CO₂. Hippocampi from both hemispheres were isolated in the cold (4° C.) dissection buffer and homogenized in a Dounce homogenizer with 500 μL mitochondrial isolation buffer (containing 225 mM sucrose, 75 mM mannitol, 5 mM HEPES, and 1 mM EGTA). Equal volumes of 24% Percoll solution and the isolation buffer were added to the homogenate to prepare a sample containing 12% Percoll. This sample was layered on a gradient of 26% and 40% Percoll (in isolation buffer) in ultra-clear centrifuge tubes and centrifuged at 30,700×g for 10 minutes with slow acceleration and deceleration (no brakes). The myelin layer accumulated at the top was discarded. The next layer containing synaptosomes and the layer between 26% and 40% Percoll containing mitochondria were aspirated into separate tubes. The separated fractions were further diluted with isolation buffer and centrifuged twice at 16,700×g for 10 minutes to form firm pellets. Protease inhibitor cocktail [cOmplete™ Mini Protease Inhibitor Cocktail, Sigma-Aldrich (St. Louis, Mo.); 1 tablet diluted in 10 mL] was added to the pellet and stored at −80° C. for Western blot analysis. For proteomics, fractionated samples were extracted in a urea lysis buffer and stored at −80° C.

### Proteomics

Protein digestion and peptide isobaric labeling by tandem mass tags were performed. The experiment was performed with a previously published protocol^{41,42} with slight modification. Whole hippocampal tissue or synaptosomal and mitochondrial fractions, prepared as described above, were extracted in lysis buffer (50 mM HEPES, pH 8.5, 8 M urea, and 0.5% sodium deoxycholate). The protein concentration of the lysates was determined by a Coomassie-stained short gel with bovine serum albumin (BSA) as a standard. Protein (100 μg) for each sample was digested with LysC (Wako Chemicals, Richmond, Va.) at an enzyme-to-substrate ratio of 1:100 (w/w) for 2 hours in the presence of 1 mM DTT. Following this, the samples were diluted to a final 2 M urea concentration with 50 mM HEPES (pH 8.5) and further digested with trypsin (Promega, Madison, Wis.) at an enzyme-to-substrate ratio of 1:50 (w/w) for at least 3 hours.

The peptides were reduced by adding 1 mM DTT for 30 minutes at room temperature (RT) followed by alkylation with 10 mM iodoacetamide (IAA) for 30 minutes in the dark at RT. The unreacted IAA was quenched with 30 mM DTT for 30 min. Finally, the digestion was terminated and acidified by adding trifluoroacetic acid (TFA) to 1%, desalted using C18 cartridges (Harvard Apparatus, Cambridge, Mass.), and dried by speed vac. The purified peptides were resuspended in 50 mM HEPES (pH 8.5), labeled with 10-plex TMT reagents (Thermo Fisher) following the manufacturer's recommendation.

Two-dimensional HPLC and mass spectrometry were performed as follows. TMT-labeled samples were mixed equally, desalted, and fractionated on an offline HPLC (Agilent 1220) using basic pH reverse-phase liquid chromatography (pH 8.0, XBridge C18 column, 4.6 mm×25 cm, 3.5 μm particle size; Waters, Milford, Mass.). The fractions were dried, resuspended in 5% formic acid, and analyzed by acidic pH reverse-phase LC-MS/MS analysis. The peptide samples were loaded on a nanoscale capillary reverse-phase C18 column [new objective, 75 μm ID×˜40 cm, 1.9 μm C18 resin (Dr. Maisch GmbH, Ammerbuck-Entringen Germany)] by an HPLC system (Waters nanoAcquity), and eluted by a 180-min gradient. The eluted peptides were ionized by electrospray ionization and detected by an inline Orbitrap Fusion mass spectrometer (Thermo Fisher). The mass spectrometer was operated in data-dependent mode with a survey scan in Orbitrap (60,000 resolution, 2×10⁵ AGC target and 50-ms maximal ion time) and MS/MS high-resolution scans (60,000 resolution, 1×10⁵ AGC target, 150-ms maximal ion time, 38 HCD normalized collision energy, 1 m/z isolation window, and 20-s dynamic exclusion).

Mass spectrometric data analysis was performed as follows. The MS/MS raw files were processed by a newly developed tag-based hybrid search engine JUMP, which showed better sensitivity and specificity than commercial packages (e.g., Proteome Discoverer).³ The data were searched against the UniProt mouse database, concatenated with a reversed-decoy database for evaluating false-discovery rates. Searches were performed using 10 ppm mass tolerance for precursor ions and 15 ppm mass tolerance for fragment ions, fully tryptic restriction with two maximal missed cleavages, three maximal modification sites, and the assignment of a, b, and y ions. TMT tags on lysine residues and N termini (+229.162932 Da) were used for static modifications, with methionine oxidation (+15.99492 Da) considered a dynamic modification. MS/MS spectra were filtered by mass accuracy and matching scores to reduce the protein false-discovery rate to ˜1%. Proteins were quantified by summing reporter ion counts across all matched peptide-to-spectrum matches using the JUMP software suite.

### Immunoprecipitation Assay

Rik^{HA} or WT mouse brains were used for immunoprecipitation assays. Brains were resuspended with RIPA buffer containing protease inhibitors and sonicated twice at 15% amplitude for 10 seconds in the sonifier (Branson, Danbury, Conn.) on ice. The lysate was then cleared by centrifugation at 13,000×g for 10 minutes at 4° C. The supernatant was first precleared by incubating with 30 μL protein-G agarose beads (Santa Cruz Biotechnology, Dallas, Tex.) for one hour at 4° C. with rotation. Meanwhile, 40 μL of 50% slurry protein-G agarose beads (Santa Cruz Biotechnology) were equilibrated with RIPA buffer and incubated with 2 μg anti-HA antibody (ab18181, Abcam, Cambridge, Mass.) at RT for one hour. After excess antibody was washed from the beads, precleared brain lysate was added and incubated overnight at 4° C. The beads were collected by centrifugation and washed three times with RIPA buffer. Bound proteins were mixed with 2× sample buffer containing 10% (v/v) 2-mercaptoethanol, and analyzed by performing immunoblotting with specific antibodies as indicated below.

### Western Blotting

The protein sample was fractionated by using SDS-polyacrylamide gel electrophoresis and transferred onto a PVDF membrane (Thermo Fisher). After incubation with 5% (wt/vol) nonfat dry milk in TBST (10 mM Tris. pH 8.0, 150 mM NaCl, and 0.5% (vol/vol) Tween 20) for 30 minutes, membranes were incubated with antibodies against HA (1:1000 dilution; ab18181, Abcam), ATP23 (1:500 dilution; NBP1-81339, Novus; St. Charles, Mo.), or actin (1:5000 dilution; Sigma-Aldrich) at RT for one hour. Membranes were washed three times for 5 minutes each, and incubated with a 1:3000 dilution of horseradish peroxidase-conjugated anti-mouse or anti-rabbit antibodies (Santa Cruz Biotechnology) at RT for one hour. Blots were washed with TBST three times, and developed by using the ECL system (Pierce Biotechnology Inc., Rockford, Ill.).

### Quantitative Real-Time PCR

RNA was isolated from the hippocampus by using the mirVana RNA isolation kit (Ambion, Life Technologies, Foster City, Calif.). The iScript cDNA Synthesis Kit (Bio-Rad, 170-8891) was used to synthesize cDNA from 1 μg RNA. The following primers were used in the qPCR experiments:

2510002D24Rik:
(SEQ ID NO: 34)
5′-GTGTTCCAGGTCAAGTAA-3′
and
(SEQ ID NO: 35)
5′-AGAAGGACAAGTGATAAGC-3′,
U6:
(SEQ ID NO: 36)
5′-CGCTTCGGCAGCACATATAC-3′
and
(SEQ ID NO: 37)
5′-TTCACGAATTTGCGTGTCAT-3′.

RT-qPCR was performed using SYBR green in an Applied Biosystems 7900HT Fast Real-time PCR system with the standard protocol. A serial dilution of cDNA was used to generate a standard curve for each primer set, and this curve was used to calculate gene concentrations for each sample. All samples were run in duplicate.

### Immunohistochemistry and Confocal Imaging

Mice were anesthetized with ethyl carbamate (1.5 g/kg, 25% solution, intraperitoneal) and perfused transcardially with phosphate-buffered saline (PBS) for 1-2 minutes and then a fixative (4% paraformaldehyde in PBS). Brains were isolated and stored at 4° C. overnight in the same fixative. Sagittal sections (60-μm thick) were then prepared on a Leica vibratome. Sections with hippocampal regions were chosen for immunohistochemistry. Sections were permeabilized using 0.3% Triton-X PBS for 30 minutes followed by blocking in 0.3% BSA in PBS. Sections were then incubated in primary antibodies for about 24 hours at 4° C. and in secondary antibodies for four to six hours at RT. The secondary antibodies were washed away using PBS, and then Hoechst nuclear reagent (1:2000) was added for 30 min. Sections were washed using PBS 3-5 times between incubations and mounted on glass slides with 50% glycerol in PBS.

The primary antibodies used were: rabbit polyclonal anti-PV (1:2000, Swant Inc., catalog no. PV25), goat polyclonal anti-PV (1:200, SantaCruz Biotechnology, catalog no. SC7449), mouse anti-reelin (1:1000, R & D systems, catalog no. AF3820), rat monoclonal anti-somatostatin (1:200, Millipore, MAB354), rabbit polyclonal anti-ATP23 (XRCC6BP1; 1:200; Novus Biologicals, catalog no. NBP1-81339), mouse monoclonal anti-RGS14 (1:250; Neuromab, catalog no. 75-170), chicken anti-GFP (1:500; Abcam Inc.). Alexa-conjugated secondary antibodies (1:1000; 488, 555, 647) raised in donkey or goat against primary antibody species were obtained from ThermoFisher. Mounted sections were imaged in a Zeiss LSM780 confocal microscope with a pinhole setting of 1 airy disc. Z-stacks of tiled images were stitched using the Zen2.3 software.

For HA immunostaining, the sections were subjected to heat-mediated antigen retrieval in 0.01 M sodium citrate buffer (pH 6.0) containing 0.05% Tween 20 (Sigma) for 20 minutes at 98° C. and cooling for 30 minutes at room temperature. Endogenous peroxidase activity was inhibited by incubating the sections in 3% hydrogen peroxide/water for 10 minutes. After blocking with 10% normal goat serum (Vector Labs) in PBS, sections were incubated overnight at 4° C. with rat anti-HA (Roche, Cat #11867423001). HA immunoreactivity was detected using ImmPRESS® HRP anti-rat polymer detection kit [(Vector Labs, Cat #MP-7444), and Alexa Fluor™ 488 Tyramide Reagent (Thermo Fisher Scientific, Cat #B40953)]. For double labeling, the sections were then incubated with one of the following antibodies: goat anti-PV, rabbit anti-ATP23, mouse anti-RGS14, or anti-CYC. A respective Alexa-594-labeled secondary antibody (Thermo Fisher Scientific) was used to reveal the immunocomplexes.

### Generation of Plasmids and Viruses

AAV-hsyn-PercevalHR was generated as follows. To generate AAVs expressing syn-Perceval, PCR was used to amplify the human synapsin promoter (hSyn) from pAAV-6P-SEWB. The pAAV-GFP (Addgene, plasmid 32395) was cut with SnaB1 and Sac1 to replace CMV with hSyn (pAAV-hSyn-GFP). The PCR product of PercevalHR from plasmid GW1-PercevalHR (Addgene plasmid 49082) was cut with Sal1 and EcoR1, and then ligated into pAAV-hsyn-GFP.

pAAV-fsst-GFP-T2A-Atp23 and pAAV-hdIx-Cre-T2A-GFP was generated as follows. Coding sequences of the Atp23(NM_001159559.1) were amplified with the following primers: Atp23 F HindIII: 5-TAAAGCTTATGGCAGGAGCTCCGG-3′ (SEQ ID NO: 38) and ATP23 R HindIII: 5′-TAAAGCTTTCATATGTTGGAGTAGTAG-3′ (SEQ ID NO: 39) from cDNA generated from reverse-transcribed mouse whole-brain RNA using the Superscript First-Strand Synthesis RT-PCR kit (Invitrogen, Carlsbad, Calif.), subcloned into pAAV-CamKII-GFP-2A vector plasmid (from the St. Jude Vector Core) after cutting with HindIII. Then GFP-T2A-Atp23 was replaced with RFP of pAAV-fSST-RFP plasmid (Addgene, plasmid 22913) to create the pAAV-fSST-GFP-T2A-Atp23 plasmid. The pAAV-fsst-RFP was purchased from Addgene (Watertown, Mass.; plasmid 22913).

pAAV-hDlx-Cre-T2A-eGFP was generated as follows. The Cre-T2A-eGFP fragment was PCR-amplified from pAAV-CMV-Cre-T2A-eGFP by using 2 PCR primers, Cre F SaII: 5′-ATGTCGACCACCATGTCCAATTTACTGACC-3′ (SEQ ID NO: 40) and eGFP R AscI 5′-ATGGCGCGCCTTACTTGTAAAGCTCGTC-3′ (SEQ ID NO: 41). The fragment was then digested with SaII and AscI, and then inserted between the SaII and AscI sites of pAAV-hDlx-Flex-GFP-Fishell_6 (Addgene, plasmid 83895).

### Stereotaxic Injection of AAV Constructs

Mice were anaesthetized using isoflurane (2% for induction and 1.5% for maintenance) in 100% oxygen, with their heads restrained on a stereotaxic apparatus. An approximately 1-cm midline incision was made centered about 0.25 cm behind bregma. Viruses were injected into 2 locations within the CA2 region, in one or both hemispheres. The stereotaxic coordinates for the two injections in relation to the bregma were as follows: (1) −1.6 mm anteroposterior, 1.6 mm lateral, and 1.7 mm deep; (2) 2 mm anteroposterior, 2.6 mm lateral, and 1.8 mm deep. For behavioral experiments, injections were restricted to the anterior location in both hemispheres. Craniotomy holes were drilled at these locations, and 300 nL of AAVs was slowly (30 nL/min) injected via a 33G cannula. After each injection, the cannula was left in place for 2-3 minutes before being retracted. Following injections, the skin was sutured, and the mice were returned to the holding cages after recovery from anesthesia. Imaging and electrophysiology experiments were performed 4-7 weeks after AAV injections. During each experiment, care was taken to limit the differences in post-injection duration to a maximum of 5 days across experimental groups to avoid substantial differences in the levels of AAV expression.

### Electron Microscopy

Mice were anesthetized with ethyl carbamate (1.5 g/kg, 25% solution, intraperitoneal) and then perfused transcardially with PBS for 1-2 minutes and then with a fixative (2.5% glutaraldehyde and 2% paraformaldehyde in 0.2 M sodium cacodylate). Brains were isolated, stored at 4° C. overnight in the same fixative, and sagittal sections (100-μm thick) were prepared on a Leica vibratome. Smaller regions (˜500 μm×500 μm) containing the stratum radiatum of the CA2 hippocampal region were processed for 3D transmission electron microscopy. The samples were stained using a modified heavy metal-staining method, processed through a graded series of alcohol and propylene oxide, and then embedded in Epon hard resin.⁴⁴ Sections (0.5-μm thick) were cut to determine the correct area and then coated with iridium in a Denton Desk II sputter coater. The 3D electron microscopy images were collected on a Helios Nanolab 660 Dualbeam system. From the 3D stacks of electron micrographs (5×5×10 nm³ voxel size, 250-260 sections of 10-nm thickness and approximately 30×20 μm² area), synapses were identified based on the presence of postsynaptic densities and presynaptic vesicles. The 3D stacks were then analyzed using the cell-counter plugin in ImageJ. Representative movies of asymmetric and symmetric synapses containing mitochondria were generated using Amira 6.0 software.

### Other Drugs and Chemicals

SR95531, CGP55845, D-AP5, and DNQX were obtained from Tocris Bioscience (Minneapolis, Minn.). Stock solutions of these drugs were prepared in manufacturer-recommended solvents and stored at −20° C.

### Quantification and Statistical Analyses

Data are presented as mean±SEM in dot or bar graphs and median with 25^{th} to 75th percentile box plots and error bars denoting 10th and 90^{th} percentiles. Statistical analyses were performed using Sigmaplot 12.5 software. Statistical analyses were carried out using data from single cells for electrophysiological experiments and mice for behavioral and molecular experiments. Parametric or nonparametric tests were chosen based on the normality and variance of data distribution. Independent or paired t-tests, Mann-Whitney Rank-Sum test, One-Way ANOVA/Kruskal Wallis one-way ANOVA on ranks followed by a multiple-comparison procedure (Dunn's method), two-way ANOVA followed by Holm-Sidak multiple-comparison procedure, one/two-way RM ANOVA were the statistical tests used. Differences with P<0.05 were considered significant.

### Results

Silencing or lesioning the CA2 area of the murine hippocampus is known to cause a specific deficit in social-recognition memory, with no change in sociability or spatial and contextual memories typically associated with the hippocampus.^{1,5} Described herein are similar behavioral phenotypes present in mice carrying a deletion of the 2510002D24Rik gene (Rik^{+/-} and Rik^{-/-} mice), the expression of which is strongest in the CA3 and CA2 areas of the hippocampus.¹² Adult (>16 weeks) mice were used in the experiments. As described below, the expression of 2510002D24Rik was reduced about 50% in Rik^{+/-} mice, and was not detected in Rik^{-/-} mice (see FIG. 5B). Both mutants were deficient in social memory measured in the one-chamber direct-interaction and 3-chamber social novelty tests.^{1,15}

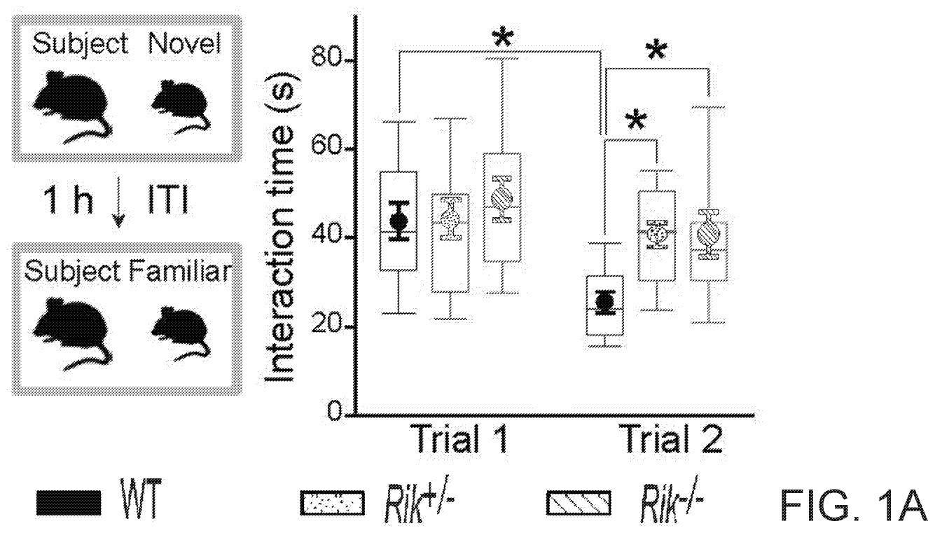

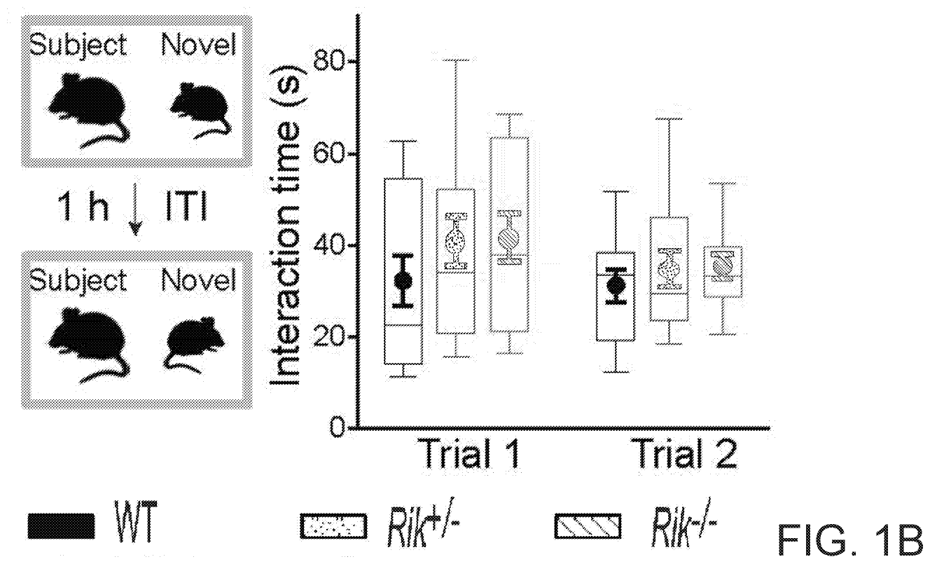

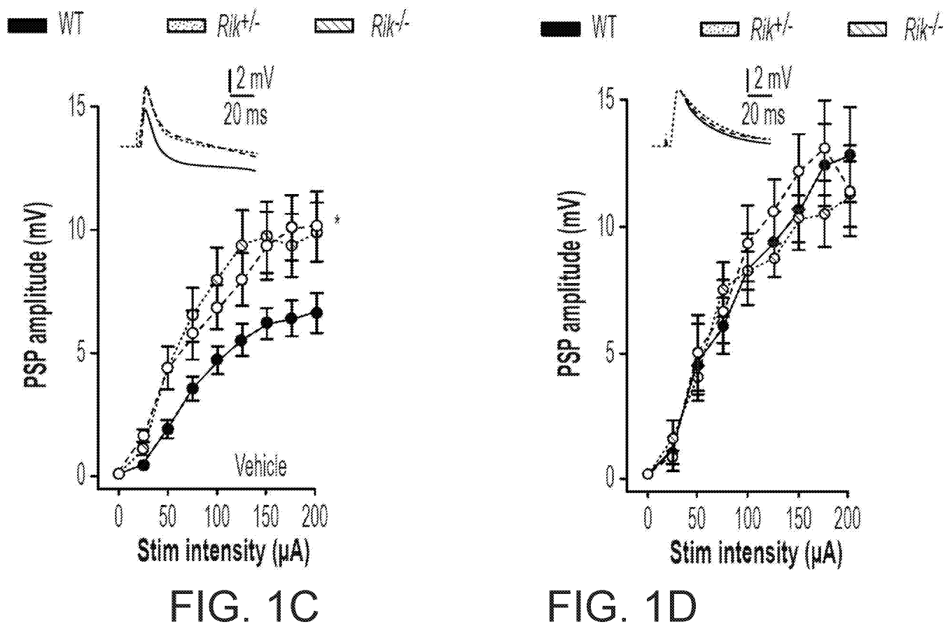

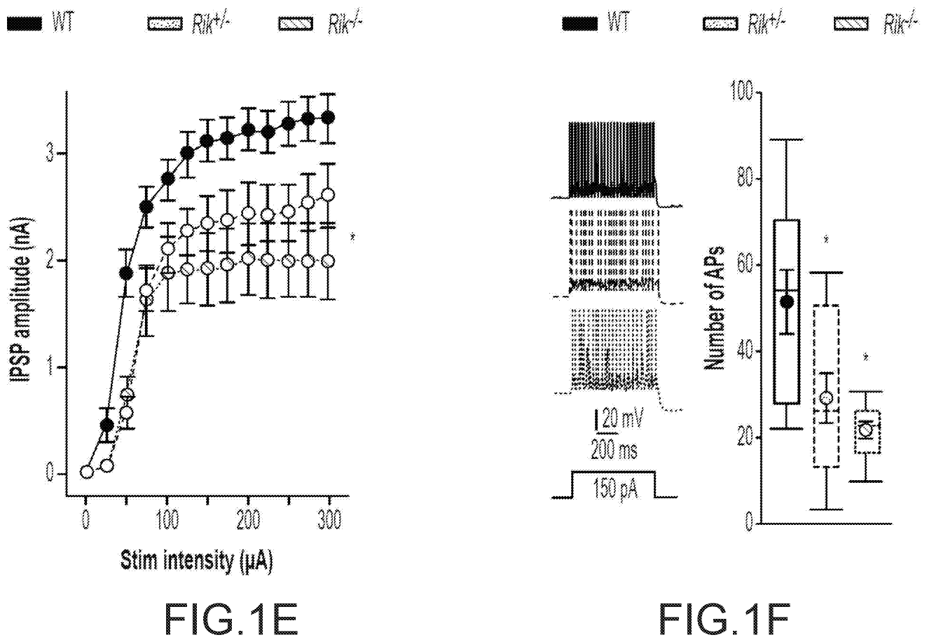

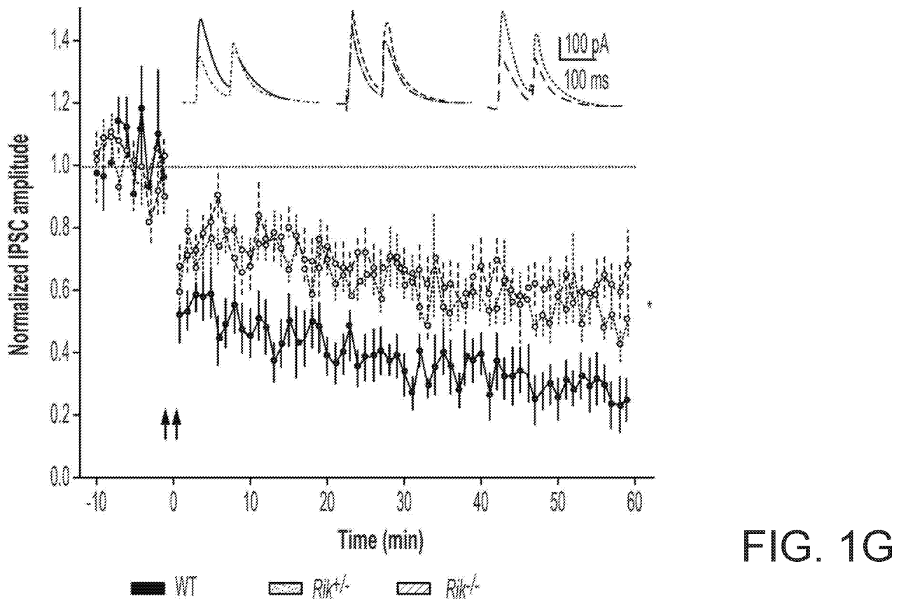

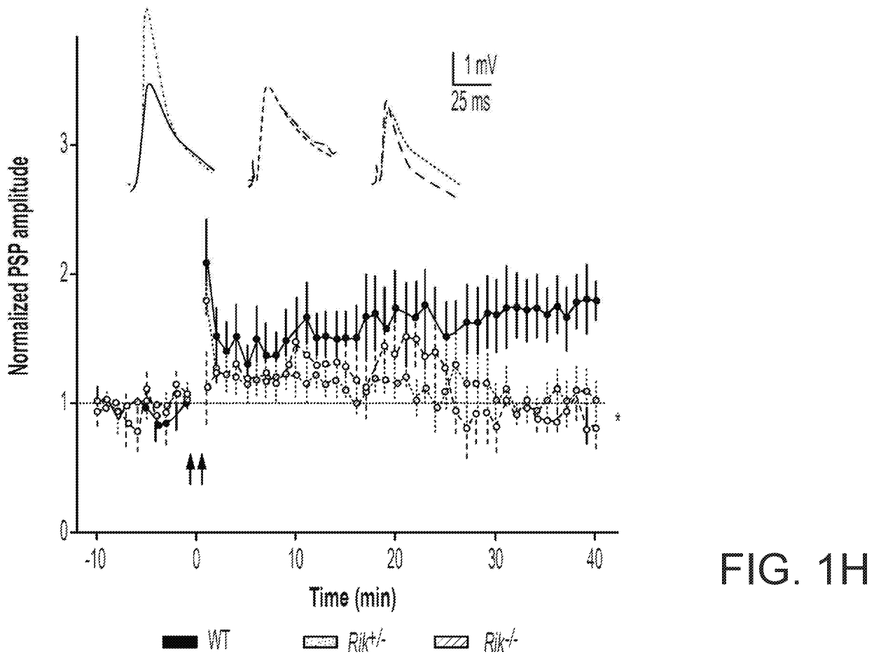

In the direct-interaction test, a subject mouse was exposed to an unfamiliar mouse in the first trial (trial 1). After a one hour intertrial interval, the subject mouse was either re-exposed to the same mouse (social recognition/memory test) or another unfamiliar mouse (sociability test) in trial 2. WT, Rik^{+/-}, and Rik^{-/-} mice were tested as subject mice. The investigation time for each subject mouse was tested. The data is shown in FIG. 1A for the same mouse, and in FIG. 1B for the unfamiliar mouse.

Social memory, measured as the decreased time a subject mouse spends exploring a previously encountered mouse, was reduced in both mutants compared to wild-type (WT) controls, as shown in FIG. 1A. In contrast, mutant mice were not significantly different from WT littermates in sociability, as shown in FIG. 1B. Rik^{+/-}, Rik^{-/-}, and WT subject mice showed similar, unchanging exploration durations during trials 1 and 2 when different novel mice were encountered in the 2 trials.

Social memory deficit was confirmed in the social novelty test in both mutants. In this test, social recognition was measured by the increased time that a subject spent interacting with a novel unrelated mouse, compared with that spent interacting with a familiar littermate (FIG. 5C, left). WT mice demonstrated a significant preference for the compartment containing the novel animal, whereas Rik^{-/-} and Rik^{-/-} mice did not (FIG. 5C, right). This deficit was not due to a decrease in sociability because the subject mice of all three genotypes showed a similar preference for a chamber containing a novel mouse versus an empty chamber (FIG. 5D).

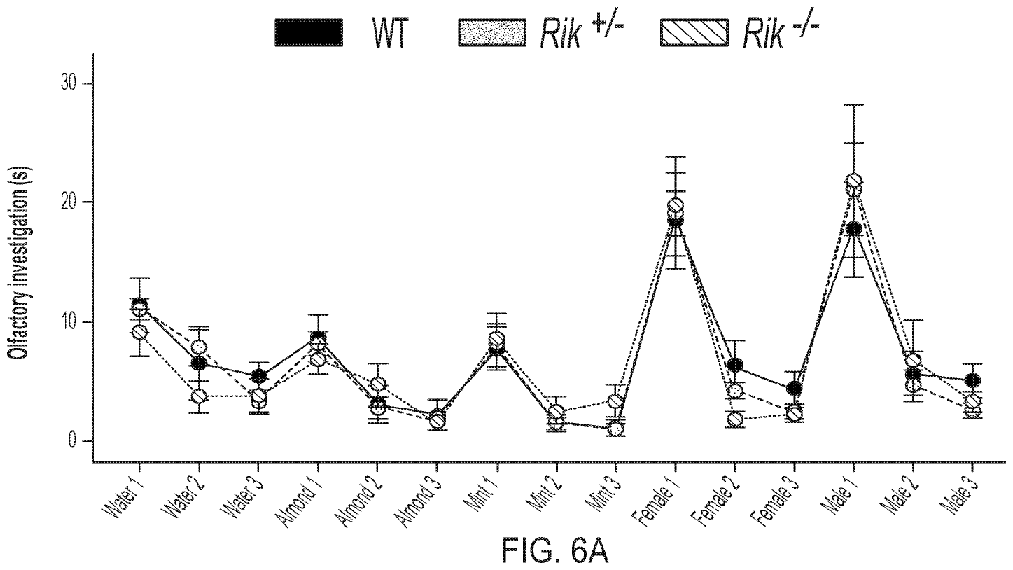

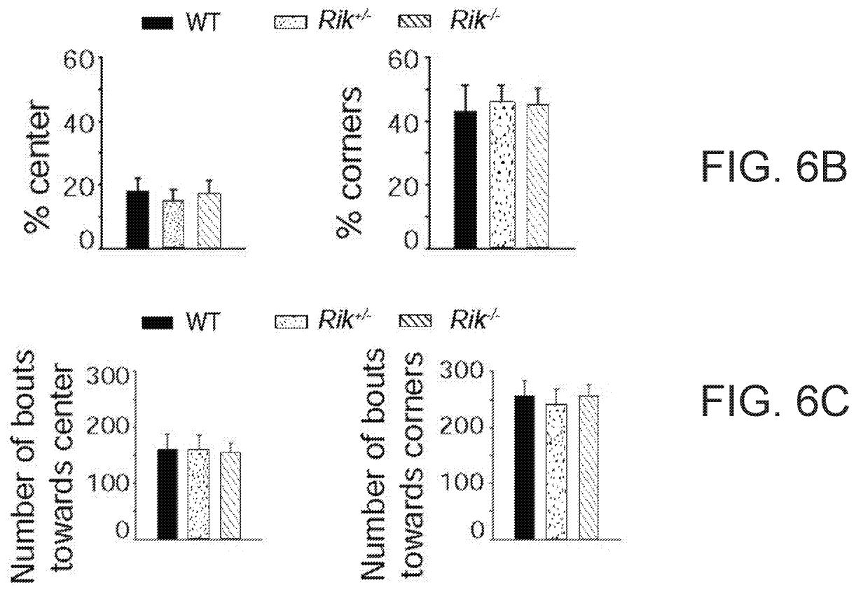

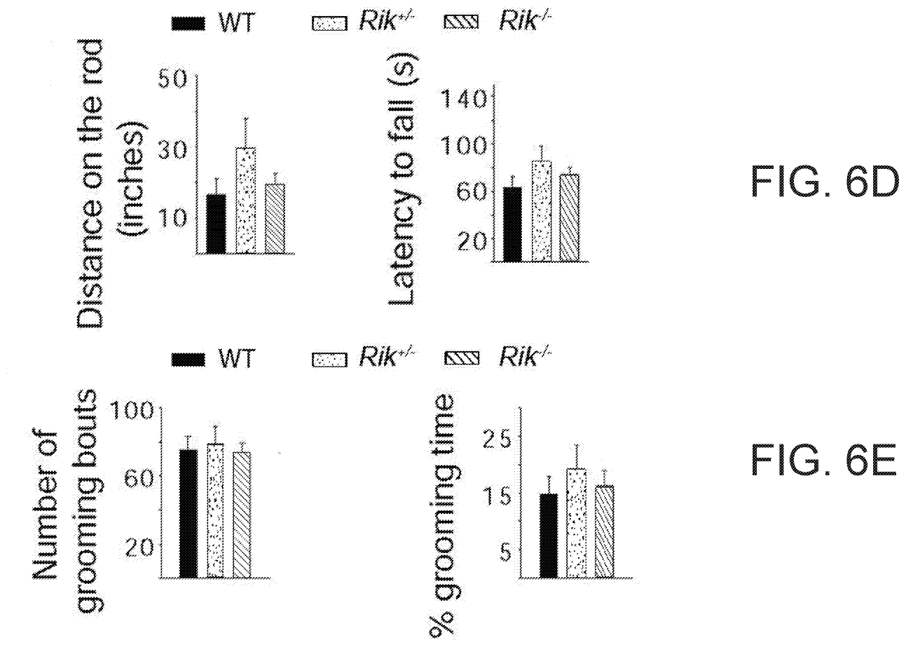

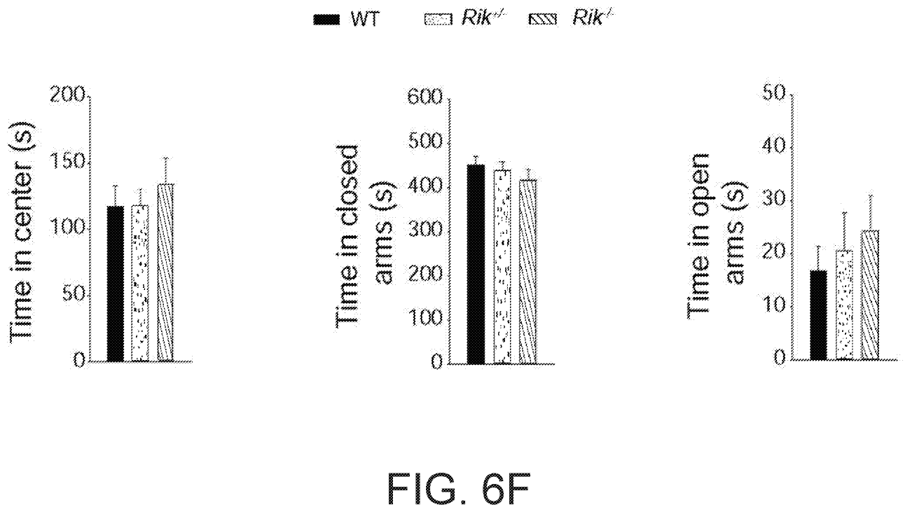

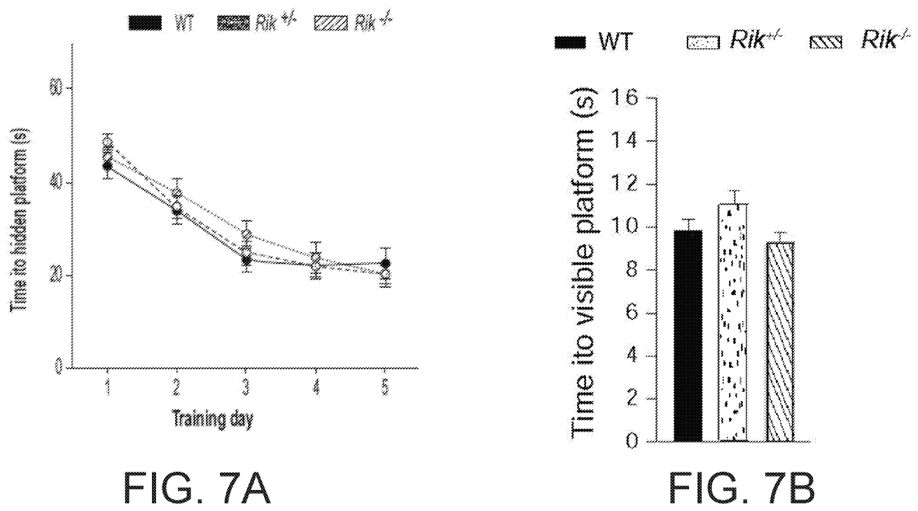

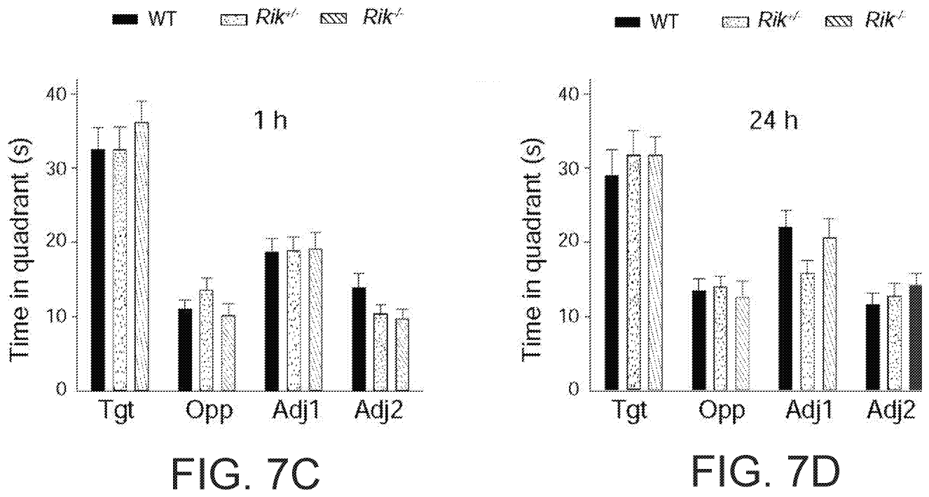

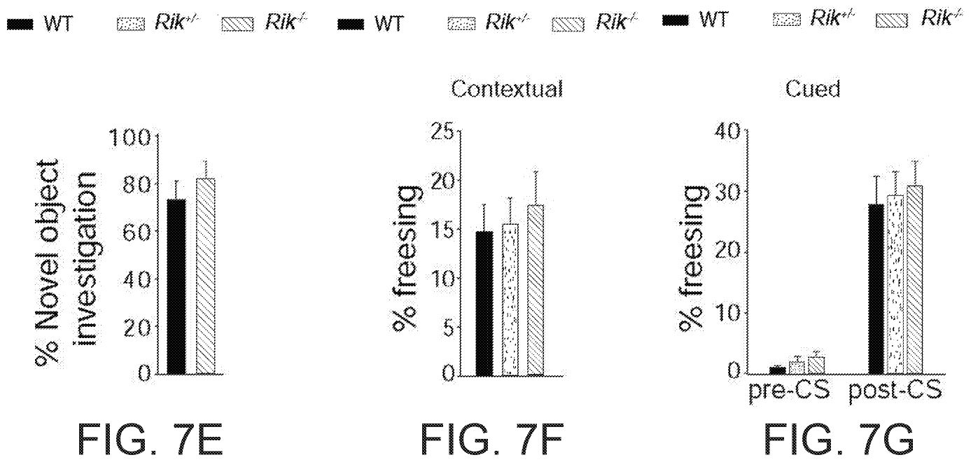

Rik^{+/-} and Rik^{-/-} mice behaved like WT controls in detecting and recognizing nonsocial and social odors (data shown in FIG. 6A), which is crucial for social interaction.¹⁶ Furthermore, both mutants performed at the WT level in the open-field, rotarod, grooming, and elevated plus-maze tests, as shown in FIGS. 6B-6F. Rik^{+/-} and Rik^{-/-} mice also performed comparably to WT mice in hippocampus-dependent memory tasks assayed by the Morris water maze test (data shown in FIGS. 7A-7D), the novel object-recognition test (data shown in FIG. 7E), and fear memory tasks assayed by contextual and cued-fear conditioning tests (data shown in FIGS. 7F and 7G).

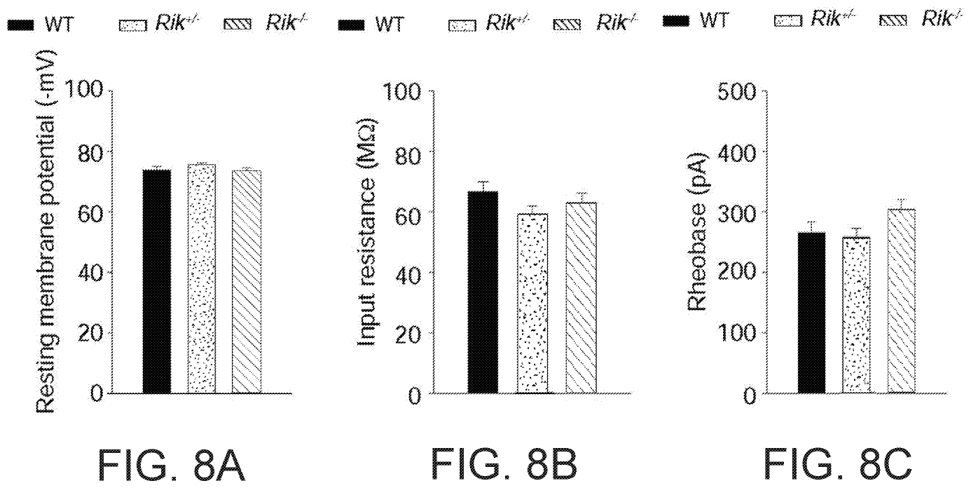

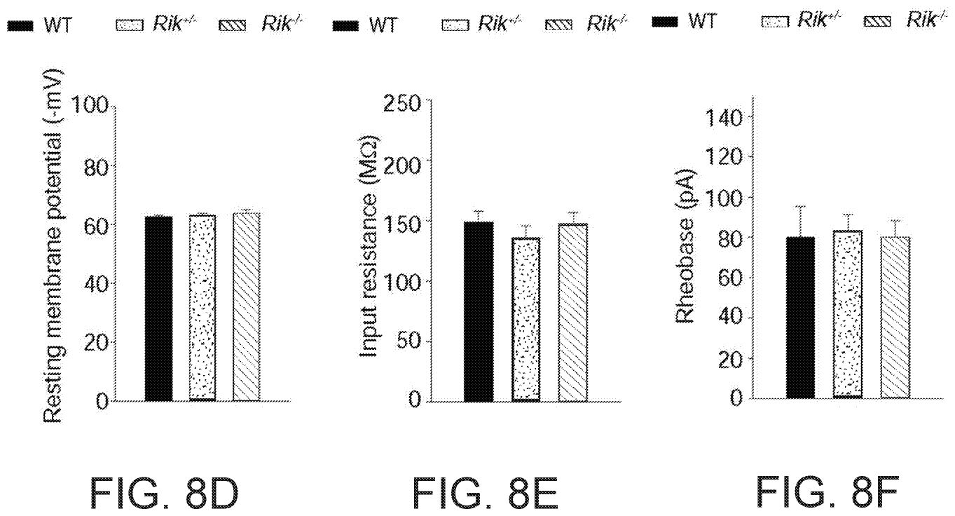

Social memory deficits in a 22q11DS mouse model (Df(16)A^{+/-} mice)¹⁷ were attributed to reduced inhibitory control of the excitatory drive in CA2 pyramidal neurons via reduction of numbers and function of parvalbumin (PV)-positive interneurons in the CA2 area.$ Whole-cell current-clamp experiments in acute hippocampal slices revealed that the intrinsic electrical properties such as resting membrane potential, input resistance, and rheobase of CA2 pyramidal or fast-spiking interneurons did not differ between 2510002D24Rik-deficient mice and WT controls, as shown in FIGS. 8A-8F. However, the inhibitory control at CA3-CA2 excitatory synapses was reduced in 2510002D24Rik-deficient mice. Postsynaptic potentials (PSPs) were recorded in CA2 pyramidal neurons, with or without GABA-receptor antagonists (SR95531 and CGP55845), in response to stimulation of Schaffer collaterals.

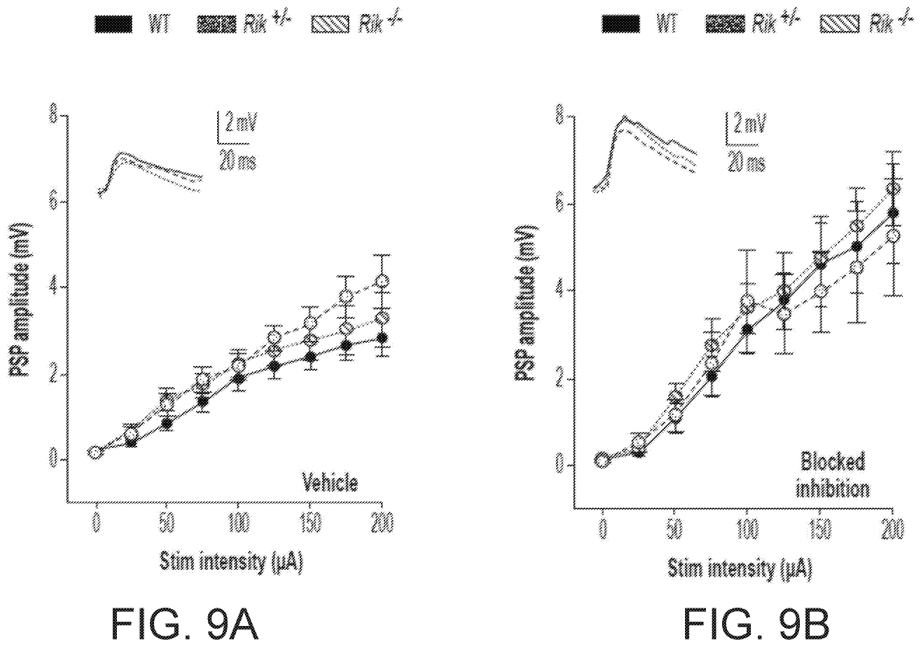

As seen in Df(16)A^{+/-} mice⁸, the PSP peak amplitudes were significantly larger in both mutants than WT controls at basal condition, as shown in FIG. 1C. This difference disappeared in the presence of GABA-receptor antagonists (see FIG. 1D). Similar to what was seen in Df(16)A^{+/-} mice⁸, this increase in PSPs was specific to proximal CA3 inputs and absent in distal inputs from the entorhinal cortex, as shown in FIGS. 9A and 9B. The data of FIG. 1E shows that inhibitory PSPs (IPSPs) were also significantly smaller in 2510002D24Rik-deficient mice than in WT controls. Together, these results suggest that 2510002D24Rik-deficient mice have defective inhibitory control of the excitatory drive in CA2 pyramidal neurons.

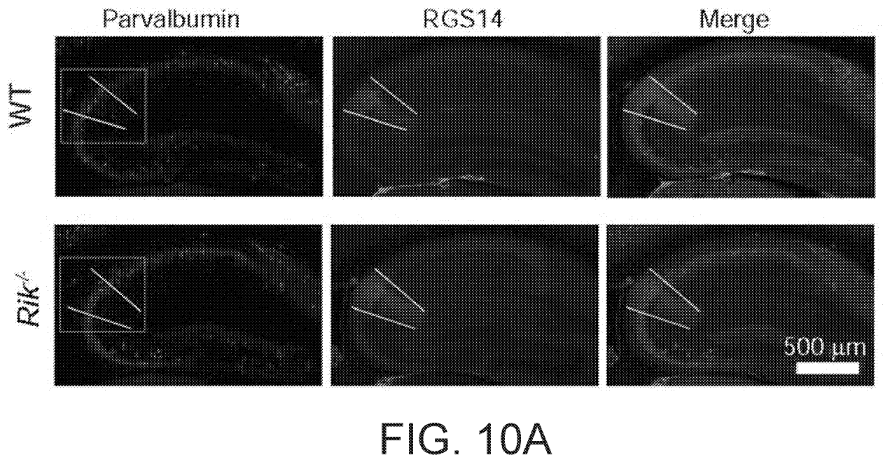

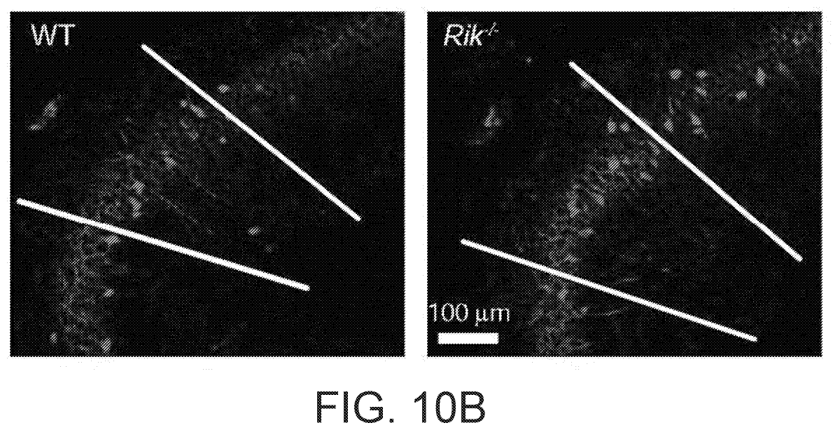

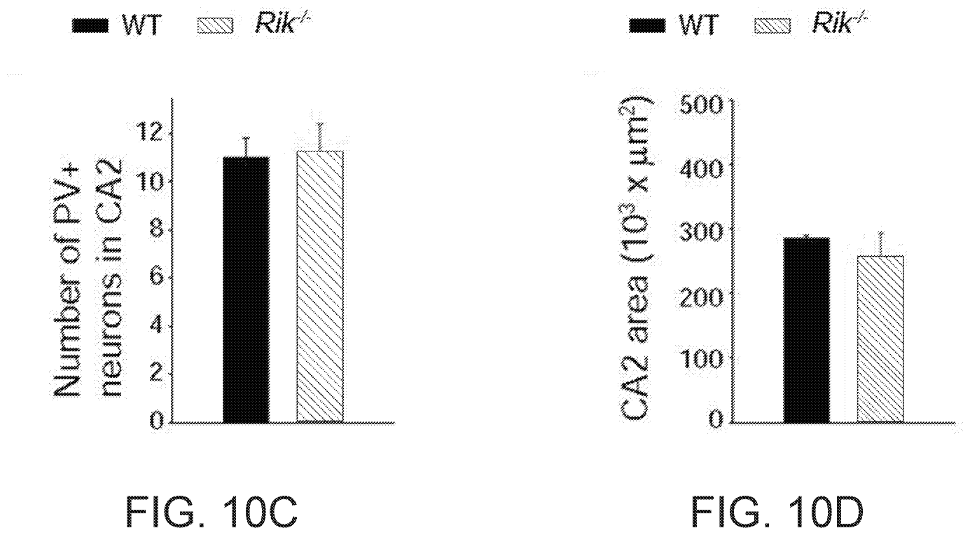

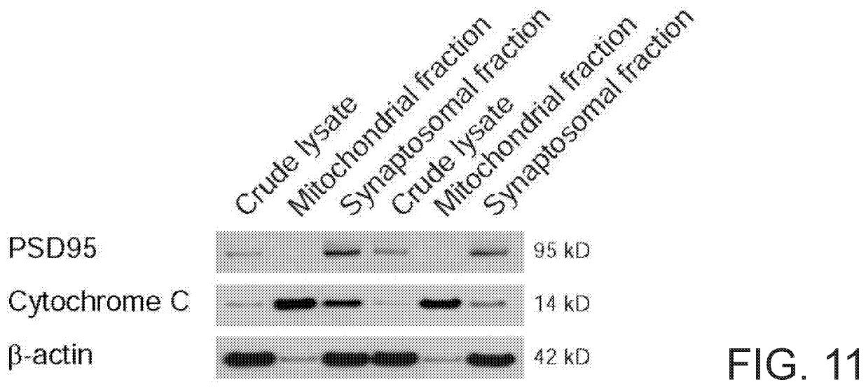

Unlike Df(16)A^{+/-} mice, 2510002D24Rik-deficient mice have a normal number of PV⁺ interneurons in the CA2 area, as shown in FIGS. 10A-10D. However, the CA2 fast-spiking interneurons of 2510002D24Rik-deficient mice fired fewer action potentials (APs) in response to an injection of a depolarizing current step, compared to WT controls. The firing rate of these interneurons was measured by injecting a step current in current-clamp mode. A depolarizing current (150 pA, 1 s) evoked fewer APs in 2510002D24Rik-deficient interneurons than in WT interneurons, as shown in FIG. 1F. This suggests that a decrease in inhibitory control of the excitatory drive in CA2 pyramidal neurons is mediated by the inability of CA2 interneurons to sustain firing of APs.

Inhibitory control is essential for disinhibitory plasticity at CA3-CA2 excitatory synapses. Typically, CA2 pyramidal neurons cannot undergo long-term potentiation¹⁸, but long-term depression at CA3-CA2 inhibitory synapses (CA3-CA2 iLTD) allows for an increase in the net excitatory drive from Schaffer collaterals (CA3 neurons) onto CA2 pyramidal neurons (long-term disinhibitory plasticity).¹⁹ In Df(16)A^{+/-} mice, both CA3-CA2 iLTD and disinhibitory plasticity at CA3-CA2 excitatory synapses were reduced.⁸ Similarly, both CA3-CA2 iLTD and long-term disinhibitory plasticity were impaired in 2510002D24Rik-deficient mice (FIGS. 1C-1H). CA3-CA2 iLTD induced by stimulating the Schaffer collaterals with high-frequency trains (2×100 Hz) was impaired in Rik^{+/-} and Rik^{-/-} mutants compared to WT controls (FIG. 1G). Long-term disinhibitory plasticity in CA2 pyramidal neurons evoked by the same stimulation protocol was also significantly lower in Rik^{+/-} and Rik^{-/-} mice than in WT controls (FIG. 1H).

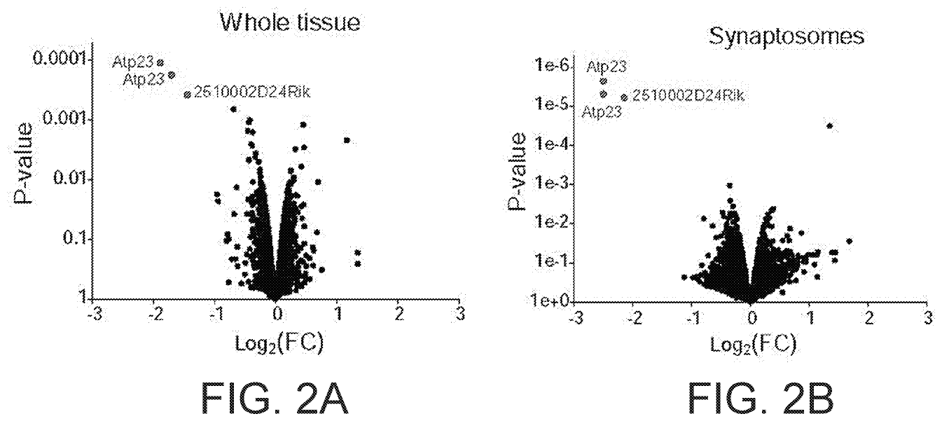

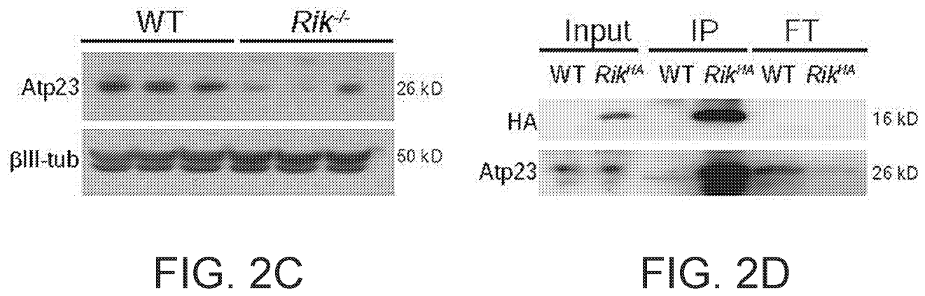

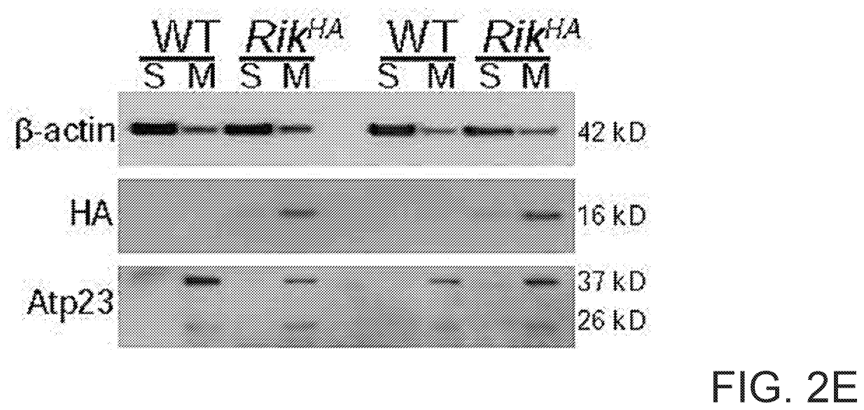

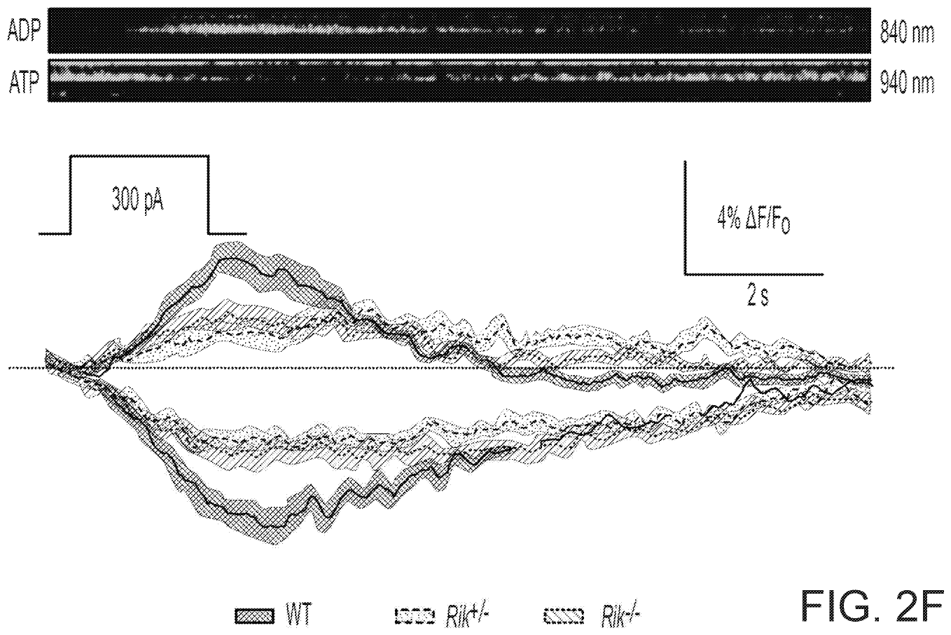

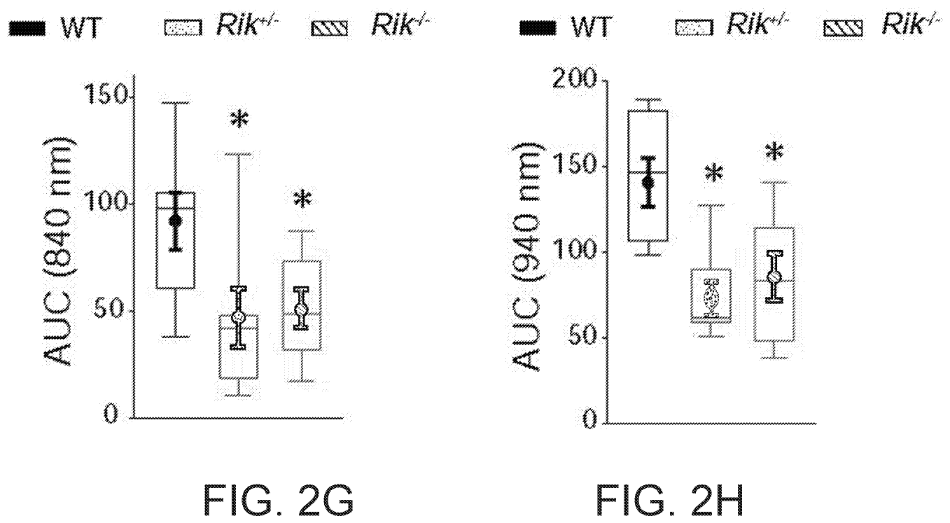

Unbiased proteomics analysis of the whole hippocampus of Rik^{-/-} mice and WT littermates revealed that the 3 most downregulated proteins in the mutants were a polypeptide encoded by 2510002D24Rik and 2 peptides encoded by Atp23 (also known as Xrcc6bp1, Atp23 metallopeptidase, and ATP synthase assembly factor homolog). The data are shown in FIG. 2A and Table 1.

TABLE 1
Proteins in the whole hippocampus of 16-week-old Rik^{-/-}
mice are altered compared to those in WT littermates.

| Gene | Protein Accession # | Log2FC | P-value |
|---|---|---|---|
| Atp23 | sp\|Q9CWQ3\|ATP23_MOUSE | −1.881174 | 0.000111 |
| 2510002D24Rik | tr\|A0A087WSJ3\|A0A087WSJ3_MOUSE | −1.443354 | 0.000374 |
| NA | sp\|P03987\|IGHG3_MOUSE | −0.958906 | 0.017582 |
| Tmem27 | sp\|Q9ESG4\|TMM27_MOUSE | −0.682104 | 0.00066 |
| Sult1c2 | sp\|Q9D939\|ST1C2_MOUSE | −0.631535 | 0.013425 |
| Krt8 | sp\|P11679\|K2C8_MOUSE | −0.454765 | 0.001507 |
| Ide | tr\|F6RPJ9\|F6RPJ9_MOUSE | −0.432014 | 0.004642 |
| Ide | sp\|Q9JHR7\|IDE_MOUSE | −0.430591 | 0.004747 |
| Lgals3 | sp\|P16110\|LEG3_MOUSE | −0.429093 | 0.001087 |
| Kcnj13 | sp\|P86046\|KCJ13_MOUSE | −0.422139 | 0.000994 |
| Agt | sp\|P11859\|ANGT_MOUSE | −0.393425 | 0.002566 |
| Grk5 | sp\|Q8VEB1\|GRK5_MOUSE | −0.379441 | 0.019111 |
| Adh1 | sp\|P00329\|ADH1_MOUSE | −0.378093 | 0.002681 |
| Tmem72 | sp\|Q8C3K5\|TMM72_MOUSE | −0.37074 | 0.001613 |
| Itgb4 | sp\|A2A863\|ITB4_MOUSE | −0.367451 | 0.010811 |
| Gch1 | sp\|Q05915\|GCH1_MOUSE | −0.331395 | 0.004176 |
| Junb | sp\|P09450\|JUNB_MOUSE | −0.321878 | 0.003634 |
| Arhgdib | sp\|Q61599\|GDIR2_MOUSE | −0.309459 | 0.018167 |
| Myo7a | sp\|P97479\|MYO7A_MOUSE | −0.30626 | 0.016237 |
| Nudt3 | sp\|Q9JI46\|NUDT3_MOUSE | −0.271961 | 0.005056 |
| Cgn | sp\|P59242\|CING_MOUSE | −0.255429 | 0.007692 |
| Galm | sp\|Q8K157\|GALM_MOUSE | −0.251655 | 0.006792 |
| Vat1l | sp\|Q80TB8\|VAT1L_MOUSE | −0.247896 | 0.011458 |
| Spata18 | sp\|Q0P557\|MIEAP_MOUSE | −0.246716 | 0.018788 |
| Slc25a1 | sp\|Q8JZU2\|TXTP_MOUSE | −0.246431 | 0.009027 |
| Gm11992 | sp\|Q5SS90\|CG057_MOUSE | −0.243045 | 0.010784 |
| Ptpn14 | sp\|Q62130\|PTN14_MOUSE | −0.235317 | 0.014325 |
| Pcdhb16 | tr\|Q91Y03\|Q91Y03_MOUSE | −0.231047 | 0.016708 |
| Atg5 | sp\|Q99J83\|ATG5_MOUSE | −0.229725 | 0.012045 |
| Sema3b | sp\|Q62177\|SEM3B_MOUSE | −0.224553 | 0.012053 |
| Pus7 | tr\|Q91VU7\|Q91VU7_MOUSE | −0.221852 | 0.01531 |
| Hfe | sp\|P70387\|HFE_MOUSE | −0.216421 | 0.019498 |
| Shq1 | sp\|Q7TMX5\|SHQ1_MOUSE | −0.214661 | 0.012722 |
| Klhdc4 | tr\|G3X961\|G3X961_MOUSE | −0.208328 | 0.014303 |
| Crb2 | sp\|Q80YA8\|CRUM2_MOUSE | −0.2068 | 0.019946 |
| Wscd1 | sp\|Q80XH4\|WSCD1_MOUSE | −0.206734 | 0.01736 |
| Chuk | sp\|Q60680\|IKKA_MOUSE | −0.205388 | 0.01878 |
| Thrsp | sp\|Q62264\|THRSP_MOUSE | −0.203686 | 0.015395 |
| Oprm1 | sp\|P42866\|OPRM_MOUSE | −0.203028 | 0.014209 |
| Ttc25 | sp\|Q9D4B2\|TTC25_MOUSE | −0.202911 | 0.013687 |
| Pcdhb11 | tr\|Q91UZ8\|Q91UZ8_MOUSE | −0.202469 | 0.013802 |
| Sars2 | sp\|Q9JJL8\|SYSM_MOUSE | −0.190878 | 0.018022 |
| Tspyl4 | sp\|Q8VD63\|TSYL4_MOUSE | −0.182806 | 0.018933 |
| Kpna2 | sp\|P52293\|IMA1_MOUSE | 0.19299 | 0.016252 |
| Pak6 | sp\|Q3ULB5\|PAK6_MOUSE | 0.19591 | 0.019609 |
| P4htm | sp\|Q8BG58\|P4HTM_MOUSE | 0.19597 | 0.015938 |
| Itm2a | sp\|Q61500\|ITM2A_MOUSE | 0.20647 | 0.014504 |
| Gm16494 | tr\|E9Q2Z4\|E9Q2Z4_MOUSE | 0.21461 | 0.012472 |
| Sts | sp\|P50427\|STS_MOUSE | 0.21593 | 0.014673 |
| Plekhd1 | sp\|B2RPU2\|PLHD1_MOUSE | 0.21844 | 0.017192 |
| Med24 | sp\|Q99K74\|MED24_MOUSE | 0.22488 | 0.016817 |
| Grpel2 | sp\|O88396\|GRPE2_MOUSE | 0.24109 | 0.01104 |
| Rnf146 | sp\|Q9CZW6\|RN146_MOUSE | 0.24268 | 0.011273 |
| Atxn2 | tr\|F6V8M6\|F6V8M6_MOUSE | 0.24786 | 0.007166 |
| Yjefn3 | sp\|F6W8I0\|YJEN3_MOUSE | 0.25565 | 0.00703 |
| H1f0 | sp\|P10922\|H10_MOUSE | 0.25935 | 0.011419 |
| Zfp37 | sp\|P17141\|ZFP37_MOUSE | 0.25956 | 0.018573 |
| Zscan21 | sp\|Q07231\|ZSC21_MOUSE | 0.26003 | 0.011638 |
| Itga1 | sp\|Q3V3R4\|ITA1_MOUSE | 0.29782 | 0.01023 |
| Itga3 | sp\|Q62470\|ITA3_MOUSE | 0.2981 | 0.00918 |
| Fam219b | sp\|Q14DQ1\|F219B_MOUSE | 0.32615 | 0.003074 |
| Sypl2 | sp\|O89104\|SYPL2_MOUSE | 0.32904 | 0.018114 |
| Chic1 | sp\|Q8CBW7\|CHIC1_MOUSE | 0.35784 | 0.018756 |
| Zkscan16 | tr\|A2ALW2\|A2ALW2_MOUSE | 0.42337 | 0.005996 |
| Cox17 | sp\|P56394\|COX17_MOUSE | 0.45677 | 0.001197 |
| Neu3 | sp\|Q9JMH7\|NEUR3_MOUSE | 0.46695 | 0.002894 |
| Gm4975 | tr\|J3QMK1\|J3QMK1_MOUSE | 0.6986 | 0.01093 |
| Efcab7 | sp\|Q8VDY4\|EFCB7_MOUSE | 1.16573 | 0.002172 |
| Tmem126b | sp\|Q9D1R1\|T126B_MOUSE | 1.19056 | 3.93E−05 |
| Nnt | sp\|Q61941\|NNTM_MOUSE | 2.35469 | 9.43E−06 |

The same three proteins were also the most downregulated in validated synaptosomal fractions (see FIG. 11) isolated from the hippocampi of Rik^{-/-} and WT mice, as shown in FIG. 2B and Table 2. Western-blot analysis confirmed that Atp23 is substantially reduced in Rik^{-/-} mice, as shown in FIG. 2C.

TABLE 2
Altered proteins in synaptosomes extracted from the whole hippocampus
of 16-week old Rik^{-/-} mice compared to WT littermates.

| Gene | Protein Accession # | Log₂FC | p-value |
|---|---|---|---|
| Atp23 | sp\|Q9CWQ3\|ATP23_MOUSE | −2.49129 | 4.93697E−06 |
| 2510002D24Rik | tr\|A0A087WSJ3\|A0A087WSJ3_MOUSE | −2.135615 | 6.13385E−06 |
| Klhdc10 | sp\|Q6PAR0\|KLD10_MOUSE | −0.786107 | 0.007443791 |
| Myo15a | sp\|Q9QZZ4\|MYO15_MOUSE | −0.637775 | 0.011584436 |
| Ces1 | sp\|Q8VCC2\|EST1_MOUSE | −0.464792 | 0.005176465 |
| Psrc1 | sp\|Q9D0P7\|PSRC1_MOUSE | −0.452784 | 0.006437234 |
| Caln1 | sp\|Q9JJG7\|CABP8_MOUSE | −0.429955 | 0.017719806 |
| Mxra7 | sp\|Q9CZH7\|MXRA7_MOUSE | −0.402925 | 0.018451731 |
| Dsp | sp\|E9Q557\|DESP_MOUSE | −0.402163 | 0.013154723 |
| Nod2 | sp\|Q8K3Z0\|NOD2_MOUSE | −0.401952 | 0.012247843 |
| Cfap58 | tr\|B2RW38\|B2RW38_MOUSE | −0.390568 | 0.010277119 |
| Pigu | sp\|Q8K358\|PIGU_MOUSE | −0.378273 | 0.019258143 |
| Cdc42ep2 | sp\|Q8JZX9\|BORG1_MOUSE | −0.377048 | 0.007201631 |
| Senp5 | sp\|Q6NXL6\|SENP5_MOUSE | −0.351623 | 0.013507216 |
| Cd44 | sp\|P15379-10\|CD44_MOUSE | −0.346483 | 0.016043442 |
| Gnptab | sp\|Q69ZN6\|GNPTA_MOUSE | −0.343222 | 0.001065776 |
| Klhl2 | sp\|Q8JZP3\|KLHL2_MOUSE | −0.333578 | 0.002589927 |
| Cystm1 | sp\|Q8K353\|CYTM1_MOUSE | −0.33277 | 0.005635468 |
| Lman2l | sp\|P59481\|LMA2L_MOUSE | −0.330438 | 0.00653487 |
| Casd1 | sp\|Q7TN73\|CASD1_MOUSE | −0.307547 | 0.013714455 |
| Rhou | sp\|Q9EQT3\|RHOU_MOUSE | −0.300097 | 0.016850365 |
| Ubxn4 | tr\|A0A087WSK5\|A0A087WSK5_MOUSE | −0.29182 | 0.003650985 |
| Btbd3 | sp\|P58545\|BTBD3_MOUSE | −0.289489 | 0.003578013 |
| Ypel3 | sp\|P61237\|YPEL3_MOUSE | −0.286072 | 0.009207956 |
| Gc | sp\|P21614\|VTDB_MOUSE | −0.275213 | 0.019945805 |
| Hspa13 | sp\|Q8BM72\|HSP13_MOUSE | −0.274189 | 0.009343557 |
| Arhgap42 | sp\|B2RQE8\|RHG42_MOUSE | −0.262866 | 0.00548731 |
| Samd8 | sp\|Q9DA37\|SAMD8_MOUSE | −0.256451 | 0.01427959 |
| Slco3a1 | sp\|Q8R3L5\|SO3A1_MOUSE | −0.256172 | 0.014865082 |
| Kcnk4 | sp\|O88454\|KCNK4_MOUSE | −0.24736 | 0.015664998 |
| Tor2a | sp\|Q8R1J9\|TOR2A_MOUSE | −0.244306 | 0.018760002 |
| Sumf2 | sp\|Q8BPG6\|SUMF2_MOUSE | −0.242812 | 0.016631124 |
| D17h6s53e | sp\|Q9Z1R4\|CF047_MOUSE | −0.240027 | 0.00661586 |
| Gigyf2 | sp\|Q6Y7W8\|PERQ2_MOUSE | −0.239663 | 0.012603122 |
| Cerk | sp\|Q8K4Q7\|CERK1_MOUSE | −0.231491 | 0.012358048 |
| Ephx4 | sp\|Q6IE26\|EPHX4_MOUSE | −0.226445 | 0.012088793 |
| Tyk2 | sp\|Q9R117\|TYK2_MOUSE | −0.225768 | 0.007738916 |
| Dhrs13 | sp\|Q5SS80\|DHR13_MOUSE | −0.22231 | 0.013625179 |
| Nfx1 | sp\|B1AY10\|NFX1_MOUSE | −0.204414 | 0.018345683 |
| Dennd2a | sp\|Q8C4S8\|DEN2A_MOUSE | −0.201213 | 0.018636601 |
| Qsox2 | sp\|Q3TMX7\|QSOX2_MOUSE | −0.200757 | 0.018927503 |
| Rnf181 | sp\|Q9CY62\|RN181_MOUSE | −0.196052 | 0.018210542 |
| Tifa | sp\|Q793I8\|TIFA_MOUSE | 0.20417 | 0.017490009 |
| Cldn12 | sp\|Q9ET43\|CLD12_MOUSE | 0.21609 | 0.013606468 |
| Mansc1 | sp\|Q9CR33\|MANS1_MOUSE | 0.2331 | 0.012257618 |
| Ace | sp\|P09470\|ACE_MOUSE | 0.23549 | 0.012272275 |
| Slc32a1 | sp\|O35633\|VIAAT_MOUSE | 0.23608 | 0.018205318 |
| Trim26 | sp\|Q99PN3\|TRI26_MOUSE | 0.24838 | 0.010402106 |
| Ppp1r2 | sp\|Q9DCL8\|IPP2_MOUSE | 0.27392 | 0.007511401 |
| Plg | sp\|P20918\|PLMN_MOUSE | 0.29352 | 0.008522427 |
| Cryba4 | sp\|Q9JJV0\|CRBA4_MOUSE | 0.30859 | 0.005796556 |
| Lgals1 | sp\|P16045\|LEG1_MOUSE | 0.32733 | 0.007072827 |
| Enpp3 | sp\|Q6DYE8\|ENPP3_MOUSE | 0.33051 | 0.011514242 |
| Pmch | sp\|P56942\|MCH_MOUSE | 0.36459 | 0.00464163 |
| Hmgcs2 | sp\|P54869\|HMCS2_MOUSE | 0.37081 | 0.01189837 |
| Ppp1r14b | sp\|Q62084\|PP14B_MOUSE | 0.37357 | 0.004635838 |
| Mthfd2 | sp\|P18155\|MTDC_MOUSE | 0.39935 | 0.0041522 |
| Hint1 | sp\|P70349\|HINT1_MOUSE | 0.67922 | 0.013271695 |
| Hebp1 | sp\|Q9R257\|HEBP1_MOUSE | 0.87191 | 0.017214249 |
| Tmem126b | sp\|Q9D1R1\|T126B_MOUSE | 1.35616 | 3.20321E−05 |

The proteomics data are deposited in the NCBI GEO database under accession number PXD013989, and is incorporated by reference herein in its entirety.

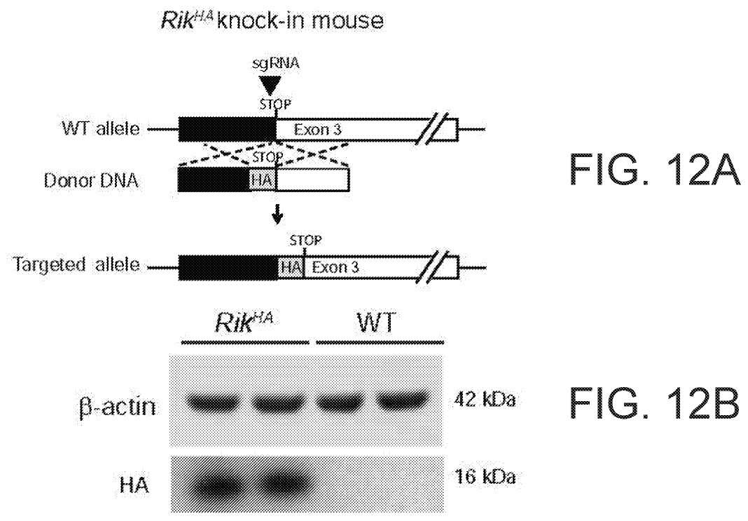

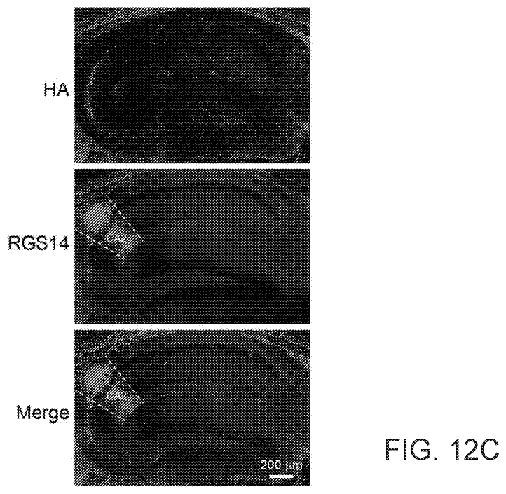

An assay was undertaken to determine if 2510002D24Rik and Atp23 interact. Small proteins such as 2510002D24Rik (105-amino acids) generally show poor immunoreactivity, and antibodies against them are not effective in immunoprecipitation experiments. Accordingly, the CRISPR/Cas9 approach was used to produce a mutant mouse with 2510002D24Rik knocked in and fused with a human influenza hemagglutinin (HA) tag. HA was reliably detected in homozygous knock-in mice (Rik^{HA} mice) but was absent in WT mice (see FIG. 12B). The data of FIG. 2D shows that Atp23 co-precipitated with HA in Rik^{HA} mice, suggesting that the 2 proteins are in a complex. Because Atp23 is a mitochondrial intermembrane space protein and is required for the maturation of the mitochondrially encoded F₀-subunit Atp6 and its assembly into the F₁F₀-ATP synthase complex²⁰⁻²², an assay was performed to determine if 2510002D24Rik is also localized to the mitochondria. HA was substantially enriched in the mitochondrial fraction, compared to synaptosomal fraction from Rik^{HA} hippocampal homogenates, and was absent in both WT fractions (as shown in FIG. 2E). These results suggest that 2510002D24Rik is a mitochondrial protein, consistent with that 2510002D24Rik amino acid sequence contains 2 CX₉C domains characteristic of mitochondrial intermembrane space proteins.²³

The interaction between 2510002D24Rik and Atp23 suggested that these proteins affect ATP equilibrium, which is essential for proper neural function²⁴ and especially important for fast-spiking interneurons that use excessive energy.¹³ A recombinant adeno-associated virus (AAV) encoding the fluorescent ATP/ADP sensor PercevalHR²⁵, under control of the neuron-specific promoter hSynapsin (AAV-hSyn-PercevalHR), was used to measure ATP and ADP in individual neurons in hippocampal slices from adult mice. Several weeks after the injections of this virus into the CA2 area, recordings of fluorescently labelled interneurons or pyramidal neurons were made by using 2-photon imaging and whole-cell current-clamp recordings. A depolarizing current (300 pA, 2 s) injected into a cell body of labelled fast-spiking interneurons produced an increase in ADP (measured by fluorescence excited at 840 nm) and a decrease in ATP (measured by fluorescence excited at 940 nm). The data is shown in FIG. 2F.

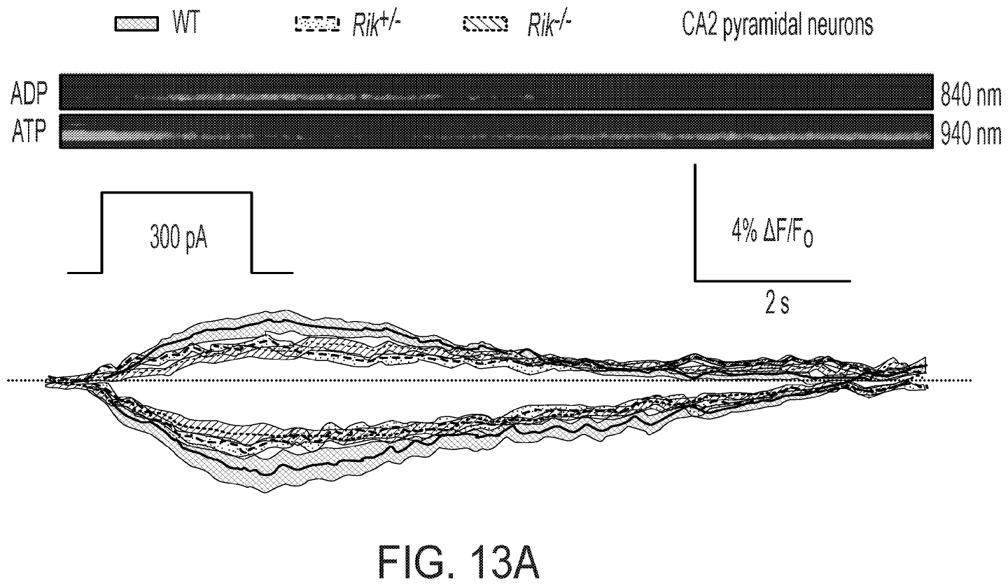

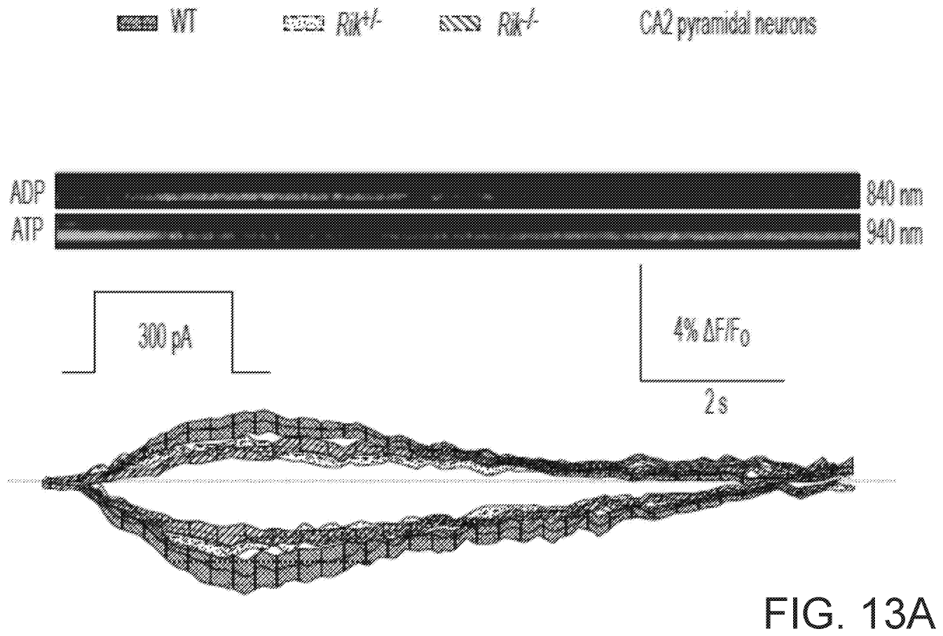

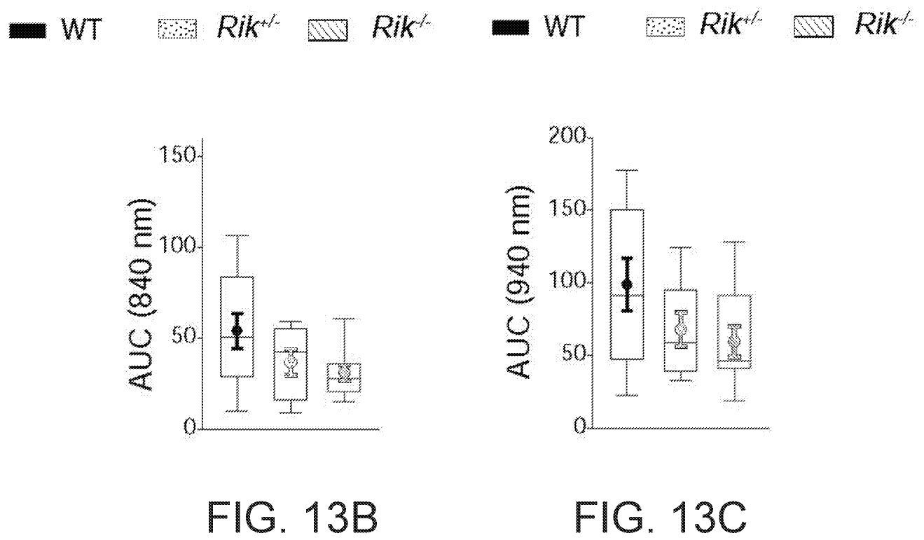

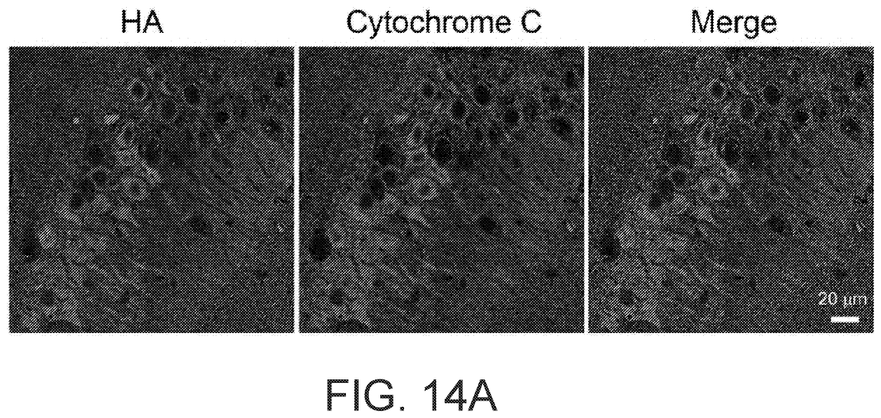

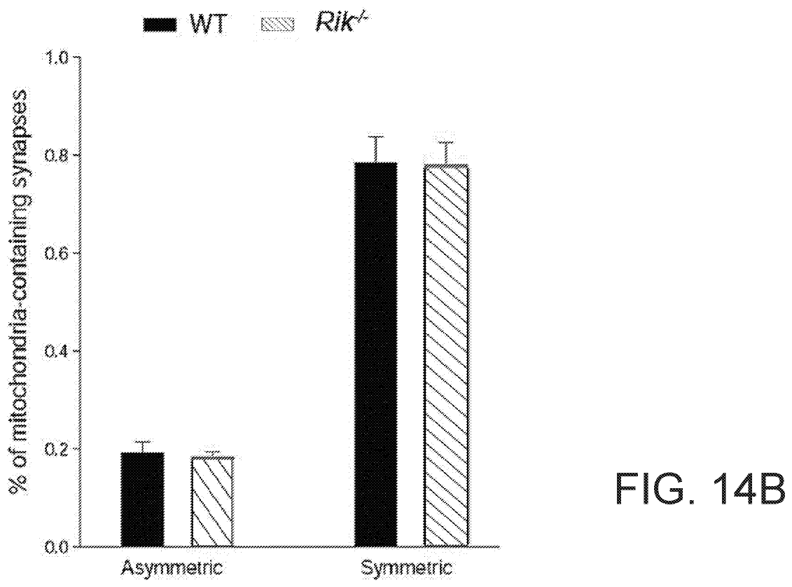

Compared to WT CA2 interneurons, Rik^{+/-} and Rik^{-/-} CA2 interneurons showed smaller changes in ATP and ADP levels during the evoked train of APs (see FIG. 2H), indicating reduced interconversion of ATP and ADP during the rapid firing in mutant CA2 interneurons. In comparison, activity-dependent increase in ATP and ADP was not significantly different between genotypes in CA2 pyramidal neurons, as shown in FIGS. 13A-13C. Three-dimensional (3D) electron microscopy of the CA2 area revealed that approximately 80% of inhibitory (symmetric) synapses contained mitochondria, but only about 20% of excitatory (asymmetric) synapses did. See FIG. 14B. These ratios were not different between the genotypes, suggesting that functional (rather than structural) mitochondrial deficits underlie deficient ATP availability in CA2 interneurons of 2510002D24Rik-deficient mice.

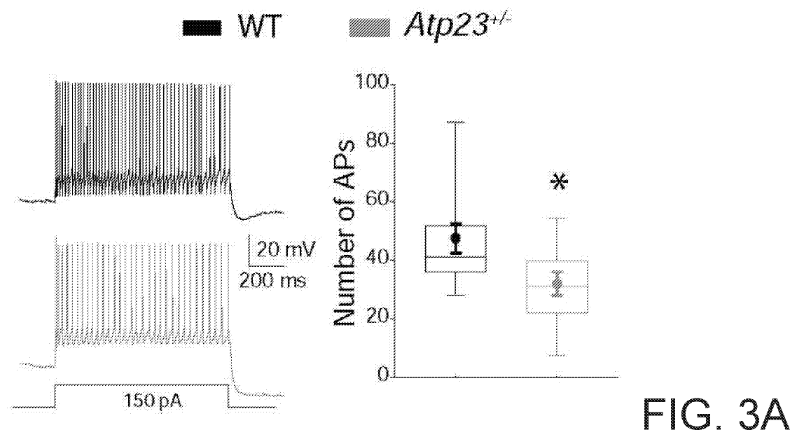

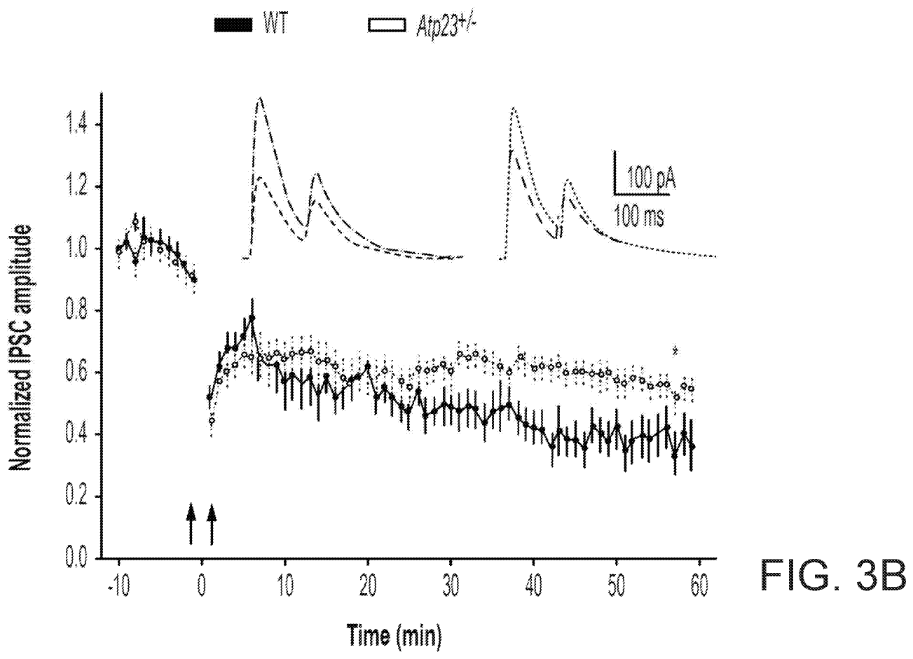

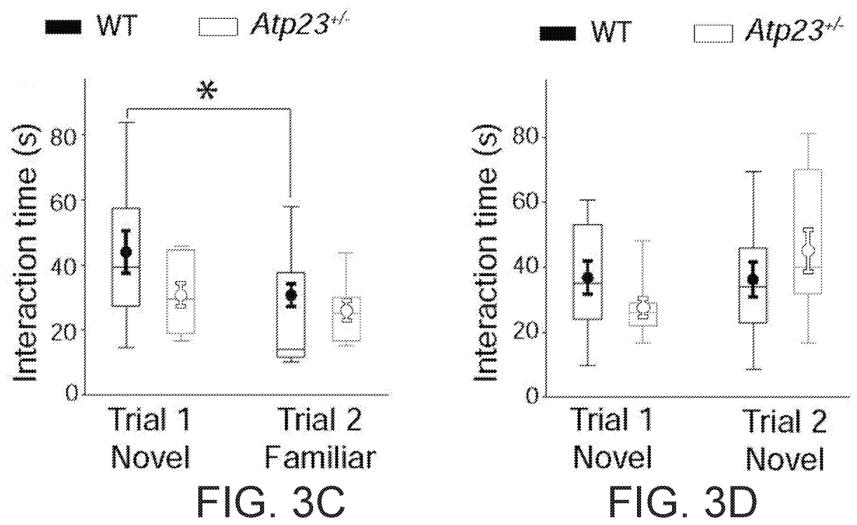

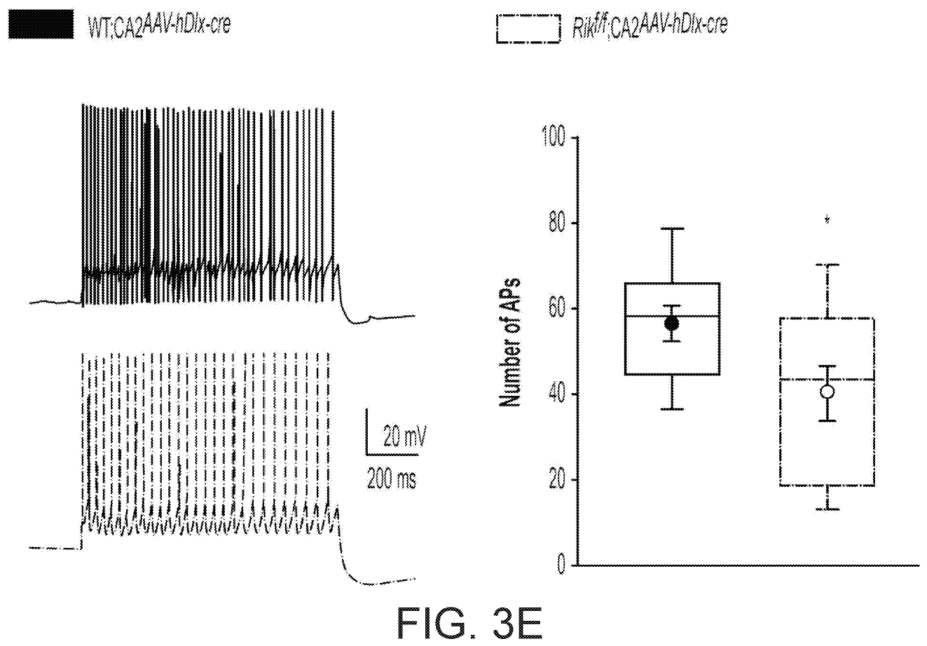

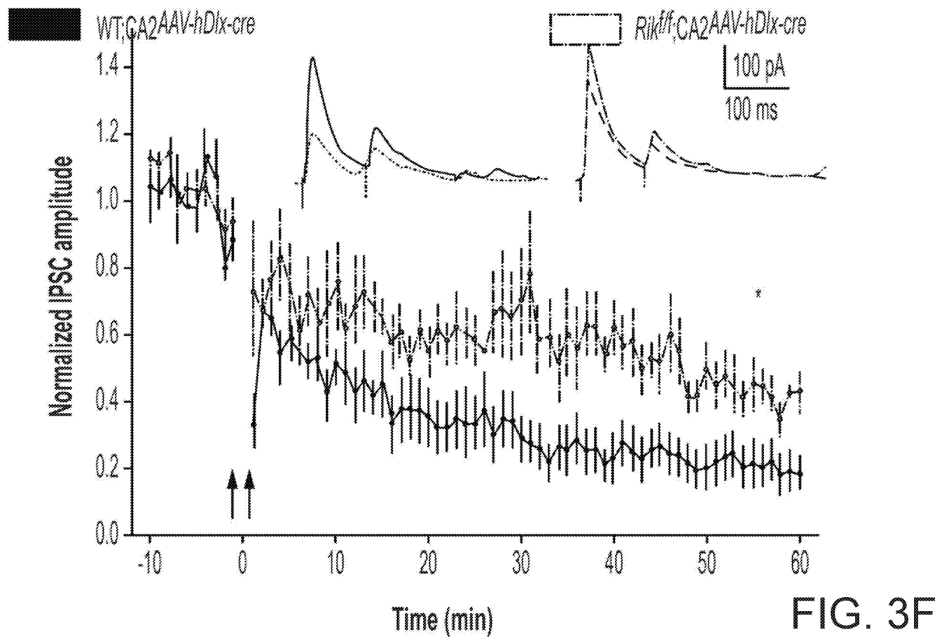

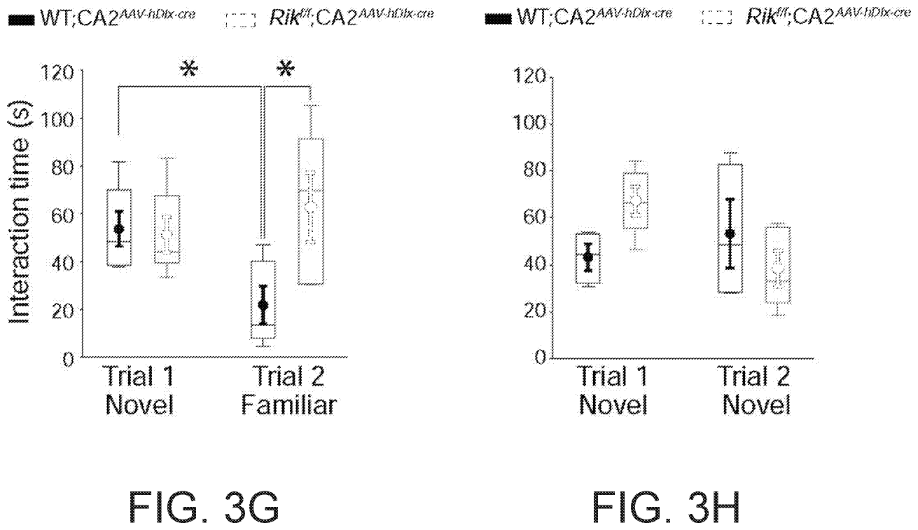

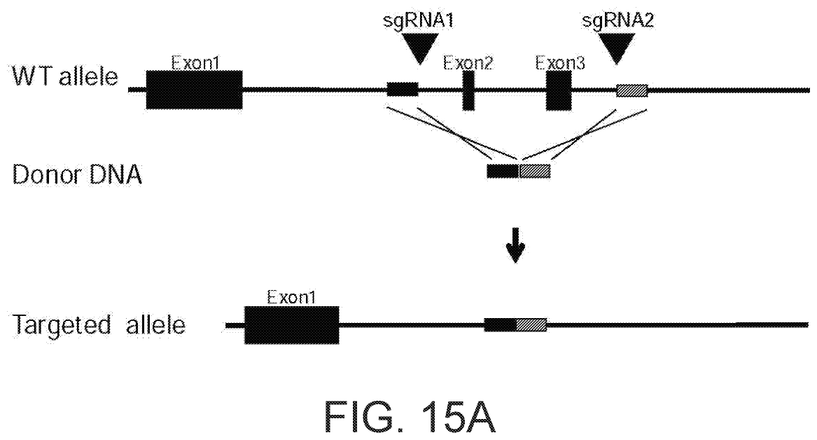

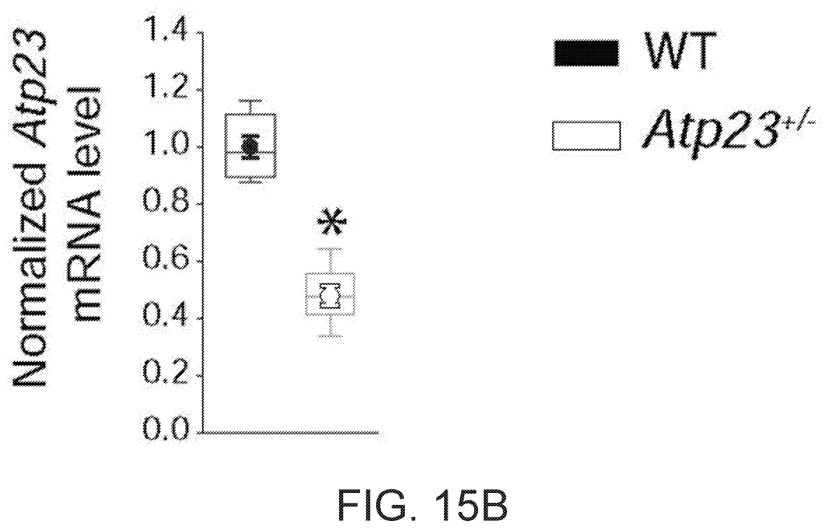

CRISPR/Cas9-mediated depletion of Atp23 resulted in phenocopying of CA2 neuronal, synaptic, and behavioral abnormalities of 2510002D24Rik-deficient mice. Atp23^{-/-} mice were embryonically lethal, and Atp23+/− mice developed normally but showed a reduction of the Atp23 transcript by about 50% (see FIG. 15B). Atp23^{+/-} mice had reduced firing of APs in CA2 interneurons and reduced CA3-CA2 iLTD, as shown in FIGS. 3A and 38. These mutants showed no reduction in interaction time from a novel to a familiar mouse in the one-chamber direct-interaction (social recognition) test, whereas their WT littermates did (as shown in FIG. 3C). Atp23^{+/-} mice performed similarly to WT mice during trials 1 and 2, when 2 different novel mice were encountered (as shown in FIG. 3D).

Social memory deficit was also caused by knocking down 2510002D24Rik only in CA2 interneurons. AAVs expressing cre recombinase (cre) and GFP, under control of the human form of the Dlx5/6 enhancer (hDlx)², which restricts expression to GABAergic interneurons, were injected into the CA2 area (CA2^{AVV-hDlx-cre}) of newly developed mutant mice carrying the floxed 2510002D24Rik alleles (Rik^{f/f} mice). GFP⁺ fast-spiking interneurons fired fewer APs after depolarization, and iLTD recorded from CA2 pyramidal neurons was reduced in Rik^{f/f};CA2^{AVV-hDlx-cre} mice compared to WT;CA2^{AVV-hDlx-cre} mice, as shown in FIGS. 3E and 3F. Furthermore, Rik^{f/f};CA2^{AVV-hDlx-cre} mice performed worse than WT;CA2^{AVV-hDlx-cre} mice in the one-chamber social-recognition test but not the sociability test. See FIGS. 3G and 3H.

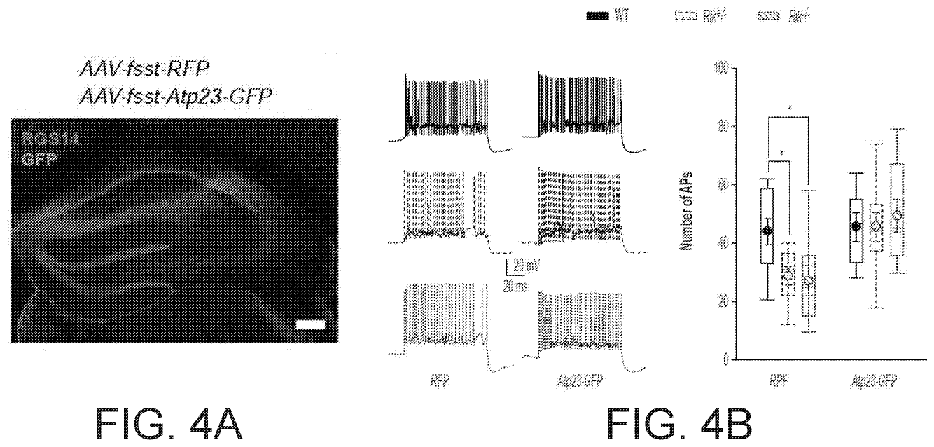

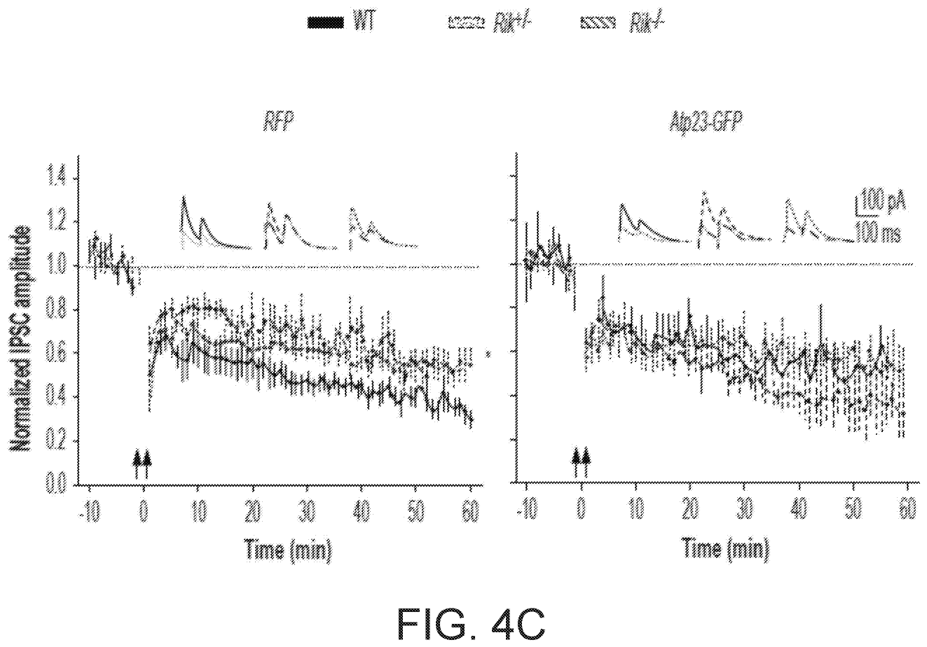

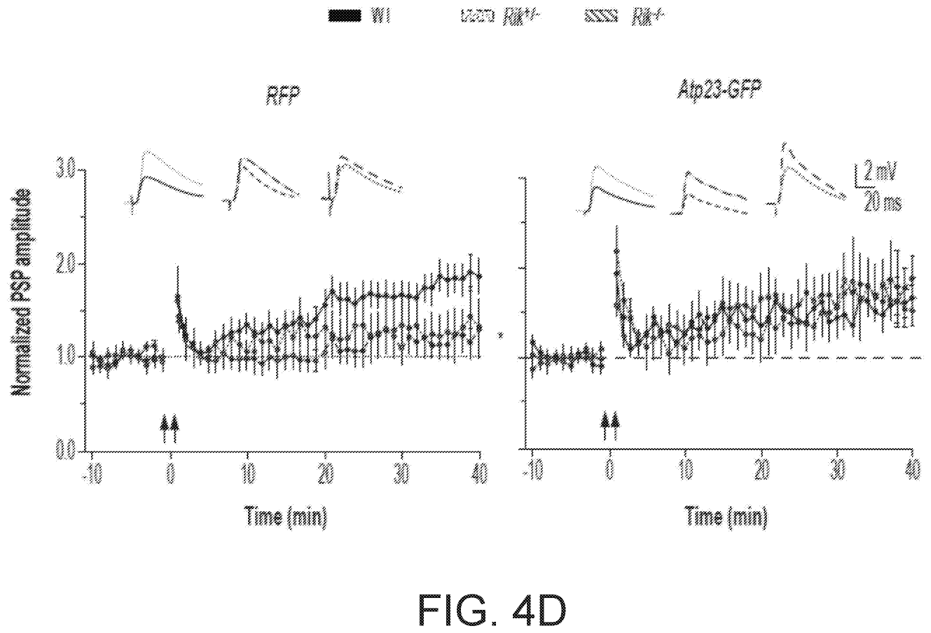

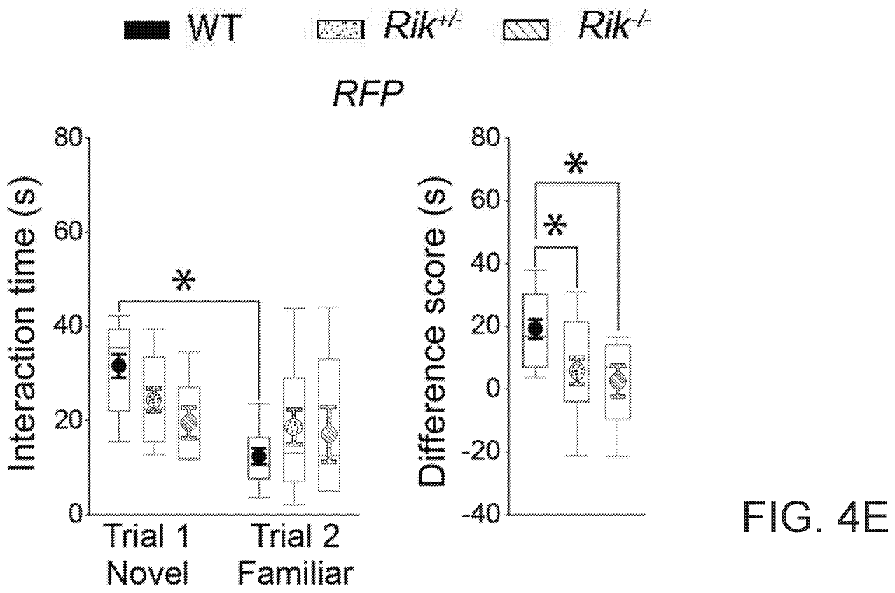

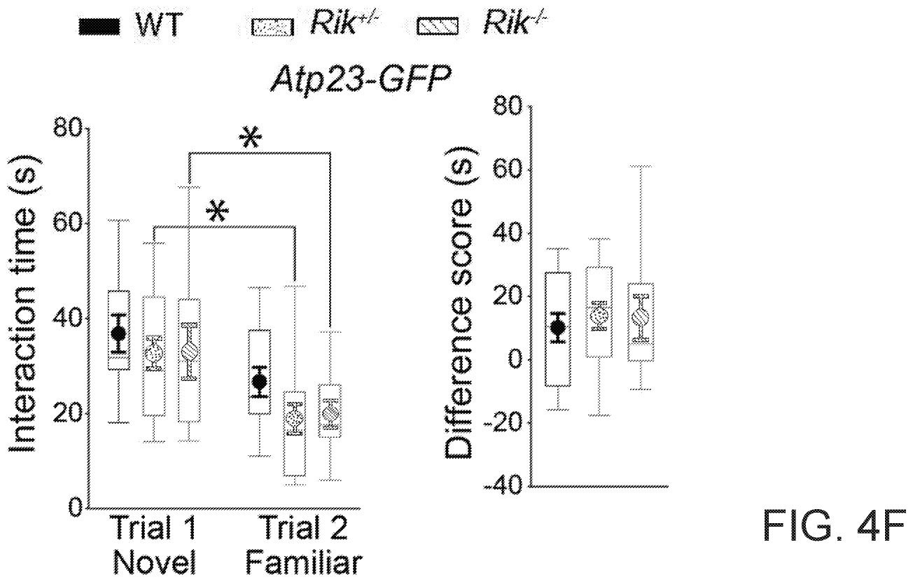

An attempt to rescue neural, synaptic, and social memory abnormalities was made by replenishing Atp23 in the CA2 interneurons of 2510002D24Rik-deficient mice. AAVs expressing Atp23 fused with GFP or red fluorescent protein (RFP) alone, under control of fsst, a pan-GABAergic interneuron promoter²⁷, were injected into the CA2 area (CA2^{AVV-fsst-Atp23} or CA2^{AVV-fsst-RFP}) of WT, Rik^{+/-}, and Rik^{-/-} mice (as shown in FIG. 4A). Recording from fluorescently labelled fast-spiking cells revealed that Atp23 overexpression but not RFP increased firing of APs in CA2 2510002D24Rik-deficient CA2 interneurons to the WT levels without affecting firing in WT interneurons (as shown in FIG. 4B). FIG. 4C shows that CA3-CA2 iLTD was also rescued and expressed at WT levels when both mutants were injected with CA2^{AVV-fsst-Atp23} but not CA2^{AVV-fsst-RFP}. Long-term disinhibition of CA2 pyramidal neurons was also restored to the WT level in mutants injected with CA2^{AVV-fsst-Atp23} but not CA2^{AVV-fsst-RFP} (as shown in FIG. 4D). These results suggest that deficient ADP/ATP interconversion caused by Atp23 depletion underlies neural and synaptic abnormalities in 2510002D24Rik-deficient mice. AAV-mediated overexpression of ATP23 also rescued the social-recognition deficit (see FIG. 4E), but a control AAV did not.

2510002D24Rik was identified herein as a major contributor to hippocampal CA2-dependent social memory deficiency associated with 22q11DS. Interaction between 2 mitochondrial proteins encoded by nuclear genes 2510002D24Rik and Atp23 underlies reduced firing of APs in CA2 fast-spiking interneurons and subsequent reduction in CA3-CA2 iLTD and disinhibitory drive at CA3-CA2 synapses. This causes deficient social memory, presumably due to a reduced output from CA2 to the hypothalamus, ventral CA1 area, and other regions involved in social memory.^{2,28-30} 2510002D24Rik deficiency-mediated energy imbalance in fast-spiking interneurons, which require high energy expenditure¹³ and are particularly numerous in the CA2 area¹⁴, makes CA2 interneurons especially vulnerable. At least 7 other genes that encode mitochondrial proteins within the 22q11.2 region³¹ could exacerbate this metabolic vulnerability and cause interneuron-specific cell death³², as seen in mouse models of 27-gene 22q11DS microdeletion.⁸ Social dysfunction is a major symptom of many neuropsychiatric and neurologic diseases^{33,34}, and the CA2 region plays a crucial role in sociocognitive processing.^{35,33}

Sequences
UPF0545 protein isoform 1 encoded by human
2510002D24Rik gene (also known as Human gene
C22orf39):
SEQ ID NO: 1
MCRCSLVLLSVDHEVPFSSFFIGWRTEGRAWRAGRPDMADGSGWQPPRPC
EAYRAEWKLCRSARHFLHHYYVHGERPACEQWQRDLASCRDWEERRNAEA
QQSLCESERARVRAARKHILVWAPRQSPPPDWHLPLPQEKDE
protein encoded by human Atp23 gene
SEQ ID NO: 2
MAGAPDERRRGPAAGEQLQQQHVSCQVFPERLAQGNPQQGFFSSFFTSNQ
KCQLRLLKTLETNPYVKLLLDAMKHSGCAVNKDRHFSCEDCNGNVSGGFD
ASTSQIVLCQNNIHNQAHMNRVVTHELIHAFDHCRAHVDWFTNIRHLACS
EVRAANLSGDCSLVNEIFRLHFGLKQHHQTCVRDRATLSILAVRNISKEV
AKKAVDEVFESCFNDHEPFGRIPHNKTYARYAHRDFENRDRYYSNI
protein encoded by mouse 2510002D24Rik gene (also
known as UPF0545 protein C22orf39 homolog)
SEQ ID NO: 3
MAVAGSWQPPRPCEVYRAEWELCRSVGHVLHHYYVHGKRPDCRQWLRDLT
NCREWEESRSAEAQRSLCESEQVRVQAAQKHTLVWALRQRPPTDWNLPLP
QEKDK
protein encoded by mouse Atp23 gene (Uniprot:
G3UW46)
SEQ ID NO: 4
MAGAPGGGELGPAAGEPLLQRPDSGQGSPEPPAHGKPQQGFLSSLFTRDQ
SCPLMLQKTLDTNPYVKLLLDAMKHSGCAVNRGRHFSCEVCDGNVSGGFD
ASTSQIVLCENNIRNQAHMGRVVTHELIHAFDHCRAHVHWFTNIRHLACS
EIRAASLSGDCSLVNELFRLRFGLKQHHQTCVRDRAVLSILAVRNVSREE
AQKAVDEVFQTCFNDREPFGRIPHNQTYARYAHRDFQNRDRYYSNI
protein encoded by mouse Atp23 gene (Uniprot:
Q9CWQ3)
SEQ ID NO: 5
MAGAPGGGELGPAAGEPLLQRPDSGQGSPEPPAHGKPQQGFLSSLFTRDQ
SCPLMLQKTLDTNPYVKLLLDAMKHSGCAVNRGRHFSCEVCDGNVSGGFD
ASTSQIVLCENNIRNQAHMGRVVTHELIHAFDHCRAHVHWFTNIRHLACS
EIRAASLSGDCSLVNELFRLRFGLKQHHQIETSCVSRPAMNSQSCLGLVS
A

### REFERENCES

1. Hitti, F. L. & Siegelbaum, S. A. The hippocampal CA2 region is essential for social memory. Nature 508, 88-92 (2014).
2. Meira, T. et al. A hippocampal circuit linking dorsal CA2 to ventral CA1 critical for social memory dynamics. Nat. Commun. 9, 4163 (2018).
3. Chevaleyre, V. & Piskorowski, R. A. Hippocampal Area CA2: An Overlooked but Promising Therapeutic Target. Trends Mol Med. 22, 645-655 (2016).
4. Smith, A. S., Williams Avram, S. K., Cymerblit-Sabba, A., Song, J. & Young, W. S. Targeted activation of the hippocampal CA2 area strongly enhances social memory. Mol Psychiatry 21, 1137-1144 (2016).
5. Stevenson, E. L. & Caldwell, H. K. Lesions to the CA2 region of the hippocampus impair social memory in mice. Eur. J Neurosci 40, 3294-3301 (2014).
6. Dudek, S. M., Alexander, G. M. & Farris, S. Rediscovering area CA2: unique properties and functions. Nat. Rev. Neurosci. 17, 89-102 (2016).
7. Piskorowski, R. A. & Chevaleyre, V. Memory circuits: CA2. Curr. Opin. Neurobiol. 52, 54-59 (2018).
8. Piskorowski, R. A. et al. Age-dependent specific changes in area CA2 of the hippocampus and social memory deficit in a mouse model of the 22q11.2 deletion syndrome. Neuron 89, 163-176 (2016).
9. Scambler, P. J. et al. Velo-cardio-facial syndrome associated with chromosome 22 deletions encompassing the DiGeorge locus. Lancet 339, 1138-1139 (1992).
10. Karayiorgou, M., Simon, T. J. & Gogos, J. A. 22q11.2 microdeletions: linking DNA structural variation to brain dysfunction and schizophrenia. Nat Rev Neurosci 11, 402-416 (2010).
11. McDonald-McGinn, D. M. et al. 22q11.2 deletion syndrome. Nat Rev Dis Prim. 1, 15071 (2015).
12. Kragness, S., Harrison, M. A. A., Westmoreland, J. J., Burstain, A. & Earls, L. R. Age-dependent expression pattern in the mammalian brain of a novel, small peptide encoded in the 22q11.2 deletion syndrome region. Gene Expr. Patterns 28, 95-103 (2018).
13. Kann, O. The interneuron energy hypothesis: Implications for brain disease. Neurobiol. Dis. 90, 75-85 (2016).
14. Botcher, N. A., Falck, J. E., Thomson, A. M. & Mercer, A. Distribution of interneurons in the CA2 region of the rat hippocampus. Front. Neuroanat. 8, (2014).
15. DeVito, L. M. et al. Vasopressin 1b Receptor Knock-Out Impairs Memory for Temporal Order. J. Neurosci. 29, 2676-2683 (2009).
16. Brennan, P. A. & Zufall, F. Pheromonal communication in vertebrates. Nature 444, 308-315 (2006).
17. Stark, K. L. et al. Altered brain microRNA biogenesis contributes to phenotypic deficits in a 22q11-deletion mouse model. Nat Genet 40, 751-760 (2008).
18. Zhao, M., Choi, Y.-S., Obrietan, K. & Dudek, S. M. Synaptic Plasticity (and the Lack Thereof) in Hippocampal CA2 Neurons. J. Neurosci. 27, 12025-12032 (2007).
19. Nasrallah, K., Piskorowski, R. A. & Chevaleyre, V. Inhibitory Plasticity Permits the Recruitment of CA2 Pyramidal Neurons by CA3(1,2,3). eNeuro. 2, (2015).
20. Osman, C., Wilmes, C., Tatsuta, T. & Langer, T. Prohibitins Interact Genetically with Atp23, a Novel Processing Peptidase and Chaperone for the F1FO-ATP Synthase. Mol. Biol. Cell 18, 627-635 (2007).
21. Weckbecker, D., Longen, S., Riemer, J. & Herrmann, J. M. Atp23 biogenesis reveals a chaperone-like folding activity of Mia40 in the IMS of mitochondria. EMBO J. 31, 4348-4358 (2012).
22. Zeng, X., Neupert, W. & Tzagoloff, A. The metalloprotease encoded by ATP23 has a dual function in processing and assembly of subunit 6 of mitochondrial ATPase. Mol Biol Cell 18, 617-626 (2007).
23. Modjtahedi, N., Tokatlidis, K., Dessen, P. & Kroemer, G. Mitochondrial Proteins Containing Coiled-Coil-Helix-Coiled-Coil-Helix (CHCH) Domains in Health and Disease. Trends Biochem. 41, 245-260 (2016).
24. Rajendran, M., Dane, E., Conley, J. & Tantama, M. Imaging Adenosine Triphosphate (ATP). Biol. Bull. 231, 73-84 (2016).
25. Tantama, M., Martinez-Frangois, J. R., Mongeon, R. & Yellen, G. Imaging energy status in live cells with a fluorescent biosensor of the intracellular ATP-to-ADP ratio. Nat Commun 4, (2013).
26. Dimidschstein, J. et al. A viral strategy for targeting and manipulating interneurons across vertebrate species. Nat Neurosci 19, 1743-1749 (2016).
27. Nathanson, J. L. et al. Short Promoters in Viral Vectors Drive Selective Expression in Mammalian Inhibitory Neurons, but do not Restrict Activity to Specific Inhibitory Cell-Types. Front Neural Circuits. 3, 19 (2009).
28. Okuyama, T., Kitamura, T., Roy, D. S., Itohara, S. & Tonegawa, S. Ventral CA1 neurons store social memory. Science (80-.). 353, 1536-1541 (2016).
29. Cui, Z., Gerfen, C. R. & Young, W. S. Hypothalamic and other connections with dorsal CA2 area of the mouse hippocampus. J. Comp. Neurol. 521, 1844-1866 (2013).
30. Rowland, D. C. et al. Transgenically Targeted Rabies Virus Demonstrates a Major Monosynaptic Projection from Hippocampal Area CA2 to Medial Entorhinal Layer II Neurons. J. Neurosci. 33, 14889-14898 (2013).
31. Devaraju, P. & Zakharenko, S. S. Mitochondria in complex psychiatric disorders: Lessons from mouse models of 22q11.2 deletion syndrome. Bioessays 39, (2017).
32. Harris, J. J., Jolivet, R. & Attwell, D. Synaptic Energy Use and Supply. Neuron 75, 762-777 (2012).
33. Chevallier, C., Kohls, G., Troiani, V., Brodkin, E. S. & Schultz, R. T. The social motivation theory of autism. Trends Cogn. Sci. 16, 231-239 (2012).
34. Green, M. F., Horan, W. P. & Lee, J. Social cognition in schizophrenia. Nat Rev Neurosci 16, 620-631 (2015).
35. Piskorowski, R. A. & Chevaleyre, V. Memory circuits: CA2. Curr. Opin. Neurobiol. 52, 54-59 (2018).
36. Carstens, K. E. & Dudek, S. M. Regulation of synaptic plasticity in hippocampal area CA2. Curr. Opin. Neurobiol. 54, 194-199 (2019).
37. Moy, S. S. et al. Sociability and preference for social novelty in five inbred strains: an approach to assess autistic-like behavior in mice. Genes, Brain Behav. 3, 287-302 (2004).
38. Yang, M. & Crawley, J. N. Simple behavioral assessment of mouse olfaction.

Curr. Protoc. Neurosci. Chapter 8, Unit 8.24 (2009).
39. Sims, N. R. & Anderson, M. F. Isolation of mitochondria from rat brain using Percoll density gradient centrifugation. Nat. Protoc. 3, 1228-1239 (2008).
40. Kristian, T. Isolation of Mitochondria from the CNS. in Current Protocols in Neuroscience (John Wiley & Sons, Inc., 2001). doi:10.1002/0471142301.ns0722s52
41. Xu, P., Duong, D. M. & Peng, J. Systematical Optimization of Reverse-Phase Chromatography for Shotgun Proteomics. J. Proteome Res. 8, 3944-3950 (2009).
42. Pagala, V. R. et al. Quantitative Protein Analysis by Mass Spectrometry. in Protein-Protein Interactions: Methods and Applications (eds. Meyerkord, C. L. & Fu, H.) 281-305 (Springer New York, 2015). doi:10.1007/978-1-4939-2425-7_17
43. Wang, X. et al. JUMP: A Tag-based Database Search Tool for Peptide Identification with High Sensitivity and Accuracy. Mol. Cell. Proteomics 13, 3663-3673 (2014).
44. Denk, W. & Horstmann, H. Serial block-face scanning electron microscopy to reconstruct three-dimensional tissue nanostructure. PLoS. Biol. 2, e329 (2004).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method for treating a social memory deficit in a subject with a neuropsychiatric disease, said method comprising administering to the subject a therapeutically effective amount of (i) a protein encoded by a 2510002D24Rik gene or a functional derivative or fragment thereof, (ii) a vector expressing a protein encoded by a 2510002D24Rik gene or a functional derivative or fragment thereof,
(iii) a protein encoded by an Atp23 gene or a functional derivative or fragment thereof, or
(iv) a vector expressing a protein encoded by a Atp23 gene or a functional derivative or fragment thereof.

2-6. (canceled)

7. The method of claim 1, wherein the neuropsychiatric disease is selected from schizophrenia spectrum disorders, 22q11 deletion syndrome, and autism spectrum disorders.

8. The method of claim 1, wherein the vector is selected from adeno-associated virus (AAV) vectors, retrovirus vectors, adenovirus vectors, Sindbis virus vectors, vaccinia virus vectors, and herpes virus vectors.

9. The method of claim 8, wherein the vector is an AAV vector.

10-11. (canceled)

12. The method of claim 1, wherein the expression of the protein or functional derivative or fragment thereof is controlled by a promoter selected from a pan-GABAergic interneuron promoter, fsst promoter, hDlx promoter, mDlx promoter, Synapsin promoter, CMV promoter, β-actin promoter, and CamKIIa promoter.

13. (canceled)

14. The method of claim 1, wherein the administration is via injection into the CA2 area of the hippocampus of the subject.

15. The method of claim 1, wherein the administration is via a transcranial surgical injection.

16. The method of claim 1, wherein the administration is systemic.

17. The method of claim 1, wherein the administration is intranasal.

18. The method of claim 1, wherein the protein encoded by said 2510002D24Rik gene comprises the amino acid sequence which has at least 80% sequence identity to SEQ ID NO: 1.

19. The method of claim 18, wherein the protein encoded by said 2510002D24Rik gene comprises the amino acid sequence which has at least 90% sequence identity to SEQ ID NO: 1.

20. The method of claim 19, wherein the protein encoded by said 2510002D24Rik gene comprises the amino acid sequence SEQ ID NO: 1.

21. The method of claim 20, wherein the protein encoded by said 2510002D24Rik gene consists of the amino acid sequence SEQ ID NO: 1.

22. The method of claim 1, wherein the protein encoded by said Atp23 gene comprises the amino acid sequence which has at least 80% sequence identity to SEQ ID NO: 2.

23. The method of claim 22, wherein the protein encoded by said Atp23 gene comprises the amino acid sequence which has at least 90% sequence identity to SEQ ID NO: 2.

24. The method of claim 23, wherein the protein encoded by said Atp23 gene comprises the amino acid sequence SEQ ID NO: 2.

25. The method of claim 24, wherein the protein encoded by said Atp23 gene consists of the amino acid sequence SEQ ID NO: 2.

26. The method of claim 1, wherein the subject is human.

27. A pharmaceutical composition comprising (i) a protein encoded by a 2510002D24Rik gene or a functional derivative or fragment thereof and a pharmaceutically acceptable carrier or excipient, or (ii) a vector encoding a protein encoded by a 2510002D24Rik gene or a functional derivative or fragment thereof and a pharmaceutically acceptable carrier or excipient.

28-37. (canceled)

38. A pharmaceutical composition comprising (i) a protein encoded by said Atp23 gene or a functional derivative or fragment thereof and a pharmaceutically acceptable carrier or excipient, or (ii) a vector encoding a protein encoded by said Atp23 gene or a functional derivative or fragment thereof and a pharmaceutically acceptable carrier or excipient.

39-50. (canceled)


SEQUENCE LISTING (41 sequences)

SEQ ID NO: 1 - protein, 142 aa, Homo sapiens
MCRCSLVLLSVDHEVPFSSFFIGWRTEGRAWRAGRPDMADGSGWQPPRPCEAYRAEWKLCRSARHFLHHYYVHGERPACEQWQRDLASCRDWEERRNAEAQQSLCESERARVRAARKHILVWAPRQSPPPDWHLPLPQEKDE

SEQ ID NO: 2 - protein, 246 aa, Homo sapiens
MAGAPDERRRGPAAGEQLQQQHVSCQVFPERLAQGNPQQGFFSSFFTSNQKCQLRLLKTLETNPYVKLLLDAMKHSGCAVNKDRHFSCEDCNGNVSGGFDASTSQIVLCQNNIHNQAHMNRVVTHELIHAFDHCRAHVDWFTNIRHLACSEVRAANLSGDCSLVNEIFRLHFGLKQHHQTCVRDRATLSILAVRNISKEVAKKAVDEVFESCFNDHEPFGRIPHNKTYARYAHRDFENRDRYYSNI

SEQ ID NO: 3 - protein, 105 aa, Mus musculus
MAVAGSWQPPRPCEVYRAEWELCRSVGHVLHHYYVHGKRPDCRQWLRDLTNCREWEESRSAEAQRSLCESEQVRVQAAQKHTLVWALRQRPPTDWNLPLPQEKDK

SEQ ID NO: 4 - protein, 246 aa, Mus musculus
MAGAPGGGELGPAAGEPLLQRPDSGQGSPEPPAHGKPQQGFLSSLFTRDQSCPLMLQKTLDTNPYVKLLLDAMKHSGCAVNRGRHFSCEVCDGNVSGGFDASTSQIVLCENNIRNQAHMGRVVTHELIHAFDHCRAHVHWFTNIRHLACSEIRAASLSGDCSLVNELFRLRFGLKQHHQTCVRDRAVLSILAVRNVSREEAQKAVDEVFQTCFNDREPFGRIPHNQTYARYAHRDFQNRDRYYSNI

SEQ ID NO: 5 - protein, 201 aa, Mus musculus
MAGAPGGGELGPAAGEPLLQRPDSGQGSPEPPAHGKPQQGFLSSLFTRDQSCPLMLQKTLDTNPYVKLLLDAMKHSGCAVNRGRHFSCEVCDGNVSGGFDASTSQIVLCENNIRNQAHMGRVVTHELIHAFDHCRAHVHWFTNIRHLACSEIRAASLSGDCSLVNELFRLRFGLKQHHQIETSCVSRPAMNSQSCLGLVSA

SEQ ID NO: 6 - DNA, 25 bp, Artificial Sequence
  Description of Artificial Sequence Synthetic primer
accatgtgaatctactgcctgaggg

SEQ ID NO: 7 - DNA, 25 bp, Artificial Sequence
  Description of Artificial Sequence Synthetic primer
tatgtgggtgaatgcctgtagtccc

SEQ ID NO: 8 - DNA, 22 bp, Artificial Sequence
  Description of Artificial Sequence Synthetic primer
gctcacctacactctgtgtatg

SEQ ID NO: 9 - DNA, 22 bp, Artificial Sequence
  Description of Artificial Sequence Synthetic primer
gtgtagatgggcgcatcgtaac

SEQ ID NO: 10 - DNA, 24 bp, Artificial Sequence
  Description of Artificial Sequence Synthetic oligonucleotide
agggcctccctctaccacaggaga

SEQ ID NO: 11 - DNA, 24 bp, Artificial Sequence
  Description of Artificial Sequence Synthetic oligonucleotide
aaactctcctgtggtagagggagg

SEQ ID NO: 12 - DNA, 200 bp, Artificial Sequence
  Description of Artificial Sequence Synthetic polynucleotide
tccaggctgctcagaagcacaccttggtatgggccttgaggcaaaggccccctacggactggaacctccctctaccacaggagaaagacaagtacccatacgatgttccagattacgcttgataagcaactgcaaccctacccttgacctagactttgactggctcttacttgacctggaacacagagcaaagcatttcc

SEQ ID NO: 13 - DNA, 23 bp, Artificial Sequence
  Description of Artificial Sequence Synthetic primer
ctctgtgagagcgagcaggttcg

SEQ ID NO: 14 - DNA, 20 bp, Artificial Sequence
  Description of Artificial Sequence Synthetic primer
tccagtgggcagcaggcaat

SEQ ID NO: 15 - DNA, 24 bp, Artificial Sequence
  Description of Artificial Sequence Synthetic oligonucleotide
agggccagtaagagagaagaacgg

SEQ ID NO: 16 - DNA, 24 bp, Artificial Sequence
  Description of Artificial Sequence Synthetic oligonucleotide
aaacccgttcttctctcttactgg

SEQ ID NO: 17 - DNA, 24 bp, Artificial Sequence
  Description of Artificial Sequence Synthetic oligonucleotide
agggtcatagcggatgtgggacag

SEQ ID NO: 18 - DNA, 24 bp, Artificial Sequence
  Description of Artificial Sequence Synthetic oligonucleotide
aaacctgtcccacatccgctatga

SEQ ID NO: 19 - DNA, 200 bp, Artificial Sequence
  Description of Artificial Sequence Synthetic polynucleotide
ccatctttttagctcccttcctccctccctcctttcctctccctcctcagaggtgagaactgaacctaagaccttgtgcttaccaggcaaggccctaccatggctggtgacgctgttctgcggtttcatgcacgttctcccggctgctgctttgctgatgttctcttctgccctcaagcgttgaagtcccacagaccttt

SEQ ID NO: 20 - DNA, 23 bp, Artificial Sequence
  Description of Artificial Sequence Synthetic primer
tcctccctccctcctttcctctc

SEQ ID NO: 21 - DNA, 25 bp, Artificial Sequence
  Description of Artificial Sequence Synthetic primer
cctcggcagaacacagaagaagaaa

SEQ ID NO: 22 - DNA, 27 bp, Artificial Sequence
  Description of Artificial Sequence Synthetic primer
aatgagggttagcaagcagaaatgtgt

SEQ ID NO: 23 - DNA, 25 bp, Artificial Sequence
  Description of Artificial Sequence Synthetic primer
gagtgcaccatatggacatattgtc

SEQ ID NO: 24 - DNA, 25 bp, Artificial Sequence
  Description of Artificial Sequence Synthetic primer
taatgcaggttaacctggcttatcg

SEQ ID NO: 25 - DNA, 20 bp, Artificial Sequence
  Description of Artificial Sequence Synthetic primer
cttggtcaggctggaaagag

SEQ ID NO: 26 - DNA, 23 bp, Artificial Sequence
  Description of Artificial Sequence Synthetic primer
agtatggctttccttcccgatgg

SEQ ID NO: 27 - DNA, 24 bp, Artificial Sequence
  Description of Artificial Sequence Synthetic primer
tctaaggccgagtcttatgagcag

SEQ ID NO: 28 - DNA, 20 bp, Artificial Sequence
  Description of Artificial Sequence Synthetic primer
tcctagccaaatggatggac

SEQ ID NO: 29 - DNA, 25 bp, Artificial Sequence
  Description of Artificial Sequence Synthetic primer
tcgttcgaacataacttcgtatagc

SEQ ID NO: 30 - DNA, 28 bp, Artificial Sequence
  Description of Artificial Sequence Synthetic primer
ccagatgatctaagtatatgtgttgcac

SEQ ID NO: 31 - DNA, 27 bp, Artificial Sequence
  Description of Artificial Sequence Synthetic primer
cctaactggagatcataaggtgagatg

SEQ ID NO: 32 - DNA, 23 bp, Artificial Sequence
  Description of Artificial Sequence Synthetic probe
cttctttcacccttagtcatcct

SEQ ID NO: 33 - DNA, 22 bp, Artificial Sequence
  Description of Artificial Sequence Synthetic probe
gcagtttggtactcaggagaga

SEQ ID NO: 34 - DNA, 18 bp, Artificial Sequence
  Description of Artificial Sequence Synthetic primer
gtgttccaggtcaagtaa

SEQ ID NO: 35 - DNA, 19 bp, Artificial Sequence
  Description of Artificial Sequence Synthetic primer
agaaggacaagtgataagc

SEQ ID NO: 36 - DNA, 20 bp, Artificial Sequence
  Description of Artificial Sequence Synthetic primer
cgcttcggcagcacatatac

SEQ ID NO: 37 - DNA, 20 bp, Artificial Sequence
  Description of Artificial Sequence Synthetic primer
ttcacgaatttgcgtgtcat

SEQ ID NO: 38 - DNA, 24 bp, Artificial Sequence
  Description of Artificial Sequence Synthetic primer
taaagcttatggcaggagctccgg

SEQ ID NO: 39 - DNA, 27 bp, Artificial Sequence
  Description of Artificial Sequence Synthetic primer
taaagctttcatatgttggagtagtag

SEQ ID NO: 40 - DNA, 30 bp, Artificial Sequence
  Description of Artificial Sequence Synthetic primer
atgtcgaccaccatgtccaatttactgacc

SEQ ID NO: 41 - DNA, 28 bp, Artificial Sequence
  Description of Artificial Sequence Synthetic primer
atggcgcgccttacttgtaaagctcgtc